SEQUENCE LISTING (97 sequences)

SEQ ID NO: 1 - DNA, 796 bp, Nicotiana tabacum
  misc_feature 793..796: note = n = A,T,C or G
  source 1..796: mol_type = unassigned DNA
atggaacatcaaggattaagcaccaatatgaggagaatcctcctgctgattaactgtctaatactcgctgttggtatctgtggtggtcctctaatgatgcgtctatattatagtagggtttggttacaaactgctggatggccactcacccttatacctcttgccatcctatacttttatcgtcgaaaaaccgaaggctctaatgccaagttttactttataacaccccgaattttcattgcatcattcgtcattggcgttgtcactggtcttgatgattttctctattcgtggggcgggtcaaaactccctgtgtcaacctcttcacttcttcttgctgctcaacttgccttcacggcaataggtgctttcttcatagtgaagctgaagttcacaccctactctatcaatgcagtggttctgttgacagttggtgctgttttattgggtattcgatctaatggtgatcgaccagagggtgtgacaagtaaagcctatattcttggttttatgatgacacttctggcagcagctttatatggagtcattttgccttgtattgagttgatttacttgaaggcaaaacaagctattactgctacgttggtattggagattcaaatggtcatgtattttgccttgtattgagttgatttacttgaaggcaaaacaagctattactgctacgttggtgttggagattcagatggtcatgtgttttgctgctactgctttttgcaccgtaggaatgatcgccaataacgattttcaggtacttttttcttcccttgtnnnn

SEQ ID NO: 2 - protein, 215 aa, Nicotiana tabacum
MEHQGLSTNMRRILLLINCLILAVGICGGPLMMRLYYSRVWLQTAGWPLTLIPLAILYFYRRKTEGSNAKFYFITPRIFIASFVIGVVTGLDDFLYSWGGSKLPVSTSSLLLAAQLAFTAIGAFFIVKLKFTPYSINAVVLLTVGAVLLGIRSNGDRPEGVTSKAYILGFMMTLLAAALYGVILPCIELIYLKAKQAITATLVLEIQMVMYFALY

SEQ ID NO: 3 - DNA, 975 bp, Nicotiana tabacum
  source 1..975: mol_type = unassigned DNA
atggaacatcaaggattaagcaccactatgaggagaatcctcctgctcattaactgtttaatactcgctgttggtatctgcggtggtcctctaatgatgcgtctatattatgtcgagggaggttcaagagtatggtttagcagttggttacaaactgctggatggccattcacccttatacctcttgccatcctatacttctatcgtcgaaaaaccgaaggcgctaatgccaagtattacttgataacaccccgaattttcatcgcatcattcatcattggcgttgtcactggtcttgatgattttctctattcgtggggcgggtcaaaacttcctgtgtcaacctcttcacttcttcttgctgctcaacttgccttcacggcaataggtgctttcttcatagtgaagctgaagttcacaccctactctatcaatgcagtggttctgttgacagttggtgctgttttattaggtgttcgatctaatggtgatcggccagagggtgtgacaagtaaagcctatattcttggttttatgatgacacttttggcagcagctttatatggagtcattttgccttgtattgagttgatttacttgaaggcaaagcaagctattactgctacattagtattggagattcaaatggtcatgtgttttgctgctactgctttttgcactgttggaatgatcgccaataacgactttcagtgcttctttgtgggtgtcattggagtcatttactgctcttcttctttgatgtctggggttatgatcgcagttctacttcctgttactgaggtattagctgtagttttctttagggaaaatttttcaggtgaaaagggccttgctctttttctttctctttggggtttcgtctcatacttttacggagagttcagacaaacaaagaagcagaagaacaaaagtccaaaaactgagatgacaacaacgcataccgagtctgtttaa

SEQ ID NO: 4 - protein, 324 aa, Nicotiana tabacum
MEHQGLSTTMRRILLLINCLILAVGICGGPLMMRLYYVEGGSRVWFSSWLQTAGWPFTLIPLAILYFYRRKTEGANAKYYLITPRIFIASFIIGVVTGLDDFLYSWGGSKLPVSTSSLLLAAQLAFTAIGAFFIVKLKFTPYSINAVVLLTVGAVLLGVRSNGDRPEGVTSKAYILGFMMTLLAAALYGVILPCIELIYLKAKQAITATLVLEIQMVMCFAATAFCTVGMIANNDFQCFFVGVIGVIYCSSSLMSGVMIAVLLPVTEVLAVVFFRENFSGEKGLALFLSLWGFVSYFYGEFRQTKKQKNKSPKTEMTTTHTESV

SEQ ID NO: 5 - DNA, 1160 bp, Nicotiana tabacum
  source 1..1160: mol_type = unassigned DNA
atggaagataaaggattaagcaccaatatgaggagaatcctcctactgattaactgtctaatactcgctgttggtatttgtggtggccctctaatgatgcgtctatattatgtcgaaggaggtatggttgcatgaggagaatactcctgttggttaactgtctaatactcgctgttggtgtttgtggtggtcctcttatgatgcgcctatattatgtcgaaggaggttcaagagtatggttcagtagttggttacaaactgctggatggccactcacccttatacctcttgccatcctatacttctatcgtcgaaaaaccgaaggctctaatgccaagttttactttataacaccccgaattttcattgcatcattcatcattggcgttgtcactggtgttgatgattttctctattcgtggggcgggtcaaaactccctgtgtcaacctcttcacttcttcttgctgctcaacttgccttcacggcagtaggtgctttcttcatagtaaagttaaagttcacaccctactccatcaatgcagtgattttgttgacagttggtgctgttttattgggtgttcgatctaatggtgatcgaccagaaggtgtgacaagtaaagcctatattcttggttttatgatgacacttctggcagcagctttgtatggagtcattttgccttgtattgagttgatttacttgaaggcaaaacaagctattactgctacgttggtgttggagattcagatggtcatgtgttttgctgctactgctttttgcaccgtaggaatgatagcaatatcaagggaggcaaaacaatttaacctcggagaagctagatattatacagtgatagtatggactgccattatttggcaatgcttctttgtgggtgttattggagtcatttactgctcttcttctttgatgtctggtgttatgatcgcagttttactccctgttactgaggtattagctgtagttttttttagggaaaagttttcaggtgaaaagggccttgctttattcctttctctttggggtttcgtctcatacttttacggagagttcagacaaacaaagaagcagaagaataaaagtccagaaactgagatgacaatgatgcataccgagtctgtttgatttattgagactgtt

SEQ ID NO: 6 - protein, 337 aa, Nicotiana tabacum
MRRILLLVNCLILAVGVCGGPLMMRLYYVEGGSRVWFSSWLQTAGWPLTLIPLAILYFYRRKTEGSNAKFYFITPRIFIASFIIGVVTGVDDFLYSWGGSKLPVSTSSLLLAAQLAFTAVGAFFIVKLKFTPYSINAVILLTVGAVLLGVRSNGDRPEGVTSKAYILGFMMTLLAAALYGVILPCIELIYLKAKQAITATLVLEIQMVMCFAATAFCTVGMIAISREAKQFNLGEARYYTVIVWTAIIWQCFFVGVIGVIYCSSSLMSGVMIAVLLPVTEVLAVVFFREKFSGEKGLALFLSLWGFVSYFYGEFRQTKKQKNKSPETEMTMMHTESV

SEQ ID NO: 7 - DNA, 1223 bp, Nicotiana tabacum
  source 1..1223: mol_type = unassigned DNA
ccttcggagaagaaaagcatttcagcctgagtaacagaatactccaatttagtatccaaatctctagaaaaaaaatatggaacatccaggattaagcaccaatatgaggagaatcctcctgctgattagctgcttaatactcgctgttggcatatgtggtggtcctctaatgatgcgtctatattatgtcgagggaggttcaagaatatggcttagcagttggttacaaactggtggatggccactcacccttatacctcttgccatcctatactattatcgtcgaaaaaccgaaggctcaaacgccaagttttacttgatgacaccccgaatttttattgcatcatttgttattggcgttgccactggtcttgatgattttctctattcgtggggcgggtcaaaactccccgtatcaacctcttcacttcttcttgctgctcaacttgccttcacggcagtaggtgggtcaaaactccccgtatcaacctcttcacttcttcttgctgctcaacttgccttcacggcagtaggtgctttcttcatagtgaagttgaagttatcacccttctctatcaatgcagtggttttactaacagttggtgctgttttattgggtattcgatctaatggtgatcgaccagagggcgtgacaagtaaagaatatattattggttttatgatgacacttctagcagcagctttgtacggagtcattttgccttgtattgagttgatttacatgaaggcaaaacaagctattacttctacgttagtattggagattcagatgatcatgtgttttgctgctactgctttttgcactgtaggaatgatcgccaataaggactttcaggcaatgtcaagggaggcaaaacaatttaacctcggagaagctagatattatacagttatagtatgcactgccgctatttggcagtgcttcttcgtgggtattattggagttatctactgctcttcttctttgatgtctggggttatgattgcagttttgctccccgttactgaggtattagctgtaattttctttaaggaaaatttttcaggtgaaaagggtcttgctcttttcctttctctttggggtttcgtctcatacttctatggagagttcagacaaacaaagaagcagaagaataaaagtccagaagctgagatgacaaggacaacgacgcatactgaatctgtttgatttactattcatg

SEQ ID NO: 8 - protein, 377 aa, Nicotiana tabacum
MEHPGLSTNMRRILLLISCLILAVGICGGPLMMRLYYVEGGSRIWLSSWLQTGGWPLTLIPLAILYYYRRKTEGSNAKFYLMTPRIFIASFVIGVATGLDDFLYSWGGSKLPVSTSSLLLAAQLAFTAVGGSKLPVSTSSLLLAAQLAFTAVGAFFIVKLKLSPFSINAVVLLTVGAVLLGIRSNGDRPEGVTSKEYIIGFMMTLLAAALYGVILPCIELIYMKAKQAITSTLVLEIQMIMCFAATAFCTVGMIANKDFQAMSREAKQFNLGEARYYTVIVCTAAIWQCFFVGIIGVIYCSSSLMSGVMIAVLLPVTEVLAVIFFKENFSGEKGLALFLSLWGFVSYFYGEFRQTKKQKNKSPEAEMTRTTTHTESV

SEQ ID NO: 9 - DNA, 4761 bp, Nicotiana tabacum
  source 1..4761: mol_type = unassigned DNA
atgtgctgcttttgcgggaaaatcttccctccattcattttcctctctccatgtccacaaaggactcttctatcttccattgatgtgctgcttttgcttactttcattgtatttgcagtacaaaagttgtactcaaagttgaggtccaatgagcactctacttctagcattgataagcctctaattgcacacaacaggacttctgttagaaccaatctttggtttaagctgtctctgattttgtcagctattttagccttatcttctatagttttatgcattttggttattgtgggaaattcccagtcgccttggaaagtcatagatggactgtattggttgtttcaggcgattacacatgttgtaatcactatactaatagttcatgagaaaagatttcacgctatttcccatccactgtccctgcgcgtgttttggattgcaaactttgtagttatgagtttgttctttggttgtggagtcacaaggcttgtgtcacttaaggaaattgatcctaatttaagaatggatgatataagttcattagtttcatttcctatttctgttgttctcttcattgttgccattaaaggttcgaccggagttgctgtaattagtgattctgaatctcacttaagtgatgaaaccaatggttatgaactcctggataaatccagtgtgagtggctttgcttcagcttctctaatatcgaaagccttttggatttggatgaaccctttactgcaaaaaggttacaagtcacctctcaagattgatgaagttccttcactttccccactgcatagagcagagaaaatgtctcaacttttcgaaagaaattggcctaaacctgaagaaatatcaaagcatcctgtccgaacaacattgctgcgttgcttttggaaggaagttatttttactgccattcttgcagtaattagggtatgtgttatgtatgtagggccaacactcatacaaagatttgttgattacacagcaggaaagaggacatctccttatgaaggatactaccttataggaactctcctaatagccaaatttgtggaagtcaggacatctccttatgaaggatactaccttataggaactctcctaatagccaaatttgtggaagttctaacctctcatcagttcaactttaactcccaaaagcttggcatgcttattcgagcgacacttctcacttctttgtataagaaggggttaaggttgtcatgctcagctagacaggctcatggtgttggacagattgtaaattatatggccgtcgatgctcagcagctgtccgatatgatgctacagctacattccatttggctcatgccattgcaagtttctgtggctttaggcatcctttatacttacctcggtgcttcaactgttgtaacgctagctggacttgcagcagtgatggtatttgtggtgtttggaactaaaagaaacaacaggtttcaatttaacatcatgaagaatcgtgattctagaatgaaagcgacaaatgagatgcttaattatatgcgcgttataaagttccaggcatgggaagaacattttaacaaaagaattgaatccttccgcgaatccgagtatggatggttgtccaagttcttgtactcaatcgctgggaatatcattgtcttgtggagcactcctcttctagtggctacactcacttttggaagtgcaatcttgttgggaatcccgcttggtgcagggacagtgttcactgcaacatctctcttcaagatgttgcaggaaccgatcagggctttccctcaatccatgatctcactttcacaagcaatgatatctcttgatagattggacaaatatatgatgagtaaggagttagtggataaagctgtggaaagactagaaggttgtgggggtacaattgctatgcaggtgaaagatggagctttttgctgggatgatgaaaacagtaaagaagaattgaaaaatgtaaactttgagattagaaaaggagagcttgcagcagtagtggggacagttggggcggggaagtcttccctccttgcatctgtacttggtgagatgcacaagttgtcgggtcaggtcacaatttgtggttcaactgcctatgttgcacaaacatcgtggattcagaatggcacgatacaagaaaatatcctgtttggtatgccaatgaacagagacagatacaaggaagtgatccgggtttgctgcttggagaaggacttggaaataatggagtttggagaccagactgaaataggagaacgtggcatcaacctcagtggtggtcagaagcagcgaatccagcttgcaagagctgtttaccaggactgtgatatttatcttctagatgatgtattcagtgcagttgatgctcacactggctctgaaatcttcaaggaatgtgtgaggggaattcttaaagataaaaccattttgcttgtcacacaccaagttgacttcttgcataatgttgacctgatccttgtcatgcgagatgggatgatcgtgcaatctggcaaatataatgagatattagaagctggaatggattttaaagagctagtagctgcacatgagacctctttagaacttgttgacgtggaaacaaccaaagagagcaatgcctcccttgaagaatcaaaatcttctcgaagattatctaaggaagaaaacggagatgataaatctcaacagtctacatctgataggggggattctaaacttataaaggaagaagaaagagaaactggaaaagtcagtcctcgtgtgtacaagctatatattactgaagcttttggatggtggggtgtagtgctagttatcttgttttcgttcttgtggcaaagttctctaatggcaagtgattattggctggcatatgaaacttcagcggatcgtgccatgtccttcaatccttctctgtttattgggatatacggtgttattgcagttgtttcttcgttgctgatagtgatcaggatgtattttgtgacacttatggggctcaagactgcccaaatatttttcggacagattctttacagcatactgcatgctcctatgtcattttttgacacaacaccttccggaagaattctgagtcgggcatctaatgatcagaccaacattgatgtcttcctcccgttttttatgaatctcactttggccatgtttatcacactgctcggcatcatcatcatcacatgccaatattcttggcctaccgtactacttttgattcctctgggttggcttaatatctggtaccggggatattatcttgcaacatctcgtgaattgactcggcttgactcaattacaaaagcacctgttattcatcatttctctgaaagcatctcaggtgttatgactatacgttgctttaggaagcaggagatgttttgtaacgagaatgtaaaccgagtgaattccaatctgcgaatggatttccacaacaatggatccaatgaatggttgggctttcgactggaattgatgggaagcttacttctttgtgtttctgcaatgttcatgattgtcttacctagcagcatcatcaagccagaaaatgttggtttgtcactatcatatggcttgtctcttaatagtgtcctattctggtccatctttgtgagttgctttgtggaaaataaaatggtttctgtcgaaagattaaaacagttctcagaaataccatcagaagcagagtggagaaagatggattttctcccaccttcaagttggccaagccgtgggaatgttgagcttgaaaacgtgcaggttagatatcgtccgaacactcctctagtgcttaaaggagttactctcagcattagagggggagagaagataggtgttgttggtcgtacagggggtggaaaatcaacattaattcaagttttctttcgtttggtggagcctgcagctggaagaataatcattgatgacgtagatatatccagacttgggcttcatgatcttagatctcgcttcgggatcattccccaagagccagtcctttttgaaggaactgtgagaagcaacattgaccccattggacaatattcagatgatgaaatttggaagagcctcgaacgctgccaactcaaagatgtggtgtctttaaaacccgaaaaacttgattcaccaggtaccttttccttctctactgcatctcttctgagttatacaatcaactttttatgcttggcaatatatatactgaatactgtcattggtaaaatagttgttgataacggagataactggagtgtcggacagaggcagcttctttgcttgggaagagtgatgctaaaacgtagcagacttctatttatggatgaggcaactgcctctgttgattcacagacagatgcagtgattcagaaaatcatccgcgaggactttgcggcctgtactataatcagcattgcccacagaataccaacagtcatggactgtgatagagttcttgttatagatgcaggaatagcaaaagagtttgacaaaccatctcgtttgcttgaaaggccttcactttttggggctttggttcaagaatatgccaaccgatcctctgagctctaaccacactaatcaggctttcatgccttttgctgtaaattgctatcttggacgatagtaggtgaaacaggaaaaataatgttacatagatttccaaatagtgtcatctcctccttagtctgtccagtagattttcggaaatgtaacaacattggg

SEQ ID NO: 10 - protein, 1535 aa, Nicotiana tabacum
MCCFCGKIFPPFIFLSPCPQRTLLSSIDVLLLLTFIVFAVQKLYSKLRSNEHSTSSIDKPLIAHNRTSVRTNLWFKLSLILSAILALSSIVLCILVIVGNSQSPWKVIDGLYWLFQAITHVVITILIVHEKRFHAISHPLSLRVFWIANFVVMSLFFGCGVTRLVSLKEIDPNLRMDDISSLVSFPISVVLFIVAIKGSTGVAVISDSESHLSDETNGYELLDKSSVSGFASASLISKAFWIWMNPLLQKGYKSPLKIDEVPSLSPLHRAEKMSQLFERNWPKPEEISKHPVRTTLLRCFWKEVIFTAILAVIRVCVMYVGPTLIQRFVDYTAGKRTSPYEGYYLIGTLLIAKFVEVRTSPYEGYYLIGTLLIAKFVEVLTSHQFNFNSQKLGMLIRATLLTSLYKKGLRLSCSARQAHGVGQIVNYMAVDAQQLSDMMLQLHSIWLMPLQVSVALGILYTYLGASTVVTLAGLAAVMVFVVFGTKRNNRFQFNIMKNRDSRMKATNEMLNYMRVIKFQAWEEHFNKRIESFRESEYGWLSKFLYSIAGNIIVLWSTPLLVATLTFGSAILLGIPLGAGTVFTATSLFKMLQEPIRAFPQSMISLSQAMISLDRLDKYMMSKELVDKAVERLEGCGGTIAMQVKDGAFCWDDENSKEELKNVNFEIRKGELAAVVGTVGAGKSSLLASVLGEMHKLSGQVTICGSTAYVAQTSWIQNGTIQENILFGMPMNRDRYKEVIRVCCLEKDLEIMEFGDQTEIGERGINLSGGQKQRIQLARAVYQDCDIYLLDDVFSAVDAHTGSEIFKECVRGILKDKTILLVTHQVDFLHNVDLILVMRDGMIVQSGKYNEILEAGMDFKELVAAHETSLELVDVETTKESNASLEESKSSRRLSKEENGDDKSQQSTSDRGDSKLIKEEERETGKVSPRVYKLYITEAFGWWGVVLVILFSFLWQSSLMASDYWLAYETSADRAMSFNPSLFIGIYGVIAVVSSLLIVIRMYFVTLMGLKTAQIFFGQILYSILHAPMSFFDTTPSGRILSRASNDQTNIDVFLPFFMNLTLAMFITLLGIIIITCQYSWPTVLLLIPLGWLNIWYRGYYLATSRELTRLDSITKAPVIHHFSESISGVMTIRCFRKQEMFCNENVNRVNSNLRMDFHNNGSNEWLGFRLELMGSLLLCVSAMFMIVLPSSIIKPENVGLSLSYGLSLNSVLFWSIFVSCFVENKMVSVERLKQFSEIPSEAEWRKMDFLPPSSWPSRGNVELENVQVRYRPNTPLVLKGVTLSIRGGEKIGVVGRTGGGKSTLIQVFFRLVEPAAGRIIIDDVDISRLGLHDLRSRFGIIPQEPVLFEGTVRSNIDPIGQYSDDEIWKSLERCQLKDVVSLKPEKLDSPGTFSFSTASLLSYTINFLCLAIYILNTVIGKIVVDNGDNWSVGQRQLLCLGRVMLKRSRLLFMDEATASVDSQTDAVIQKIIREDFAACTIISIAHRIPTVMDCDRVLVIDAGIAKEFDKPSRLLERPSLFGALVQEYANRSSEL

SEQ ID NO: 11 - DNA, 3684 bp, Nicotiana tabacum
  source 1..3684: mol_type = unassigned DNA
atgagtactactacgaagagtaaagcgatgatacaaaagaaaagctacggctcttttgtgtcaattttcatgcatgccgatagagcagatatcttgttgatgattttaggtttttttggagcagtttgcgacgggttttccatgccgataatgctcatagtcaccagcaaactcatgaacaatcttggtggtactgacacttcaaacgccgataatttcacgcaccacatcaatgagaatgctttggttctggttttccttgcatgtggacaatgggtcgcttgtttcttagagggattttgttggacaagaacagcagagaggcaagcttcaaggctacgaataagatacttgaaagcagttctaaggcaagatgttggctactttgatctacatgttgctagcacagccgacgttataactagcgtctcaagtgacagtcttgtcattcaagactgcattagtgaaaaggttccagttttcttgatgaacgtagcagcttttattggatcatacgtggtaggattcttgatgctatggaggctggcattagtaggatttccctttgttattttcttagtaataccaggtctcatgtacgggcgggctctaatgggcatagtcagaaaaatcagggacgaatatggcaaagctggaacagttgtggagcaagcaatttcttcggttagaacagtttattcgtttgtaggagaaaacaaaactatagcagagtactctgttgcactacaaggaactgtagaattgggactaaagcaaggtttagcaaaaggtttggctattggaagtaatggcatagtatttgcaatttggtccttcatgtcttattatggtagcagattggtcatgtataatggagcacatggtggcaccgtctttgccgtcggcgctgcaattgctatcggtggactatcattgggttctggtttgtccaacgtgaagtatttttccgaagcaagtgcagctggtgaacgagtaatggaagtgataaaaagggtaccaaaaatagactcagataacatggaaggaaaaacagtagcattagtgggcggaagtggatctgggaaatcaacagtagtagcattgctacagagattttacgaaccacttgcaggagagatatttgtggatggggtcgctattgaaaagttgcagctcaagtggctaaggtctcaaatgggtttagtgagtcaagagccagcactttttgcaactacaattaaggaaaatatactttttgggaaagaggatgcatctatggaacaagttattgaggctgcaaaagcttctaatgctcataacttcatcagtcagttgcctcagggttatgatacccaggtgggggagagaggtgtacaaatgtcaggggggcagaagcagaggatagccatagcaagagccacaatcaaatcgcccagaatactcctcctagacgaggcaaccagtgcacttgactctgaatcagagcgcgtcgtgcaggaagctctagacatggccgctgttggccgcaccacaatcatcatagcccatcgcctctccactattcgcaatgctgaccttatagccgtggtccaagatggtcaagtcaaggaaattggctcacacgacgacctgattgaagaggaaaatggactatacacctccttagtccgtctccaacaaactgaaaaaccaagtgatgaattctacattgcacctaccaataaaaacattgtctttgccccatcaaatttaaatcctggatccgcctctgactatgacatacaaaatacaagtagccgaaggctttcaattgtgagccggtcaagttcagcaaactcagctgcgcaaagccgtagagtggatcaaaatgcaacaatttccagtactacagaacaattatttcctgtaccttcatttaaaaggctactggctatgaatttgccagaatggaaggaagcaacgctgggatgtataggagcaatattatttggaggagttcaaccagtgtatgcttttgcaatggggtcaatgatttctgtctatttcttgcctagtcatgatgagattaaggagaagactaagatatatgctctgtgtttcctcgggctggcattcttttcccttattgtcaatgtacttcagcactataattttgcagccatgggagagcaattgactaaaagggttagggaaaggatgttgtccaagatgcttacttttgaaattgggtggtatgacaaggacgagaattccactggtgctgtttgctctagactagccaaggatgccaatgtggtgaggtctttggtcggggacaggatggcactactcattcaaacagtctcggcagtgaccattgcttgcaccatgggcctagttatcgcgtggaaacttgcattggttatgatcgcggtccagcctctcatcatagtgtgctactattgcaagagagtgctattgaaaagtatgtctaagaaatccataaagtcacaagaagaaagcagcaagttggcagctgaagcagtttccaatctcagaactgtaacagccttctcctcccaggcacgaatcctacaaatgctcaagaaagcccaagaaggcccacaacgagaaagtatacgccaatcatggttcgctggaattgggcttggcacttccaacagtctcatgacctgtacttgggctcttgatttctggtatggcggcaaactcatggcagcaggggaaattggagcaaaggcactcttccagaccttcatgattttagttagcactgggcgtgtcattgcggatgctggaacaatgacgaacgacctcgccaaaggtgccgatgcagttgggtcggtcttttcagtgttggatcgatattccttaatcgagccagaggattcagaaggttacaagcccaagaagataacgggcaatgtcgagctgtgtgatgtggatttcgcgtaccctgctaggcccaatgtgattatcttcaaggggttctcaataaaaatcgaagcaggaaaatcaacagcattggtaggacaaagtggatcaggaaaatctactataataggtctaatagaaagattctatgatccctcaagtggtgtagtgaaaatagacggccgtgacataagatcataccacttgagatcattgaggaaacagattgcacttgtaagccaagaaccaacattatttgcaggaaccataagggaaaacatagcctatggagcatcagaagaagtggatgaatcagaaataattgaggctgcgaaggcagcaaacgcccatgatttcatctcagcattaaaagatggatatgagacttggtgtggggacaggggactgcagttgtcaggaggccaaaagcagagaatcgcgatagcacgggcaatattaaaaaatccaggagtgctattgttggacgaggcaacaagtgcattggacagtcaatctgagaaagtagtacaagatgcacttgagagagtgatggttggccgaacaagtgtggtggtggctcacaggctaagtactatacagaattgtgacaccattgctgtactggacaaaggcaaaattgtggagaaagggactcactcttctttgttagccaaagggcctagtggagtttactactctcttgttagccttcaaagaacaccaaactctagcaacacctacatcagttaa

SEQ ID NO: 12 - protein, 1227 aa, Nicotiana tabacum
MSTTTKSKAMIQKKSYGSFVSIFMHADRADILLMILGFFGAVCDGFSMPIMLIVTSKLMNNLGGTDTSNADNFTHHINENALVLVFLACGQWVACFLEGFCWTRTAERQASRLRIRYLKAVLRQDVGYFDLHVASTADVITSVSSDSLVIQDCISEKVPVFLMNVAAFIGSYVVGFLMLWRLALVGFPFVIFLVIPGLMYGRALMGIVRKIRDEYGKAGTVVEQAISSVRTVYSFVGENKTIAEYSVALQGTVELGLKQGLAKGLAIGSNGIVFAIWSFMSYYGSRLVMYNGAHGGTVFAVGAAIAIGGLSLGSGLSNVKYFSEASAAGERVMEVIKRVPKIDSDNMEGKTVALVGGSGSGKSTVVALLQRFYEPLAGEIFVDGVAIEKLQLKWLRSQMGLVSQEPALFATTIKENILFGKEDASMEQVIEAAKASNAHNFISQLPQGYDTQVGERGVQMSGGQKQRIAIARATIKSPRILLLDEATSALDSESERVVQEALDMAAVGRTTIIIAHRLSTIRNADLIAVVQDGQVKEIGSHDDLIEEENGLYTSLVRLQQTEKPSDEFYIAPTNKNIVFAPSNLNPGSASDYDIQNTSSRRLSIVSRSSSANSAAQSRRVDQNATISSTTEQLFPVPSFKRLLAMNLPEWKEATLGCIGAILFGGVQPVYAFAMGSMISVYFLPSHDEIKEKTKIYALCFLGLAFFSLIVNVLQHYNFAAMGEQLTKRVRERMLSKMLTFEIGWYDKDENSTGAVCSRLAKDANVVRSLVGDRMALLIQTVSAVTIACTMGLVIAWKLALVMIAVQPLIIVCYYCKRVLLKSMSKKSIKSQEESSKLAAEAVSNLRTVTAFSSQARILQMLKKAQEGPQRESIRQSWFAGIGLGTSNSLMTCTWALDFWYGGKLMAAGEIGAKALFQTFMILVSTGRVIADAGTMTNDLAKGADAVGSVFSVLDRYSLIEPEDSEGYKPKKITGNVELCDVDFAYPARPNVIIFKGFSIKIEAGKSTALVGQSGSGKSTIIGLIERFYDPSSGVVKIDGRDIRSYHLRSLRKQIALVSQEPTLFAGTIRENIAYGASEEVDESEIIEAAKAANAHDFISALKDGYETWCGDRGLQLSGGQKQRIAIARAILKNPGVLLLDEATSALDSQSEKVVQDALERVMVGRTSVVVAHRLSTIQNCDTIAVLDKGKIVEKGTHSSLLAKGPSGVYYSLVSLQRTPNSSNTYIS

SEQ ID NO: 13 - DNA, 4023 bp, Nicotiana tabacum
  source 1..4023: mol_type = unassigned DNA
tttttcttcccgtgaccacttttctaaggaatagctcctcttcttcttcttgcctttgaagaattttattcttcaaagcttactttcttatctctccattctgttatatctttaaagaatcctacatatagaagtgaacaacaaagatttattaaacaataagggatagcgaaaaggaagaaaaatcctttttctatattcccctcaagattcaagaaaatggccgataatagtggagaaaaaatagaaaaagggtcgagaaaaggagatcaagatcaaaaagtgccattttacaggttattttcgtttgcagataggctggatgttgccctaatgatcgttggaacgataggagccattggcaatggattatctcaacctttaatgacactcatttttggccaacttgttaattcttttggttcttcttctgatgatgatgttgttcacaaaatttcaaaggtttccatttactatgtctacctcgctattgggtctggcattgcatcccttctacaaatgtcatgttggatggttactggagagagacaagcaactcgaattcggggattgtacttgaaaacaattttaaggcaagacattgccttctttgacactgaaacaacaactggagaagtcattggaagaatgtctggagatactattctcattcaagatgccttaggtgaaaaggtcgggaagttcatccaattcctgtcaacatttgttggaggcttcataattgccttcataaagggatggcttctctcaatagtattggtttcttgcatacctgctcttgtcattgctggaggagctatggcattgatcatgtccaaaatgtcaagtcgcggacaagttgcctacgcacaagctggaaatgtagtagagcaaacaataggagcaatcagaacagttgcagcattcacaggagagaagctggcgataagtaagtatgacagcaaactaaaaattgcctgtgctgctactgttcaacaagggcttgtttcaggcgttggacttggcacagtcttactcgtagtcttctctacttatggactagccgtttggtatggttccaaactgataattgagaaaggttataatggtggagatgtcatcagtgttatcatggctataatgactggtggaatgtcactaggccaaacaacaccatcattgaatgcatttgcagcaggacaagctgcagcctacaaaatgtttgagacaatcaaccggaaacctctgattgacacatctgacaccagcgggattgtgttggaagacgtcaagggtgatattgaattgaaagatgtgtacttcagttacccagccagacctgatgagcaaatattcagtggattctcactattcgtatcaagtggcacaactgcagctttggttgggcaaagtggaagtgggaagtcaacagtcataagtctgatagaaagattctatgatcctcaagctggagaggtactcatagatggtgtcaatttgaagaaattccaactcaaatggctaaggcagcagatgggattagtaagtcaggaacctatcctgtttgcaacaaccataaaagagaatatcagttatggtaaagaaaatgctactgacgacgagattaagacagctattgaacttgctaatgctgctaagttcctcaacaaacttccccaggggcttgacactatggtaggtgagcaaggaatacaactatctggtggacaaaagcaaagaattgcaatagcaagggctattctaaagaacccaaaaatactcctactcgatgaggctacaagtgcacttgatgctgaatccgaatgtgtgttacaagaagcactcgacaaagtcatggcgaatagaacaactgtggttgttgctcatcgtctaacaaccattagaaatgctgaccttatagcagtggtgaatgctggaaaactactagaacaaggaacgcattctgagttgatacaagatccaaatggggcgtattcccagcttgtgcgaatgcaaggaggaaatagggaagaagagaacacaaaaaatatggaccttgataaggtggatttaaccacggatttggagaataacttgagcaggtcatcaagccagcgattgtcagcagtgaaacgatcaacaagccacggatcatctagacattccttcacactcagctatcctgttccggggctaattgatattcatgaatcagagataggagatgagggcaagaaaaaagaagataaaggaagcttagagaaacgaaagaaagtttccattaggcggcttgctgaactaaacaaacctgagcttccttatttgctacttggatcgttggctgcgatcatacatggtctgattttccccttatttggactcttgctatcaacagctattaaaatattcttttatccaccacataagttgcgaactgaatcaagattctggtcactcatgtacgttggccttggtgtggtaactttgctagttgtacctttccagaactacttatttggagttgcaggtgggaaattgatcgagagaattcgttctttgacgtttaagaaagtagtccaccaagaaatcagctggttcgatgaccctgcaaattcaagcggtgcagtgggtgcaaggttatcaactgatgcttctacagtcaggacgattatgggtgatgcactggcacttattgtacagaacatagcaactgtagtagctggtcttgtgatagccttcactgctaattggattttggcaattataatccttctcgtattgcctttaattggtttgcaaggattcctccagaccaagttgtacaaaggattcagtgcagatgcaaagatgatgtatgaggaagcaagtcaagttgcaaatgatgctgttggaggtataagaactgttgcgtcattttgtgcagaagagaaggtgatggaaatatacagaagaaagtgtgaaggcccaatgaagcaaggagtaaagataggaattgttagtggagctagctttggttttggttcttttatactgtattgtacaaatgccttctgtttctacataggatctgtccttattcatcatggattagcaacatttggccaagtttttaaggttttctttgcattgacactatcagctgttggagttactcaaagcactggaatggctccagatgctaacaaagccaaggattctatagcttctattttcgacattcttgatagaaaacccaagattgactcaaattctgatgttggcactactctagctgttattcgtggagatattgaatttaaacatgtgagttacagatatgaaactcgcccggatgtccaaatcttcaaggacttatgcctaaccatcccttctggaaagactgttgctctagtgggagagagtggaagcgggaaatcaactgtcattagcctaatagagaggttctacaatcctgaatcaggagagatatatttggatggtgtggagatcaaacaattcaagctaagttggctaaggcaacaaatgggcctagtgagtcaagaaccaatactgtttaacgaaacaattcgtgacaacattgcctacagcagacaagggaatgcaacagaagaagagatcattcaagcagcaaaatcagcaaatgcacacaactttgtatcctcattgcctcaaggatatgatacatctgttggggaaagaggggtacaattatccggtggacaaaagcaaagaatagctatagcgagagcaatactgaaggatccaaagatcctattgctagatgaagcaacaagtgcactagatgcagaatcagaaagtatagtacaagaagcattggatcgagtaatggtaaataggacaaccgtggtagtagctcatcgcttaaccacaatcaaaggggctgatatcattgctgttgtgaagaatggagtaattgctgagaaaggaaggcatgaagttcttatgaacataaaagatggagtttatgcatccttagtagcattgcatatgacttcaacctga

SEQ ID NO: 14 - protein, 1267 aa, Nicotiana tabacum
MADNSGEKIEKGSRKGDQDQKVPFYRLFSFADRLDVALMIVGTIGAIGNGLSQPLMTLIFGQLVNSFGSSSDDDVVHKISKVSIYYVYLAIGSGIASLLQMSCWMVTGERQATRIRGLYLKTILRQDIAFFDTETTTGEVIGRMSGDTILIQDALGEKVGKFIQFLSTFVGGFIIAFIKGWLLSIVLVSCIPALVIAGGAMALIMSKMSSRGQVAYAQAGNVVEQTIGAIRTVAAFTGEKLAISKYDSKLKIACAATVQQGLVSGVGLGTVLLVVFSTYGLAVWYGSKLIIEKGYNGGDVISVIMAIMTGGMSLGQTTPSLNAFAAGQAAAYKMFETINRKPLIDTSDTSGIVLEDVKGDIELKDVYFSYPARPDEQIFSGFSLFVSSGTTAALVGQSGSGKSTVISLIERFYDPQAGEVLIDGVNLKKFQLKWLRQQMGLVSQEPILFATTIKENISYGKENATDDEIKTAIELANAAKFLNKLPQGLDTMVGEQGIQLSGGQKQRIAIARAILKNPKILLLDEATSALDAESECVLQEALDKVMANRTTVVVAHRLTTIRNADLIAVVNAGKLLEQGTHSELIQDPNGAYSQLVRMQGGNREEENTKNMDLDKVDLTTDLENNLSRSSSQRLSAVKRSTSHGSSRHSFTLSYPVPGLIDIHESEIGDEGKKKEDKGSLEKRKKVSIRRLAELNKPELPYLLLGSLAAIIHGLIFPLFGLLLSTAIKIFFYPPHKLRTESRFWSLMYVGLGVVTLLVVPFQNYLFGVAGGKLIERIRSLTFKKVVHQEISWFDDPANSSGAVGARLSTDASTVRTIMGDALALIVQNIATVVAGLVIAFTANWILAIIILLVLPLIGLQGFLQTKLYKGFSADAKMMYEEASQVANDAVGGIRTVASFCAEEKVMEIYRRKCEGPMKQGVKIGIVSGASFGFGSFILYCTNAFCFYIGSVLIHHGLATFGQVFKVFFALTLSAVGVTQSTGMAPDANKAKDSIASIFDILDRKPKIDSNSDVGTTLAVIRGDIEFKHVSYRYETRPDVQIFKDLCLTIPSGKTVALVGESGSGKSTVISLIERFYNPESGEIYLDGVEIKQFKLSWLRQQMGLVSQEPILFNETIRDNIAYSRQGNATEEEIIQAAKSANAHNFVSSLPQGYDTSVGERGVQLSGGQKQRIAIARAILKDPKILLLDEATSALDAESESIVQEALDRVMVNRTTVVVAHRLTTIKGADIIAVVKNGVIAEKGRHEVLMNIKDGVYASLVALHMTST

SEQ ID NO: 15 - DNA, 3847 bp, Nicotiana tabacum
  source 1..3847: mol_type = unassigned DNA
tacgaacctaacaatttccattgtcaccttttttcagctgttggcgtgtagaaacggacctttgagcattgttgaacttgttttagaaaagaaaaagaatgacgacttcatctgaaaaagcattagcgaagaaagatagaaattcagtagctattattttcagatatgctgatggaatagacattttattgatgtgtttgggcactattggagccattggagatggcatatcaacaaactgtttgctggtatatgttagccatctttttaatagcttgggttatggtaagactcaacagaacaatgagatttttatggaagaaattgagaagtgcagcttgtattttgtattattgggattggcagtcatggttgtggcctttatggaaggttactgctggagtaaaacaagtgagcggcaagtgctaaaaattcggtacaagtatttggaagccattcttaggcaagaagttggtttctttgattctcaagaagctaccacatcggaaatcactaacagcatttcgaaagacacttgtctcatacaggaagttcttagtgaaaaggtacccatattttttatgcatacaacagttttcatgtcaggaattgcattttcagcctacttctcgtggaggttggctttagttgccttaccaacacttattctcctcataatcccaggtttgatctatgggaaatatctactccatttgtctgaaaagtcgttcaaggaatatgggaaagcaaatgctattgtatggcaggctcttagctccattaaaactatatattcatttactggtgagaagagcgtgattgaaaggtattcttcgattcttgatcgaaccataatgctgggaatgaagcaaggaattgcaaaaggtcttgctgttggaagcactgggctttcctttgcaatatgggctttactagcatggtatgggagccatttgattatgcacaaaggagaaagtggtggcagaatttatgcagctggagtttcttttgtcttgggtggactatcgcttgggatggcactgcctgaggtgaaatacttgacagaagcttcagttgctgcctcaagaatatttgccaggattgatcgcgttccagaaattgatggggaacacacaaggggactcgttctagaaaatattagaggagaagttgaattcaagaatgtaaacttcacatatccttgtcggccagatagcgttgtgctcaaggaattcaatcttaaaattgaagcaggaaaaacagtggcccttgttggagcaagtggtagtggaaaatccactgccattgccatgattcaaaggttttatgatactaacgttggagctatatgtattgatggtgtagatataaagtcattgcagttgaaatggttgagagggcagatggggttagtaagtcaagaacatgcattatttggaacatctattaagaagaatattatgttagggaaaattgacgcaagcatggatgaagttgtggctgcagcaatgactgcgaatgctcataatttcattacgcaacttccagaaggatatgagaccaagattggtgaaagaggagcatttttatcaggtgggcaaaagcaacgagtggcaatagcgagggccatagtgaaaaatccagcgatacttctactagatgaggccacaagtgcacttgattctgaatcagaaaaactagttcaggaggctctagatcaagcctccattggaagaactacactggttgttgcccacaagttatcaacagttagaaatgcagacctcattgcaatagtgagtagtggttgcatcactgaactagggtcacacaatgagcttatcgagaaagatggacactatggaagaatggcaaaacttcaaagacaatttagctctgttgatcaagaacaaagcgccaaatctcttatttcttcagtaggaagaagcagtgcaggaagacaacgctcaattattagatcaagtccgactgtattcgcctcacctttactcatcgaagacagctcccaagcttcacctcatcctcccccttccttctctcggctccttttactaaacttacctgaatggaagcaaggaataattggaagcttatctgccatttcatttggttcagtgcagcctgtatatgcactgaccataggtggcatgatttcagcattttactcaccaactcacgaggaaatgcaatttagaattcagaaatactgtttgattttcagtatcctttgccttgtttcatttgttctcaatctatgccaacattacaattttgcttacatgggagaacgcctgacaagaagaataagaatgcaaatgcttgagaaaatcttgacgtttgaagcagcctggtttgatgaggaacagaattcaagtggagccttatgttctagattgagcaatgaagcagctatggttaaatcccttgttgctgatagagtctcactcttggtccaaagcatttcagctgtcactgttgccatggtaatggggctagttgtggcttggaagcttgcactagttatgattgctgtccaacctctcacaattctatgcttttacacgcgaaaagtcttgctatccaccatcacagctaagttcgtcaaggcacaatatcaaagcactcaaactgctgtggaggctgtttataatcataggattgtgacctcgtttgggagcataaataaggtccttgaaatctttgatgaggcacaggatgagtcaaggaaggaggctaggaagaaatcgtggttagctggtattggtataggatcagcacaaggcctagctttcatttgttgggccctcgatttttggtatggcggaaagctagttaatgctggagaaatatcagctgctgatgttttcaagactttctttatattagtcagcactggtaaggtagtagctgaagctggaagcatgacttctgatctggccaagggctcagcagcagttgcttctatatttgccattcttgatcgaaactcactcatccatggatcgtatgatgcgaagaataacagtagcggaaccaacttggagaagatgaccggtgggatagagatgaaaaaggttgattttgcatatccaagccggcctaatagcctagtgttgcgcgaattcagcctggaagtgaaagcaggaactagcattggacttgttggaaagagtggctgcggaaaatcaacagtgattgccttgattcaaagattttacgatgcagacaggggatcactgaaaattgatggtatggacattcgattactcgatataggatggtatagaaggcaaatggcgcttgtgagccaagaaccggcaccatccatgaaaacattttgtttggcaaacttgatgcatcagaaaatgaggtcactggataatggatacgatacggaatgtggggagagaggtgtgcagctatcaggagggcaaaagcaaagaattgccattgcaagagcaatattacggaaaccaaccatactacttttagacgaagcaacgagtgctctagatgttcagtcggagcaagttgtgcaggaagcattagatcgactaatggttggaagaacgacagtggttgtggcacacaggcttaataccatcaggaatttagactccattgtatttgtttatgaagggaaggtggtggagaaaggaacctattctcagctaaaggataagcgaggcgcatttttcaatcttgtcaaacttcaatccacataatgtgaagagtgaatgtattcacgtatataagtttcaaattttggacatattgataacttgtgtagtctcaaatggataaaatcattgctttgcatcttccc

SEQ ID NO: 16 - protein, 1246 aa, Nicotiana tabacum
MTTSSEKALAKKDRNSVAIIFRYADGIDILLMCLGTIGAIGDGISTNCLLVYVSHLFNSLGYGKTQQNNEIFMEEIEKCSLYFVLLGLAVMVVAFMEGYCWSKTSERQVLKIRYKYLEAILRQEVGFFDSQEATTSEITNSISKDTCLIQEVLSEKVPIFFMHTTVFMSGIAFSAYFSWRLALVALPTLILLIIPGLIYGKYLLHLSEKSFKEYGKANAIVWQALSSIKTIYSFTGEKSVIERYSSILDRTIMLGMKQGIAKGLAVGSTGLSFAIWALLAWYGSHLIMHKGESGGRIYAAGVSFVLGGLSLGMALPEVKYLTEASVAASRIFARIDRVPEIDGEHTRGLVLENIRGEVEFKNVNFTYPCRPDSVVLKEFNLKIEAGKTVALVGASGSGKSTAIAMIQRFYDTNVGAICIDGVDIKSLQLKWLRGQMGLVSQEHALFGTSIKKNIMLGKIDASMDEVVAAAMTANAHNFITQLPEGYETKIGERGAFLSGGQKQRVAIARAIVKNPAILLLDEATSALDSESEKLVQEALDQASIGRTTLVVAHKLSTVRNADLIAIVSSGCITELGSHNELIEKDGHYGRMAKLQRQFSSVDQEQSAKSLISSVGRSSAGRQRSIIRSSPTVFASPLLIEDSSQASPHPPPSFSRLLLLNLPEWKQGIIGSLSAISFGSVQPVYALTIGGMISAFYSPTHEEMQFRIQKYCLIFSILCLVSFVLNLCQHYNFAYMGERLTRRIRMQMLEKILTFEAAWFDEEQNSSGALCSRLSNEAAMVKSLVADRVSLLVQSISAVTVAMVMGLVVAWKLALVMIAVQPLTILCFYTRKVLLSTITAKFVKAQYQSTQTAVEAVYNHRIVTSFGSINKVLEIFDEAQDESRKEARKKSWLAGIGIGSAQGLAFICWALDFWYGGKLVNAGEISAADVFKTFFILVSTGKVVAEAGSMTSDLAKGSAAVASIFAILDRNSLIHGSYDAKNNSSGTNLEKMTGGIEMKKVDFAYPSRPNSLVLREFSLEVKAGTSIGLVGKSGCGKSTVIALIQRFYDADRGSLKIDGMDIRLLDIGWYRRQMALVSQEPAPSMKTFCLANLMHQKMRSLDNGYDTECGERGVQLSGGQKQRIAIARAILRKPTILLLDEATSALDVQSEQVVQEALDRLMVGRTTVVVAHRLNTIRNLDSIVFVYEGKVVEKGTYSQLKDKRGAFFNLVKLQSTCEEMYSRIVSNFGHIDNLCSLKWIKSLLCIF

SEQ ID NO: 17 - DNA, 471 bp, Nicotiana tabacum
  source 1..471: mol_type = unassigned DNA
atgggaagtgcagtggagacattgtgtggacaagcatatggagcgcagaggtatgagatgctaggaatatacctacaaagatcaacaggaattccactcttattcatctacatcttctccaagccaatcttgattctactcggtcagaaaccagcgatcgcgtcagcagctgcagttttcgtctatggtctaattccccaaattttcgcgtacgcagcgaacttctcaattcaaaaattccttcaatcacagagcattatcaagcctagtgcttatattgcagcagccaccctggttgttcatagcttctttacatggcttgttgtgtataaaactcgttggggaattttgggggcttcattgatgctgagtttttcgtggtggattatagttgtagcacagtttgtgtatattttgtcaagtaaaaagtgcaagaagacatggacaggttggtatgtaatcatggattaa

SEQ ID NO: 18 - protein, 156 aa, Nicotiana tabacum
MGSAVETLCGQAYGAQRYEMLGIYLQRSTGIPLLFIYIFSKPILILLGQKPAIASAAAVFVYGLIPQIFAYAANFSIQKFLQSQSIIKPSAYIAAATLVVHSFFTWLVVYKTRWGILGASLMLSFSWWIIVVAQFVYILSSKKCKKTWTGWYVIMD

SEQ ID NO: 19 - DNA, 1304 bp, Nicotiana tabacum
  source 1..1304: mol_type = unassigned DNA
atgccaccttcacctgaatatgtaacacaaaaatggccagcatcaagcctcatgcaaaatgccgtttcagagctaaaattgcaaagaggaatactacttcctttgatggctatgaacttcacatggtttgcaaaaacagccatcacaactgcatttcttggtaggcttggagatctttatttagctggaggaacacttggttttacttttgcaaatgtaaccggattttcagtactaaatggcctttgtggtgcaatggagcctatttgtggacaagcttttggagccaagaattacaaacttcttcataagacccttgttatggctactttattattactactaatcagcttgcctatttcatttttgtggctaaatattgatacgatcctcattcaatttggccaacaagaagatatttcaaatatggccaaaaagtatctcccatgttactttcaagaactaagggtataataggagtttctatggcatattggataacagattttttgatcatgatatttttgtctatttatgtagtgatagcagagaataaaaagggaggaaaatggaaagaaggaggatggtgggatcaaagaattcgcgattggatcagactactaaaattatgtggaccatgttgtcttactacttgtcttgaatggtggtgttatgaaatcttggttttgttaacaggacatctcccaaatgctaaacaagcaattggagttatagccattgtgttaaattttgattatttgctgttttctgttatgctttcgttatcaacgtgtgcatctattcgtgtgtctaatgagcttggtgcagatagtcctggtcctgcttatcgcgcagcctatgtatcgttagctacgagcattgtttcaggttttcttggtggtgctgttatggctggtgcaagaggaatttgggggccattgtttagtcataataaagggattataaatggcgttaagaaaatgatgttgttaatggcattacttgaagtggttaattttccattagcagtttgtggtggaattgtacgtggaacggctaggccatggctaggaatgtatgctaatattagtggattttatctgttagctttgccattaggtgtggttttggctttcaagattcatcttggtcttactggattgttgacagggtttgtggttggagttgctgtttgtttggccttgttgttggtgtttattgctcggattgattgggttcaagaagctaagaaagcacaagtttttgcttgtaatcttgaagaaataggtaatgatgataattga

SEQ ID NO: 20 - protein, 434 aa, Nicotiana tabacum
MPPSPENVTQKWPASNLMQNALSELKLQKGILLPLMAMNFTWFAKTAITTAFLGRLGDLYLAGGTLGFTFANVTGFSVLNGLCGAMEPICGQAFGAKNYKLLHKSLVMATVLLLLISLPISVLWLNIDTILIHFGQQEDISNMAKKTSPMLFSPAKGIIGVSIAYWITDFLIMIFLAIYVVIAENKKGGKWKEGGWWDQRIRDQIRLLKLSGPCCLTTCLEWWCYEILVLLTGHLPNAKQAIGVIAIYLNFDYLLFSVMLSLSTCASIRVSNELGADSPGPAYPAAYVSLAMSVGSGFLGGAVMAGARGIWGPLFSHNKGIINGVKKMMLLMALLEVVNFPLAVCGGIVRGTARPWLGMYANISGFYLLALPLGVVLAFKIHLGLTGLLTGFVVGVAVCLVLLLVFIARIDWVREAKKAQVFACNLEEIASDDK

SEQ ID NO: 21 - DNA, 2790 bp, Nicotiana tabacum
  source 1..2790: mol_type = unassigned DNA
gatcagtctgtacaatcattggtttcacccctcaggcgcattagctaacgagttttgctcgaaaagtgatcaaaaccagggagtcatggctgaaggtggcaatgtagcttgtactgagagaaaatgcaagataccactctttgttttctttgaagatattaggcatgtgtttaaattcgacgatcttggaatggaaatcctaagtatcgcgttccctgcagctctagcattagctgctgatcccattgcttctctcattgatactgcattcattggtcatttaggttcggtggaaatcgctgcagtaggagtatcaattgccatatttaatcaagcctctaaagtcacaatattccctttagttaacataacaacttctttcgttgctgaggaagacactgttagaagaattaaagaaaaagcagcaattgctgacttggagaaaggttcagaggagaagaatgatacaaaactgccagtaactgatgatactgaccatgagaagctggaaaaatgtgcatccacaaaagttgaaaccaaagagtctgcaccagcaccagcacctgcagcagaattcaagacaacagcatgcaagtctcctgataatcaaagtaaaggcaaagtcaaacgtgtgaagcgacatattccatctgcttcaactgcaattcttatgggctgcattcttggcattttgcaaacaatcttccttatatttctcgcaaaaccaattctaagcttaatgggtgtgaaatctggatctgctatgctttccccagcaaagaagtatttaatactgagagcacttggtgctcctgcagtcctcctttctctggctatgcaaggcgttttccgtggttttaaggatacaagaactcctttatatgctactgttgctggagatttgacaaatatagttttggacccaatctttatctttgttttccgttggggtgttagtggtgctgccattgctcatgtgctttctcagtacttgatatcaattattctcttatgcaaactgatgacagaagtcgaattattacctcctagtgcaaaagatctgcagttcagcaaatttcttaaaaatggattttggttactagcaagggtgatagctgtcacattttgtgtgacgttggctgcgtcattggctgcacgactaggcacaacaccgatggctgcatttcaagtgtgcttacagatctggttaacatcatctctacttgctgatggattggctgtagcaggacaggcaatccttgctagttcttttgctgagaaagactaccagaaggcaaaggctgcaggagtacgagtcctacagatgggatttgtgttgggactaggacttgctttggttgttggaattggtctatattttggatcaggagttttttcaaaggacaaaaatgttatccgtctcataaccattgccatcccgtttgtcgctggtacacaaccaatcaactcattggcgtttgttttagatggtgtcaactttggagcaaatgattttgcatactctgcatattccatggttttggttggtgcactaacagtaacctgtgagtttgtcctttccaaaagcaatggttacattggaatatggattgctttaaccatattcatggccctgcgtaccattgccggtttatggaggatggggacaggaaccggaccctggcgtttcctgcggattccatcatcgtctccagaagccaagtcatagagttcctagcacttctgtggctatttatatcatacattttccatctttctttcctttgtacttctcccttttttcttttgcttttgttgttcttggtgatgcgtacagggtggaaattgtacacactttctactcatatgtttgtataatagtctgacttcagtgtcattatcataagattttgtagctatatgcagatatatacatctttgcatacgtaaactttaattgcttttatattagctagtgccatatcttcatgacataatgttgtacgtttaggcatgcctgtccatcctgacaacgttgcaggtaacatattctccttcatggtgtttatttcgccagtacctacattttatgggatcgttaagaagaaatcaacagaaggctatcaatcaattccgtacgtggttgcactctttagttcaatgctttggatttactatgcatttctcaagacaaacacgacccttatcatcaccataaactcctttggctgcattgcggagactatttatattgctatttattttgcttatgcgacaaaaaaaacaaggatgcaaacgttgagacttgttctaatgttgaatttcggtggctttgggttgattcttttcctcacccaaattttatgcaaaggagcaaaacgagctgaagttattggatggatttgcatggcgttttctattagtgtgtttgtagcacctctaagcattatggggcgggtaatacggaccaaaagtgtggagttcatgccatttaacttgtcgttaacccttacacttagtgctgtgataaacgccaagccaaataatcaggcagaagagaagaaactacccactgttgtgaagctggaggagttgcctacaaaagttaattctgaggtttatccagttagtttaccatccatgggcagtgaaaacggagaggctaaagatggtaaacattttgaagatgcccaaatcaaatctcctattatttacctggaaattaacctaactactgatacaccgaattccggtacaacttgcaagtcagtaagtctagggcaaaagtaa

SEQ ID NO: 22 - protein, 547 aa, Nicotiana tabacum
MAEGGNVACTERKCKIPLFVFFEDIRHVFKFDDLGMEILSIAFPAALALAADPIASLIDTAFIGHLGSVEIAAVGVSIAIFNQASKVTIFPLVNITTSFVAEEDTVRRIKEKAAIADLEKGSEEKNDTKLPVTDDTDHEKLEKCASTKVETKESAPAPAPAAEFKTTACKSPDNQSKGKVKRVKRHIPSASTAILMGCILGILQTIFLIFLAKPILSLMGVKSGSAMLSPAKKYLILRALGAPAVLLSLAMQGVFRGFKDTRTPLYATVAGDLTNIVLDPIFIFVFRWGVSGAAIAHVLSQYLISIILLCKLMTEVELLPPSAKDLQFSKFLKNGFWLLARVIAVTFCVTLAASLAARLGTTPMAAFQVCLQIWLTSSLLADGLAVAGQAILASSFAEKDYQKAKAAGVRVLQMGFVLGLGLALVVGIGLYFGSGVFSKDKNVIRLITIAIPFVAGTQPINSLAFVLDGVNFGANDFAYSAYSMVLVGALTVTCEFVLSKSNGYIGIWIALTIFMALRTIAGLWRMGTGTGPWRFLRIPSSSPEAKS

SEQ ID NO: 23 - DNA, 2179 bp, Nicotiana tabacum
  source 1..2179: mol_type = unassigned DNA
ataaatatgaaactatatcatctaaacaattgtcagttgtgagtgaatgttatgaatgagaaaggtttggctatagtaattgggaagacatggtggaccaaggagctgatgcggataatagaggaaattgcagacttggtgactatgatgtgaaccatggttagaggataggggtaaattagtggagcgggggacaaggaatctggtggttatataattggcagtcttggacgtatttgctatgctgctttaatttggagaacaaagcagcaaaagattagatatggcgatggaagaggcactgctggatgtgaataataataataataataataataataataaggtagtgtttgggaaggaggtgaaggaggtgagtcggatagcaatgccgatgatagtggtgacggtgtcacagtaccttttgcgcgtttcaccaatgataatgttgggtcatcttggtgaacttcaactttctagtgcttctattgctacttcactttccaatgttaccggcttcagtcttatgttagggatggggagtgcagtggagacactatgtgggcaggcatatggagctcacaagtatggaaatcttggaacttttacttatggtgcaattatttgtctattccttgtatgcataccagtttctgttctgtggatcttcacggataggcttcttatattgatgggccaagatcctgaaatcgcaactgaagctggaaaatacacgatttggctcattcctacgctatttccatatgctattcttcagtcacttgtccggtacctgcaggcacagagtttaatcctaccaatgcttttgagtgctgttgtatctttatgcttccaagtgccgatatgttgggtttttatattcaaattggatttggggaacgctggagcagcaatgtcaattggtttatcctattggctgaatgtgatcttgcttatgctttatgtgaagtactcatcagcttgtgcagaaactcgggcttcattctctagagatgtttttctgactataggggacttcttccgctttgctatcccatctgctgtaatggtttgcttggaatggtggtcgtttgaactaatcattctcctctctggtctgttgccaaatccagcgctagagacttccgttctatccatatgctttacaaccacttcagtgcactaccatataccttattctttcggtgccgctgcaagcactcgtgtttcaaatgagcttggagcggggaggccacaggccgcgaaagttgctcttggcgctgtgctaattctttctgtcacagaggttgttcttgcaagtatttctatattcgtggtccgccatgtatggggctatgtatttacctatgagaaagaagtaatcacttatgtagcagaaattactcctgttctttgcatctcaatcatcatggatggcacccaagccgtattatcaggagttgcaagaggaagtgggtggcagcatattggagcatatgtgaatcttggagcatattatttggttggaattccaacggctctattgctgggatttgttttgcatctaaaaggcaaaggcctttggattggattggtggctggagcaactgtgcaatctatttcgctttcccttatcacaggcctcaccaattgggaaaaacaggccattgaagcaagacatagaatctttagtggaaggcctgctgttgaaaatcagaattaaagagcacatgcagatgctataacatacatatcgaaataaaccttcaacttgctatgatttagcgagaatcaccactcatcatagtaccaattggacaattttgaaagtgatcagcctcttagatgtaaatgaacaactttggcggatcaattccagatacggggattttccagtttccttaagcatgttgtttccaagttgaatttgttttgctttgctaaatcgtttagctgcggcttcttgtaacttgtaacgcaaatttcttgttcgaaaatataaacagaaattattgattctgatggtaagcaacccccacttccaaccaagaggttgtgagttcgagtcaccccaagagcaaggtggtagtgtgtacgcagaccttacccctaccctggggtagagaggctgtttccaatagaccctcggcataaatccccaccttgctcttggggtgac

SEQ ID NO: 24 - protein, 479 aa, Nicotiana tabacum
MAMEEALLDVNNNNNNNNNNKVVFGKEVKEVSRIAMPMIVVTVSQYLLRVSPMIMLGHLGELQLSSASIATSLSNVTGFSLMLGMGSAVETLCGQAYGAHKYGNLGTFTYGAIICLFLVCIPVSVLWIFTDRLLILMGQDPEIATEAGKYTIWLIPTLFPYAILQSLVRYLQAQSLILPMLLSAVVSLCFQVPICWVFIFKLDLGNAGAAMSIGLSYWLNVILLMLYVKYSSACAETRASFSRDVFLTIGDFFRFAIPSAVMVCLEWWSFELIILLSGLLPNPALETSVLSICFTTTSVHYHIPYSFGAAASTRVSNELGAGRPQAAKVALGAVLILSVTEVVLASISIFVVRHVWGYVFTYEKEVITYVAEITPVLCISIIMDGTQAVLSGVARGSGWQHIGAYVNLGAYYLVGIPTALLLGFVLHLKGKGLWIGLVAGATVQSISLSLITGLTNWEKQAIEARHRIFSGRPAVENQN

SEQ ID NO: 25 - DNA, 1868 bp, Nicotiana tabacum
  misc_feature 1427..1464: note = n = A,T,C or G
  misc_feature 1582..1621: note = n = A,T,C or G
  misc_feature 1622..1627: note = n = A,T,C or G
  source 1..1868: mol_type = unassigned DNA
  misc_feature 1711..1721: note = n = A,T,C or G
ggaagaggcactgctggatgtgaataataataataataataataataataataaggtagtgtttgggaaggaggtgaaggaggtgagtcggatagcaatgccgatgatagtggtgacggtgtcacagtaccttttgcgcgtttcaccaatgataatgttgggtcatcttggtgaacttcaactttctagtgcttctattgctacttcactttccaatgttaccggcttcagtcttatgttagggatggggagtgcagtggagacactatgtgggcaggcatatggagctcacaagtatggaaatcttggaacttttacttatggtgcaattatttgtctattccttgtatgcataccagtttctgttctgtggatcttcacggataggcttcttatattgatgggccaagatcctgaaatcgcaactgaagctggaaaatacacgatttggctcattcctacgctatttccatatgctattcttcagtcacttgtccggtacctgcaggcacagagtttaatcctaccaatgcttttgagtgctgttgtatctttatgcttccaagtgccgatatgttgggtttttatattcaaattggatttggggaacgctggagcagcaatgtcaattggtttatcctattggctgaatgtgatcttgcttatgctttatgtgaagtactcatcagcttgtgcagaaactcgggcttcattctctagagatgtttttctgactataggggacttcttccgctttgctatcccatctgctgtaatggtttgcttggaatggtggtcgtttgaactaatcattctcctctctggtctgttgccaaatccagcgctagagacttccgttctatccatatgctttacaaccacttcagtgcactaccatataccttattctttcggtgccgctgcaagcactcgtgtttcaaatgagcttggagcggggaggccacaggccgcgaaagttgctcttggcgctgtgctaattctttctgtcacagaggttgttcttgcaagtatttctatattcgtggtccgccatgtatggggctatgtatttacctatgagaaagaagtaatcacttatgtagcagaaattactcctgttctttgcatctcaatcatcatggatggcacccaagccgtattatcaggagttgcaagaggaagtgggtggcagcatattggagcatatgtgaatcttggagcatattatttggttggaattccaacggctctattgctgggatttgttttgcatctaaaaggcaaaggcctttggattggattggtggctggagcaactgtgcaatctatttcgctttcccttatcacaggcctcaccaattgggaaaaacaggccattgaagcaagacatagaatctttagtggaaggcctgctgttgaaaatcagnnnnnnnnnnnnnnnnnnnnnnnnnnnnnnnnnnnnnngaaataaaccttcaacttgctatgatttagcgagaatcaccactcatcatagtaccaattggacaattttgaaagtgatcagcctcttagatgtaaatgaacaactttggcggatcannnnnnnnnnnnnnnnnnnnnnnnnnnnnnnnnnnnnnnnnnnnnnaagttgaatttgttttgctttgctaaatcgtttagctgcggcttcttgtaacttgtaacgcaaatttcttgttcgaaaatatannnnnnnnnnnttgattctgatggtaagcaacccccacttccaaccaagaggttgtgagttcgagtcaccccaagagcaaggtggtagtgtgtacgcagaccttacccctaccctggggtagagaggctgtttccaatagaccctcggcataaatccc

SEQ ID NO: 26 - protein, 477 aa, Nicotiana tabacum
MAMEEALLDVNNKVVVVTRKIFGEEVKEVSRIAIPMIVVTVSQYLLRVSPMIMLGHLGELQLSSASIATSLSNVTGFSLIFGMASALETLCGQAYGAGQYRKLGTFTYGAIICLFLVCIPVSVLWIFTDKLLILMGQDPAIATEAGKYAIWLIPTLFPYAILQSLVRYLQAQSVILPMLLSSVVSLCFQVPICWVFIFKLGLGNAGAAMSIGLSYWLNVILLMLYVKYSSTCAKTRASFSRDVFLTVGDFFRFAIPSAIMVCLEWWSFELIILLSGLLPNPALETSVLSICFTTTSVHYHIPYSFGAAASTRVSNELGAGRPQAAKVALGAVLILSVAEVILASTSIFAVRHVWGYAFTFEKEVITYVAEITPILCISIIMDGTQAVLSGVARGSGWQHIGAYVNLGAYYLVGIPTALLLGFVLHKKGKGLWIGLVAGATVQSISLSLITGFTNWEKQAIEARQRIFSGKHAVENQN

SEQ ID NO: 27 - DNA, 1869 bp, Nicotiana tabacum
  source 1..1869: mol_type = unassigned DNA
ttaaccttttattatctcaaatgaaacctcgagatgacaacttcatctagtatttatatgcctagctagaagttactttcattgcattctacatattcattgtcatgggaaaaagcatgaagtctgaagttgagcagcccttgttggctgctgctcatgggggtagctccgagctagaggaggtgctctccgactcccaattaccctactttcgacgccttaggtatgcctcttggatcgaattccagctactttatcgacttgctgccccttcagttgccgtctacatgatcaacaatgccatgtccatgtctactcggatcttttctggccaactcgggaacctacagcttgcagcagcctctcttggcaatcaaggcatccaattatttgcttatggccttatgctaggaatgggcagtgcagtggaaacgctttgtggccaagcatatggagctcacagatatgaaatgctaggagtctacctgcaaagagcaacagtagtactttccttaacaggcattccactagctgtggtttatttattttccaagaatatactgctcgctcttggtgaatcaaaactagtggcatcagcagcagcagtatttgtttatggtttgattccacaaatttttgcttatgcggtgaacttcccaatacagaagttcttgcaatcccagagtattgttgctcccagtgcctttatttccctggggactctgtttgtacacatattgctcagttgggttgttgtatacaaaattggactgggattgctaggggcgtcgttggtgctgagcttttcttggtggataattgtggtggctcagtttatatatatactaaaaagtgaaaggtgtaaagctacctgggcaggttttcgatgggaggcctttagtggattatgccaatttgtcaagttgtctgctggttcggctgttatgttgtgcttggagacttggtatttccagattctagtgttgctctcgggattactcaagaatcctgagattgccttggcttcaatctctgtttgcttggcagtgaatggactgatgttcatggttgcagtggggttcaatgctgctgctagtgtgagagtgagcaatgagctaggagcagcacacccaaagtcagcagcattctcagtgttcatggtgacattcatttcatttctcattgctgtggtggaagccataattgtgctcagtttgcgcaatgttatcagctatgcattcaccgaaggtgaagttgtggccaaagaagtatctagtttgtgtccatatttagctgtcaccctcattctcaatggcattcaaccagtcttatcgggtgttgctgttggctgtggatggcaggcgtttgttgcctacgtgaatgtagggtgttattatggtgtaggaattccattgggatgtcttctcggcttcaagtttgactttggtgctaagggaatatggactgggatgattggagggactgtgatgcaaaccatcattctgctttgggttacattccgtaccgactggaataaagaggtggagtgcgccagaaaacgtctggacaaatgggaaaacctaaaaggacctctaaacaaggaatgaaggtggtgaagatcattatgacttggaataataatacggggacatgaaattcaaagttattcatgcagtacttgcaagggcaaagaagtcatcaacagtttacatcaatcaaaggttttagcatatctgcgaaattagagagtcacttatcattactttaaatcaattaatcatttcaattctgtcaattcctttgtctgtatctgcacatttattgattgtattacaaagagcttgaaatcaactacagttctccaattaa

SEQ ID NO: 28 - protein, 500 aa, Nicotiana tabacum
MGKSMKSEVEQPLLAAAHGGSSELEEVLSDSQLPYFRRLRYASWIEFQLLYRLAAPSVAVYMINNAMSMSTRIFSGQLGNLQLAAASLGNQGIQLFAYGLMLGMGSAVETLCGQAYGAHRYEMLGVYLQRATVVLSLTGIPLAVVYLFSKNILLALGESKLVASAAAVFVYGLIPQIFAYAVNFPIQKFLQSQSIVAPSAFISLGTLFVHILLSWVVVYKIGLGLLGASLVLSFSWWIIVVAQFIYILKSERCKATWAGFRWEAFSGLCQFVKLSAGSAVMLCLETWYFQILVLLSGLLKNPEIALASISVCLAVNGLMFMVAVGFNAAASVRVSNELGAAHPKSAAFSVFMVTFISFLIAVVEAIIVLSLRNVISYAFTEGEVVAKEVSSLCPYLAVTLILNGIQPVLSGVAVGCGWQAFVAYVNVGCYYGVGIPLGCLLGFKFDFGAKGIWTGMIGGTVMQTIILLWVTFRTDWNKEVECARKRLDKWENLKGPLNKE

SEQ ID NO: 29 - DNA, 1870 bp, Nicotiana tabacum
  source 1..1870: mol_type = unassigned DNA
aacaacctccacgagtccgaagcaccgaattctcttttgtcctccaatggagggctcctctaagcaaccccttctttcgcctaaagaccctcaaatcaccgaccaagctagtgataattatcttatcagattatcacatacttcttttgcttcttcctttgttgctgattccgatgatattcccccgattactggattccgagatttcttcagagagttttctgttgaatctaagaaattatggtaccttgcgggtccagcaatatttacttccctttgtcaatactctcttggtgctgtcactcaaacttttgctggacatgtcgggactcttgagcttgctgccgtttctgttgaaaactcggtcatcgccggtttctcttttggtgtcatgttgggaatgggaagtgcgttagaaacactatgtggacaagcatttggagcaggacaaattgatatgctaggagtatacatgcaaagatcatgggttattctcaatacaacagcagtaattctaatgttactttacattttcgcggcaccatttctgagattaattggacaaacagaggacatatcacgggaagcagggaaaatggctttatatatgattcctcaactttatgcatatgccatgaattttccaatcgccaagttcttgcagtcacagagcaaaattatggtcatggcgtggattgcagcaatagcgttggtattgcatacttttttcagctggttatttatgctaaaacttgggtgggggctcgtcggcgctgcggtggtgctgaactcatcgtggtggttcatagtggtggcgcagctgctatatatatttagtgggacttgtggacaagcttggtcaggtttctcatggaaggctttccataatctatggggctttgttagattatctcttgcgtcagctgtcatgctatgcctggaaacttggtactttatggcgttggtcctcttcgctggttatttaaagaacgcagaagtcgctgttgatgccctgtccatttgtatgaacatattgggatgggcggtgatggcagctattggatgcaatgcagctataagcgtgagagtgtcaaatgaactcggggcagctcatccaagaacggctaaattttcagtggtggtggtggtagtatcctcattcttgattggccttcttctatcggtctttttacttgtctttcgaaggcaatatcctaccttattcgcggagagtgaatcagtcaagcgtcttgtctatgagctaacaccattacttgcattctgcatagtggttaacaacattcagccagctctatctggtgtggcaattggagcaggatggcaagctttggttgcttatgtcaatattgcttgttactatttgtttggtattccactgggtttactattagggtataagctcaatatgggtgtccaaggtatttggtatggaatggttagcggaactctgattcaaactttcgccttattttggatagtttacaaaaccagctggaacaaagaggcttctattgcagcagagaggataaaaatgtggggaggggaatcaaatggaaaggccgatgatgcagagaaatgaactgcgatttggagtcattgcattaagctcatcaagtgtatatgaagaaattttgaaaatttaagttgcttattgttcaatgtaacagctcagtagtactagtgttaatcattgtaaggaactttgccaaaattattctcaacttgaaagtggacaagacccttgataagttagcttttagctaatttttctagtgttttgatatagctttatgccccatttgtcggaattaattgattaattatgctttgaatgatgaagtttcaggat

SEQ ID NO: 30 - protein, 517 aa, Nicotiana tabacum
MEGSSKQPLLSPKDPQITDQASDNYLIRLSHTSFASSFVADSDDIPPITGFRDFFREFSVESKKLWYLAGPAIFTSLCQYSLGAVTQTFAGHVGTLELAAVSVENSVIAGFSFGVMLGMGSALETLCGQAFGAGQIDMLGVYMQRSWVILNTTAVILMLLYIFAAPFLRLIGQTEDISREAGKMALYMIPQLYAYAMNFPIAKFLQSQSKIMVMAWIAAIALVLHTFFSWLFMLKLGWGLVGAAVVLNSSWWFIVVAQLLYIFSGTCGQAWSGFSWKAFHNLWGFVRLSLASAVMLCLETWYFMALVLFAGYLKNAEVAVDALSICMNILGWAVMAAIGCNAAISVRVSNELGAAHPRTAKFSVVVVVVSSFLIGLLLSVFLLVFRRQYPTLFAESESVKRLVYELTPLLAFCIVVNNIQPALSGVAIGAGWQALVAYVNIACYYLFGIPLGLLLGYKLNMGVQGIWYGMVSGTLIQTFALFWIVYKTSWNKEASIAAERIKMWGGESNGKADDAEK

SEQ ID NO: 31 - DNA, 398 bp, Nicotiana tabacum
  source 1..398: mol_type = unassigned DNA
atggttttgccatatctgtcccatcattgaatgaagttctacagatattcatgattgcagcccctgtgtttctaacaatgatgtcaaaggttgcattctactctctacttgtctattttgctacctcaatgggcacacagacaattgctgcgcatcaggtcatggtacaactgttcatgatatgtgcagtatggggtgagcctctctctcaaacagcacagtcgtttatgcccgaattgttatatggagtcaaccgaaatttgtcaaaggctcggatgctgctgaagtcccttttaatcattggagcatcaaatggattattactgggatctgtcggagtatcagttacatggtttttccccaaaatattttcacctgatcctctggtcatacaagag

SEQ ID NO: 32 - skipped

SEQ ID NO: 33 - DNA, 3411 bp, Nicotiana tabacum
  source 1..3411: mol_type = unassigned DNA
gaaatatcaactggattggacagttccactacttactccattgtgaattccttaaggcaatctgttcgaatcttgaagggaacagctgtgatttctctgttgcaacctgcccccgagacctacaacctgttcgatgatattattctgttatccgatgggtatattgtttatcagggtccgcgagatgatgtgctcgagttctttgaatccatgggattcaaatgccctgagagaaaaggcgtggccgacttcttgcaagaagtgacatcaaagaaggatcaacagcaatattggtcaaagaggaatgagccttataggtttatcacatcgaaagaatttgctgaggcatatcagtcttttcatgttggaaggaaactaggcgacgagcttgctactccatttgacaagaccaaatgtcaccctgctgctttgactaatgagaagtatggtataggaaagaaagagctgttgaaggtttgcactgaaagagaacttctactaatgaagaggaactcgtttgtttacatgttcaagttctctcagcttacgataatggctcttataacaatgacactcttcttccgtactgagatgccacgagatactacagatgatggaggaatatatgctggtgctctctttttcgtggtgattatgattatgttcaacgggatgtctgagcttgccatgacaattttcaagcttcccgtcttttacaagcaaagagaccttctctttttcccttcatgggcttatgcaattccctcatggatcctcaaaatccctgtaacacttgttgaagtcggtctttgggtgatcttaacatattatgtcattggatttgatcctaatatttcaagatttttgaaacaattcttgctgctcatagttgtaaaccagatggcatcaggattgtttagattcattggggcagtaggaaggaccatgggagttgctagcacatttggagcatttgctcttcttttacaatttgcattgggcggttttgtcctttcacgagatgatgtgaaaagctggtggatttggggttactggacttcgccgatgatgtattcagtgaattcaattcttgtgaacgaatttgatgggaaaaaatggaatcatattgtgccaggtggaaacgagacccttggatctacagttgtgaagtctcgaggattctttcccgaggcatactggtactggattggtgttggggcacttgtcggatttacagttgtgtttaacttctgctacagtcttgcactcgcttatctcaacccatttgataagccacaagcagtgctaccagaagacggtgagaatgctgaaaatgttgaagtttcatcccagataactagcacagatggaggagattctatcactgagagccaaaataataataagaagggaatggttcttccatttgaaccccattctatcaccttcgatgatgttgtatactccgttgacatgcctcaggaaatgaaagaacaaggtgctggtgaagatagattggtacttctaaagggtgtgagtggagctttcagaccaggtgttctcaccgctttgatgggtgttagtggggctggtaaaacaacattaatggatgttttggctggaagaaaaacaggaggatacattgacggcgacatcaagatttctggctatcccaagaagcaagaaacctttgctcgtatttctggatactgtgagcagaacgacatccattcaccttatgttacagtttacgagtccttggtttactcagcatggcttcgtttaccccaagatgttgatgaaaaaactagaaagatgtttgttgatgaagttatggaacttgttgagcttagacccttaagatcagccttagtcgggttgccaggagtcaacggtctctcaactgagcaacgcaaaaggttgaccattgcagttgaattggtagcaaacccctctatcatttttatggatgaaccaacttcagggctagatgcaagagctgctgccattgtgatgagagctgttaggaacactgtcgatacaggaagaaccgttgtttgtaccattcatcagcctagcatcgacatttttgaagcctttgatgagctatttctaatgaaacgaggaggacaagagatatacgttggtccattgggtcgccattcttgccatttgatcaaatactttgagtcaaatcctggggtagcaaaaatcaaggagggttacaacccagcaacttggatgttagaagtcacagcctcagctcaagaaatgatgttaggcgttgatttcaccgacgtatacaagaactcagacctgtacaggaggaacaaagcattgattagtgaattaggcgtccctcgccctggttcaaaggacttgcattttgaaactcaatactcacaatcattctggacgcaatgtatggcttgcctatggaagcaacactggtcatactggcgtaatccagcttacaccgcagtcagattcatcttcacgacattcatagcactaatcttcgggacaatgttctgggatcttggtaccaaagtgagtaagagccaagatcttttaaacgctatgggatctatgtatgctgctgttctcttccttggtgtacaaaattcatcatcagttcagcccgttgtagccgttgagcgtactgtattttacagagaaagagctgctggaatgtactctgccatcccctatgcctttggacaggtttccattgaaatcccttacatatttgtacaatctgtgttttatggtatcatcgtctatgctatgattggattcgaatgggatgttggcaagttcttttggtacttgttcatcatgtttttcacccttttgtactttacattctatggtatgatgagtgtggctgtcacaccaaatcaaaacgtggcttcaattgttgctgccttcttctatggtgtatggaatctcttctcaggattcatcgttccacgacctcgtatgcctgtatggtggagatggtactactgggctaaccctgttgcatggaccttgtatggtttggttgcatcacaattcggagacatccaaacgacactatctgataatgaaactgtggaacaattcttgagacgttactttggatttaagcatgattttcttggagttgttgcagctgtgctcactgcatatgtctttgtgtttgcctttacatttgcttttgccatcaaggcgttcaatttccagagaagataagtgagtttaatcgactgaagaactttgaggaagcattggttacaaaattagtatgtataggttactacttgagaaagtcttttcttttcaacttttgaatactaggtgtatgttgttgtaatattcattggaaatccaaactttatttgttaatataaaaaaaaaaaaaaaaaa

SEQ ID NO: 34 - protein, 1008 aa, Nicotiana tabacum
MGFKCPERKGVADFLQEVTSKKDQQQYWSKRNEPYRFITSKEFAEAYQSFHVGRKLGDELATPFDKTKCHPAALTNEKYGIGKKELLKVCTERELLLMKRNSFVYMFKFSQLTIMALITMTLFFRTEMPRDTTDDGGIYAGALFFVVIMIMFNGMSELAMTIFKLPVFYKQRDLLFFPSWAYAIPSWILKIPVTLVEVGLWVILTYYVIGFDPNISRFLKQFLLLIVVNQMASGLFRFIGAVGRTMGVASTFGAFALLLQFALGGFVLSRDDVKSWWIWGYWTSPMMYSVNSILVNEFDGKKWNHIVPGGNETLGSTVVKSRGFFPEAYWYWIGVGALVGFTVVFNFCYSLALAYLNPFDKPQAVLPEDGENAENVEVSSQITSTDGGDSITESQNNNKKGMVLPFEPHSITFDDVVYSVDMPQEMKEQGAGEDRLVLLKGVSGAFRPGVLTALMGVSGAGKTTLMDVLAGRKTGGYIDGDIKISGYPKKQETFARISGYCEQNDIHSPYVTVYESLVYSAWLRLPQDVDEKTRKMFVDEVMELVELRPLRSALVGLPGVNGLSTEQRKRLTIAVELVANPSIIFMDEPTSGLDARAAAIVMRAVRNTVDTGRTVVCTIHQPSIDIFEAFDELFLMKRGGQEIYVGPLGRHSCHLIKYFESNPGVAKIKEGYNPATWMLEVTASAQEMMLGVDFTDVYKNSDLYRRNKALISELGVPRPGSKDLHFETQYSQSFWTQCMACLWKQHWSYWRNPAYTAVRFIFTTFIALIFGTMFWDLGTKVSKSQDLLNAMGSMYAAVLFLGVQNSSSVQPVVAVERTVFYRERAAGMYSAIPYAFGQVSIEIPYIFVQSVFYGIIVYAMIGFEWDVGKFFWYLFIMFFTLLYFTFYGMMSVAVTPNQNVASIVAAFFYGVWNLFSGFIVPRPRMPVWWRWYYWANPVAWTLYGLVASQFGDIQTTLSDNETVEQFLRRYFGFKHDFLGVVAAVLTAYVFVFAFTFAFAIKAFNFQRR

SEQ ID NO: 35 - DNA, 4497 bp, Nicotiana tabacum
  source 1..4497: mol_type = unassigned DNA
atggagggaaatgggggaccgagaaggctggggagtagtaggagtagtatgagcagaacgatgagtcgaagcagaagcagagcaaattggaatgtagaagacgtgttcaatccaatgccgagcagaagaagcactcgtggcgaagaagatgaagaagctcttacgtgggctgcgttagagaaattgcctacttacgatcgtttgagaaaaacggtccttaaatcggtcatggaaagtgagaataatcagggcaataaaaaagttgttcataaggaagtcgatgttcggaatctgggaatgaatgagcgacaagagttcattgatcgatttttcagggttgctgaggaagataatgaaaagttcctgagaaagttcagaaatcgaattgacaaagttgggattacccttcctacggtagaagttcggtacgagcatttaactatagaggcagattgctatatcggcgacagagctcttcctacgctgcctaatgctgctagaaatatagctgaatcagctttgagttgcgttggaattaatttggcagagaaaacaaaactaaccatccttaaagatgcttctggaataatcaagccctctaggatgacccttttattaggtccaccatcttctgggaaaacgacccttctgttggctttggctggaaaattagaccctaccttaaaggttagaggagaaatcacttacaatgggcacgggttaaaggaatttgtaccacagaaaacatcggcatacattagtcaaaatgatgttcatgttgctgaaatgactgttaaagaaacccttgatttctctgctagatgccaaggagtcggctctcgatatgaattgctgacggagcttgcaaggagagaaagggatgctggaatcttcccggaggctgaaatcgatcttttcatgaaggcaacagctatggagggagttgaaagcagccttattactgattacacgctcaggattttgggacttgatgtgtgtcgggacaccattgttggggatgaaatgatacgaggcatttctggtggccagaagaagcgtgtcactacaggtgagatgatcgttgggccaacaaaaacactattcatggacgagatatctactggactggacagctccaccacatttcagatagtgaaatgcttgcagcagattgtacatctcactgaggccactgttttgatgtctctcctccagcctgctcctgagacatttgatctctttgacgatatcattcttttatctgaaggccagattgtttaccagggcccacgtgaacatgtcctcgagttctttgaaacttgtggttttaaatgtccggaaagaaagggtactgctgatttcttgcaagaggttacatcaagaaaggaccaagagcagtactgggcaaatagacatagaccataccagtacatttcagtaactgaatttgcaaagagattcaagcgcttccacgtcggcttacgtatagaaaatgagctctctgttccctatgacaaaacaagaagtcacccagcagctctaatattcaagaagtacacagtccctacactagagcttctaaagacaaacttcgacaaggaatggcttttgatcaagagaaactcttttgtttacgtcttcaagaccgttcaaatcatcattgttgcattcattggatcaactgtgttcttgaggaccaaaatgcacactaatactgtagatgatggcgctacctatgttggtgcactcctatttggaatggtcatcaatatgtttaacggtttctctgaactctcaatgatcatacagaggcttcccgttttctacaagcatagggaccttctcttccatccaccttgggctttcactttaccaactgtgctcctaaaggtaccaatttctgtgttcgagactattgtgtggatggtcatgacatactataccattggttatgcccctgaagctagcaggttcttcaagcaatcactgctgacttttctgatccaacaaatggctgctggattatttaggctcactgcaggagtttgtaggactatgattattgcaaatactggtggagcactcatgctccttcttgtgttcctattgggtggtttcatcctgcctagaggctcaattccagactggtggcgatggggctattggatttcacctctttcctatgggttcaatgccttcactgtaaacgaaatgtttgctccaaggtggatgaacaaatttgccccagatggcacaactagattgggtctgcaagtgatgaaaaattttgacgtctttactgaaagacgatggttctggattggtgctgctgctcttctagggttcacaattctcttcaacgttcttttcaccttagtccttatgtatctcagccctttaaacaagccacaagctacactatccaaagagcaggccagcgatatggaagctgatcaagaagaaagcacgggaagcccaagacttaaaatcagccagtcgaagagagatgatctccctcgatccttatctgcagcagatggaaataagacaagagaaatggaaatccgacgaatgagcagtcatatccattctagtggcctctacagaaatgaggatgcaaatcttgaggctgcaaatggtgtcgcagcaaagaaaggaatgatactgccatttacccctctggcaatgtccttcgaggatgtgagctattttgttgacatgccacctgagatgaaggaccaaggagtgacagaggacaaacttcaattgcttcgcgaagtgactggtgcatttaggccaggagtattgacagcattgatgggagtcagtggagcagggaagactacactcatggatgttctagcaggacgtaagactggaggctacatcgaaggtgacgttagaatatctggatttccaaaaaatcaagaaacatttgccagggtttcgggatattgtgaacaaactgatatacactcacctcaagtaactatccatgagtctctgatattttcggctttcctccggctccctaaagaagtcagcaaagaagataagatgatttttgtggatgaagtaatggatctggttgagctagacaatctcaaggatgcaattgtagggcttccaggagttactggtttgtcaacggaacaaagaaaaagattgaccattgcagtagagcttgtcgcaaatccttcaattatattcatggatgaaccgacttctggtctggatgcaagagcagcagctattgttatgagaactgtaagaaacacagtagacaccgggagaactgttgtctgcacaatacatcaaccaagtattgatatatttgaagcatttgatgagctgcttcttatgaaaagaggaggacaagtaatttacgcaggtccattgggtcgacattctcagaaaattattgaatatttcgaggcaattccaggagttcaaaaaattaaagagaaatacaatccagcaacctggatgctagaagcgagttcaattggcacagaagcccgacttggaatggattttgctgaatactacagatcatcagctttacatcaacgaaataaggctcttgtcaaagagttgagtgcacctcctcctggagccaaagacctctattttaccacacaattttcccagccaacatggggtcagtttaaatcctgcttatggaagcaatggtggacttactggagaagtccagactataaccttgtcagattcttctttagtctggctgcagcactattgattgggacaattttctggaatgttggcagtaaaagacaaagtagtggtgatctaatgacagtcatcggggcaatgtatgcagctgtgctgtttgttggaatcaataattgttcgaccgtacagccaattgtagccgtcgaaagaaccgtcttttatagagaaagggctgctgggatgtattcagctttaccatatgctatggcacaggtatttgcagaaataccatacatactcgtccaaactacatactatactcttatagtgtatgctatggtggcctttgagtggacggcagctaaattcttctggttctattttgtaaccttcttctccttcctatactggacgtactatggaatgatgaccgtttccatcacacccaaccaccaagtagccgcgatctttgctgcagctttctatgcacttttcaatcttttctccggtttcttcatccccagaccgagaattcccaagtggtggatatggtattactggatctgtccagtggcatggactgtttatggtagcattgtgtcacagtatggtgatgtggaggatactatccaagtaccgggagtgtttccaaacccaaggattaaggactacattaaagatcatttcggatacaattcggacttcatggcgccagttgctgtggttttggttggttttgcagcctttttcgcctttatgtacgcttacgctattaagacattgaacttccaaactagatag

SEQ ID NO: 36 - protein, 1498 aa, Nicotiana tabacum
MEGNGGPRRLGSSRSSMSRTMSRSRSRANWNVEDVFNPMPSRRSTRGEEDEEALTWAALEKLPTYDRLRKTVLKSVMESENNQGNKKVVHKEVDVRNLGMNERQEFIDRFFRVAEEDNEKFLRKFRNRIDKVGITLPTVEVRYEHLTIEADCYIGDRALPTLPNAARNIAESALSCVGINLAEKTKLTILKDASGIIKPSRMTLLLGPPSSGKTTLLLALAGKLDPTLKVRGEITYNGHGLKEFVPQKTSAYISQNDVHVAEMTVKETLDFSARCQGVGSRYELLTELARRERDAGIFPEAEIDLFMKATAMEGVESSLITDYTLRILGLDVCRDTIVGDEMIRGISGGQKKRVTTGEMIVGPTKTLFMDEISTGLDSSTTFQIVKCLQQIVHLTEATVLMSLLQPAPETFDLFDDIILLSEGQIVYQGPREHVLEFFETCGFKCPERKGTADFLQEVTSRKDQEQYWANRHRPYQYISVTEFAKRFKRFHVGLRIENELSVPYDKTRSHPAALIFKKYTVPTLELLKTNFDKEWLLIKRNSFVYVFKTVQIIIVAFIGSTVFLRTKMHTNTVDDGATYVGALLFGMVINMFNGFSELSMIIQRLPVFYKHRDLLFHPPWAFTLPTVLLKVPISVFETIVWMVMTYYTIGYAPEASRFFKQSLLTFLIQQMAAGLFRLTAGVCRTMIIANTGGALMLLLVFLLGGFILPRGSIPDWWRWGYWISPLSYGFNAFTVNEMFAPRWMNKFAPDGTTRLGLQVMKNFDVFTERRWFWIGAAALLGFTILFNVLFTLVLMYLSPLNKPQATLSKEQASDMEADQEESTGSPRLKISQSKRDDLPRSLSAADGNKTREMEIRRMSSHIHSSGLYRNEDANLEAANGVAAKKGMILPFTPLAMSFEDVSYFVDMPPEMKDQGVTEDKLQLLREVTGAFRPGVLTALMGVSGAGKTTLMDVLAGRKTGGYIEGDVRISGFPKNQETFARVSGYCEQTDIHSPQVTIHESLIFSAFLRLPKEVSKEDKMIFVDEVMDLVELDNLKDAIVGLPGVTGLSTEQRKRLTIAVELVANPSIIFMDEPTSGLDARAAAIVMRTVRNTVDTGRTVVCTIHQPSIDIFEAFDELLLMKRGGQVIYAGPLGRHSQKIIEYFEAIPGVQKIKEKYNPATWMLEASSIGTEARLGMDFAEYYRSSALHQRNKALVKELSAPPPGAKDLYFTTQFSQPTWGQFKSCLWKQWWTYWRSPDYNLVRFFFSLAAALLIGTIFWNVGSKRQSSGDLMTVIGAMYAAVLFVGINNCSTVQPIVAVERTVFYRERAAGMYSALPYAMAQVFAEIPYILVQTTYYTLIVYAMVAFEWTAAKFFWFYFVTFFSFLYWTYYGMMTVSITPNHQVAAIFAAAFYALFNLFSGFFIPRPRIPKWWIWYYWICPVAWTVYGSIVSQYGDVEDTIQVPGVFPNPRIKDYIKDHFGYNSDFMAPVAVVLVGFAAFFAFMYAYAIKTLNFQTR

SEQ ID NO: 37 - DNA, 1566 bp, Nicotiana tabacum
  source 1..1566: mol_type = unassigned DNA
atggagatgaaagtaaacgaagcaatttcaaagagtaaaattggcaaatacttcaagttagaagctagaaaaagctcttttactaaagaatttcgtgctggtacggcgacgtttcttacaatggcttacatcatcaccgtcaacgccagcgttttatcggactccggcggcacttgctccgtttccgactgcacttttccggcgaaccaaacacgtgcaactccagagtgcatgttgaaaccaaatgaaggttatgaaaagtgtgtttccaaaatgaggagtgatcttattgttgctactgctttagcttctatgattgggtcatttgctatgggtttattagctaatcttcctttgggcttggccccaggtatgggcccaaatgcttatttagcttataatttagtgggatttcatgggtctggaaaaatgtcatatcaaactgttatggcaattttcttagttgaaggttgtgcttttcttgctatagctgtatttgggcttcgtgggagaatagcccggtttattcctcagcctgtaagattggcttgtgcggctggtattgggctttttattgcatttgtgggcctacaagcccatcaaggagtgagcctagtcggcccagatccatctactctattgaccctcactgcttgctctagtacaaatccggtgaccggagaatgcaccggcgggaaaatgcaaagcccaactttttggttaagttcagttggatttataattatgtgttatggactaatgaaggaaattaaaggtagtatgatatatggtattttatttgtgacattagtttcttggattaggaatactgctgttacaatttttcctaacacaacatcaggtaattcaagttatgagtacttcaaaaaagtggtagattttcataaaattgagtccacagctggagctattaattttagccattttaataatggtgaagtatgggtggcattattaacattgttatacatagatgtactagctacaacaggtacattatatacaatggctgaaattggtggatttgttaatgaagaaggagaatttgaaggtgaatatatagcatatatggtagatgcaggatcaacaatagttgcatcaactctaggagtttcacctatagccacatttgtagaatcatcagctgggattaaagaaggtggtaggacgggactaacggcgattatcgttggattttattttctattgtctttgttttttacacctttgattgctagtgtaccaccttgggctataggtccatctttggtaatggttggtgtgttgatgatgaaagttgttaaagatattgattggaataatattaagcatgcagtgcctgcatttgtgactatggttctaatgcctttgacatactcaatttccaatggtattattggtgggattggagtgtatattgctctaagtttgtatgatagtatggattgttgggtgaagtggttgatgagaatgagaaagatggtggtgaaggagcaaaatcaagtgtcagctgccactacagatcagaacattgaaattgtttga

SEQ ID NO: 38 - protein, 521 aa, Nicotiana tabacum
MEMKVNEAISKSKIGKYFKLEARKSSFTKEFRAGTATFLTMAYIITVNASVLSDSGGTCSVSDCTFPANQTRATPECMLKPNEGYEKCVSKMRSDLIVATALASMIGSFAMGLLANLPLGLAPGMGPNAYLAYNLVGFHGSGKMSYQTVMAIFLVEGCAFLAIAVFGLRGRIARFIPQPVRLACAAGIGLFIAFVGLQAHQGVSLVGPDPSTLLTLTACSSTNPVTGECTGGKMQSPTFWLSSVGFIIMCYGLMKEIKGSMIYGILFVTLVSWIRNTAVTIFPNTTSGNSSYEYFKKVVDFHKIESTAGAINFSHFNNGEVWVALLTLLYIDVLATTGTLYTMAEIGGFVNEEGEFEGEYIAYMVDAGSTIVASTLGVSPIATFVESSAGIKEGGRTGLTAIIVGFYFLLSLFFTPLIASVPPWAIGPSLVMVGVLMMKVVKDIDWNNIKHAVPAFVTMVLMPLTYSISNGIIGGIGVYIALSLYDSMDCWVKWLMRMRKMVVKEQNQVSAATTDQNIEIV

SEQ ID NO: 39 - DNA, 665 bp, Nicotiana tabacum
  source 1..665: mol_type = unassigned DNA
ggtaacatattctccttcatggtgtttatttcgccagtacctacattttatgggatcgttaagaagaaatcaacagaaggctatcaatcaattccgtacgtggttgcactctttagttcaatgctttggatttactatgcatttctcaagacaaacacgacccttatcatcaccataaactcctttggctgcattgcggagactatttatattgctatttattttgcttatgcgacaaaaaaaacaaggatgcaaacgttgagacttgttctaatgttgaatttcggtggctttgggttgattcttttcctcacccaaattttatgcaaaggagcaaaacgagctgaagttattggatggatttgcatggcgttttctattagtgtgtttgtagcacctctaagcattatggggcgggtaatacggaccaaaagtgtggagttcatgccatttaacttgtcgttaacccttacacttagtgctgtgataaacgccaagccaaataatcaggcagaagagaagaaactacccactgttgtgaagctggaggagttgcctacaaaagttaattctgaggtttatccagttagtttaccatccatgggcagtgaaaacggagaggctaaagatggtaaacattttgaagatgcccaaatcaaatctcc

SEQ ID NO: 40 - protein, 258 aa, Nicotiana tabacum
MPVHPDNVAGNIISFTVFISPVPTFYGIAKKKSAEGFQSVPYVVALFSSTLWIYYAMVKTNETLIITINAFGCIAETIYVAIYFAYATKKTRMQTLRLVLLLNFGGFGLILFLTQILCKGAKRAEVIGWICMAFSISVFVAPLSIMGRVIRTKSVEFMPFNLSLTLTLSAVINAKTNNEAEEKKLPTVVKLEELPTKVNSEVYPVNLPSMGSENGEAKDGKIFEDAQIKSPIIYLEINLTTDTPNSGTTCKSVSLGQK

SEQ ID NO: 41 - DNA, 4212 bp, Nicotiana tabacum
  source 1..4212: mol_type = unassigned DNA
atgtttttataagagaaactaatcacctctttccaaaacaaaagtatatccaatttatctcagagccaaaagtccctttttcatttattatcttctcctcttccttagagtaggggaaaaatagaaagaagagaatggtggaaagtgagaaaatgaatgacacaaagaatctgagcaagagctattttgatgttttgggaatttgctgtacttcagaagttgttcttgttgaaaaaattctcaagaatcttgaaggggttaaagaggtttcagtaattgtcacaacaaagactgtcattgttattcatgattctctcctcatttctcagcaacaaattgttaaagcattgaatcaagcaagattagaagcaagtataagagtgaaaggagagaaaaactaccaaaagaaatggccaagtccatttgcaattggcagtggaatattgcttggactctcatttttgaagtacttttttgcacctttccaatggttagcacttgcagctgttgcagttgggattcctccaattatttttaggggtgtggctgccgtgcgaaacctcactcttgacatcaacattcttgttttaatagcagtgacgggatcaattgttttacacgattattgggaagctggtactattgtcttcttattcaccattgcagaatggctagagtcaagggcaagtcacaaggctactgctgctatgtcatcactggtcaatatagtccctccaacagcagttttagctgaaagtggagaagtcgtaaatgttgatgaagtcaagttgaatagcattcttgctgttaaagctggtgaaactatacctattgatggagttgtaatggaaggggaatgtgacgtggacgagaaaacactgacaggcgagtcgtttccagtttctaagcaaatagattcaacggtctgggctggcactacaaatctaaatgcaattgtggctatatcagcttctttggcaatagttcctactgcattaagagttcacaatcgaaatgaatggtatcgcttggctttggtcacattggtgagtgcatgtccgtgtgcacttgttctatctacaccagttgccatgtgttgcgcactttcaaaagcagcaacgtccggtcttctgtttaaaggagcagagtaccttgagactcttgctaaaatcaaaatcatggcttttgacaaaacagggactataactagaggagaatttatggtgaccgagttcaaggtttcaagtattgagagcaagtcaggtcatccgatggcagccgctctggtggactatgcacaatcaaattccgttgagccaaagcctgatagagttgagcagtttcaaaattttcctggtgaagggatatttggaagaattgatggaatggaaatctatgtcgggaataggaaaatttcttcaagagctggatgtaccacagtaccagaaatagagggtgatagtttccaaggaaagtctgttggatacatatttttgggatcatctcccgctggaattttcggtctttccgatgtttgtcgaattggtgtaaaagaagcaatgagagaactgaagcagatgggtatcaaaaccgcgatgcttactggtgattgttatgcagctgccaaccatgtgcaggatcagaaggaagctccaacagcgatgataggcgacggccttaatgatgctcctgcattagcaacagctgacattggcatctcaatgggcatctctgggtcagctctcgcgaaagaaacaggccatgttatactaatgacaaatgacatcggaagaataccaaaagctgcacgtcttgctagaagagttcgaaggaagattgttgagaatatgattatatcagtcgttacaaaggccgccatagttgcattggcaatagcaggttatccattggtttgggctgctgtcctcgcagatactgggacatgcttgctagtgattttgaacagcatgctacttctacgaggaggcacacgcagacatgggaaaaaatgttggagatcttctactccttcgcatgctccccaccacaaagacaaagcttcatgttgcaagtcggaaaatgctccccagctgtgttgctctgatattgagtcacaaaagaaatgtacgagtcaatcatgctcgtccgaggtgtgtgttccaagatgtcaacctgtctcctcaggatcaaagtcatgtggaaataatcagtgcccagactccgttgaaaatagtggttttcattctcatccccgtcctcaatgctgctcgtcgaagatggctgctaaagcatgccaatctgcagtttcagaatcaaagtcatgtggaaataatcagtgcccagactccgttgaaaatagtgaatcaaagtcatgtggaaataacaattgctcggagtccatttacaagagtagttgtcattctttaacaaactctctagtatgttcttccaagatgtctgctccacaatgtcattctgccacttcaagcaacaaatcatgtggaagtaccaagtgctccgacttcagtgacaaaaaatgttgccaatatgacaaaattcctcaaacgtgctctaccaagaagtctgctccaggatgtcaatctgcagtttctgggtctaaatcatgtggagatagcaagtgttcagactcgaaagacaatagtagccatccttcacatcccgatcatcaaacatgcacgtctaagttgtgtgctccacaaagccaatctgcaacttcaagctccaggacatgtggaaatatgaagtgctcggacaccaatagcaagaattcttgttattcacataccaactctgaatcatgctcttcaaagatgtctggtccatcatgcaaaactgctaattcaggttcaaggtcatgcagaaataagaagtgcctagactctgcaaccgagaacagttttcattcaccacttactaatccactcagtggggaaaagcttttggagaagaaaagcttggatttagtccgaaaagataaggaatcaaatcatgatcttagtcatggttgctctgacgaggaacatgatcatctaaatttagacaaggcacatgacagttgtgccttacaagaatgttgttattctgttcaaggcaataaaactgatgtatcagaaactggaatccaggaagctgctcattgtgacagcatcaatcaaacatgccaaactgcaatttcaggatcaatgacatgcggaaataataagagtctggactctctaagcatccatggttgtcattcgcatgataatccactccacaaggagaacaacttggagcagaaaagcttggatgttgttggagaaggtataaaatcacctcatgctgtcggtcatggctgttcggacaaggaacacgatcactcgcatccagaaaaggcgtatgacagttgtgcaacagacgattgttgtttttcagttcaagtccatggcattgacgacgtatcaagaagtgaaattcaagaaactgctcattgtgacagcacaaaacagagcacggtcatccccagcagctgcaaacatgaaccaaaagatcaggtaaatcactgtggatctcactctaaaagtattccaactgatgaagaactagccaagctggttagaagatgctgcaaatacaaaccatgccacgacgtccgttctggctgcaggaagcatgctgcagaatgtggtccaaccgttcgatcaaccatcaatatcttacgggacaaccatcatcatcatctagactgcagtggtcgtaaggtttgttcgctgttggagaagagacacattggtggatgctgtgacagcttcagaaaagaatgttgtgccaagaacaatcaccttggagcaagttttggaggaggtttatcagaaattgattgggcatcgcttgggctaatgccagttgttagtgtacgtaacgaaggtaagaagaaacttgttatcatagatatatgaaaaatcaactcatggataataaaggcgtagcaccaaatatgtcggagtagccccacccagtagatggaaaagccagtagtaccaatcaaggaagccttgaacggagcaggttgcggtgaattataatgtgcagaattgtggggtataaattagatgattacaattgtggtaaagtgtaattatttattgtactaattctatgtagctatctcccccgaataatggatccactatgagaatttatttagacccg

SEQ ID NO: 42 - protein, 1274 aa, Nicotiana tabacum
MVESEKMNDTKNLSKSYFDVLGICCTSEVVLVEKILKNLEGVKEVSVIVTTKTVIVIHDSLLISQQQIVKALNQARLEASIRVKGEKNYQKKWPSPFAIGSGILLGLSFLKYFFAPFQWLALAAVAVGIPPIIFRGVAAVRNLTLDINILVLIAVTGSIVLHDYWEAGTIVFLFTIAEWLESRASHKATAAMSSLVNIVPPTAVLAESGEVVNVDEVKLNSILAVKAGETIPIDGVVMEGECDVDEKTLTGESFPVSKQIDSTVWAGTTNLNAIVAISASLAIVPTALRVHNRNEWYRLALVTLVSACPCALVLSTPVAMCCALSKAATSGLLFKGAEYLETLAKIKIMAFDKTGTITRGEFMVTEFKVSSIESKSGHPMAAALVDYAQSNSVEPKPDRVEQFQNFPGEGIFGRIDGMEIYVGNRKISSRAGCTTVPEIEGDSFQGKSVGYIFLGSSPAGIFGLSDVCRIGVKEAMRELKQMGIKTAMLTGDCYAAANHVQDQKEAPTAMIGDGLNDAPALATADIGISMGISGSALAKETGHVILMTNDIGRIPKAARLARRVRRKIVENMIISVVTKAAIVALAIAGYPLVWAAVLADTGTCLLVILNSMLLLRGGTRRHGKKCWRSSTPSHAPHHKDKASCCKSENAPQLCCSDIESQKKCTSQSCSSEVCVPRCQPVSSGSKSCGNNQCPDSVENSGFHSHPRPQCCSSKMAAKACQSAVSESKSCGNNQCPDSVENSESKSCGNNNCSESIYKSSCHSLTNSLVCSSKMSAPQCHSATSSNKSCGSTKCSDFSDKKCCQYDKIPQTCSTKKSAPGCQSAVSGSKSCGDSKCSDSKDNSSHPSHPDHQTCTSKLCAPQSQSATSSSRTCGNMKCSDTNSKNSCYSHTNSESCSSKMSGPSCKTANSGSRSCRNKKCLDSATENSFHSPLTNPLSGEKLLEKKSLDLVRKDKESNHDLSHGCSDEEHDHLNLDKAHDSCALQECCYSVQGNKTDVSETGIQEAAHCDSINQTCQTAISGSMTCGNNKSLDSLSIHGCHSHDNPLHKENNLEQKSLDVVGEGIKSPHAVGHGCSDKEHDHSHPEKAYDSCATDDCCFSVQVHGIDDVSRSEIQETAHCDSTKQSTVIPSSCKHEPKDQVNHCGSHSKSIPTDEELAKLVRRCCKYKPCHDVRSGCRKHAAECGPTVRSTINILRDNHHHHLDCSGRKVCSLLEKRHIGGCCDSFRKECCAKNNHLGASFGGGLSEIDWASLGLMPVVSVRNEGKKKLVIIDI

SEQ ID NO: 43 - DNA, 1296 bp, Nicotiana tabacum
  source 1..1296: mol_type = unassigned DNA
atgggaagtgaagctttggagttttttgcttgtgataatacttttcgatatgaaccaaaacgcataacaatggaggagagtgatgatcttcccattaatgatggttcttggtttgaagaagagattgatgttgatctcaaatggtcttttgctttgaacagtgtgctgcacaaaggaacaagcgagtaccaagatatcgcccttttggacaccaagcatttcgggaaggtattggtgatagatgggaagatgcagagtgcagaagtggatgaatttatatatcatgaatgcttaattcatccagctctcttgtgtcaccctaacccaaaaaatgtttttataatgggaggtggtgaaggatctgcagcaagggaagctctcagacacaaatctatggagaaagttgtcatgtgtgacattgatcaggaggttgtggatttctgcaggaagcatctaacagcaaaccatgaggcttttcgtaacaagaagcttaacttgatcattaacgatgccaaagctgagctagagcagaggcaagaaaaatttgatattatagttggagatttagctgatccagttgaaggaggaccttgttaccaactctacaccaaatctttctacgaaaatatccttaaacctaagctcaacgacaatggcatcttcgttactcaggctggaccagcaggggttttcacacacaaggaagttttctcatccatttacaacacaatcaagcaggtcttcaaatatgtgctggcatatacagctcatgtaccctcttttgctgatacatggggatgggttatggcttctgaccaaccattctgtcttgatgctggaaaactggacaagaaaatagctgaaagaattgaaggggaactcttatatcttaatggtgcttctttcttctcttccaccatcttgaataagaccgttgccaaaacgctgaagaatgagactcatgtgtacactgaagatgatgcaaggttcattcatggacatggagtggcattcagaaattgaggcaacaaattggtgggggaactgtacagtcaagtgtttggaaacaaagaaatatgaagtaggggggtgttgaaagttgaaacattgagagttctctcaaagaaagttgttagtagcttttggtttggttcagcaaaataggcttaggcttcttctggttgtcctatgactatacatttactattttatgtactgctcagaaatgaaaaacaaaaaaagacctttttccaatcatatgtcatattttacatgtttggtaaatattattcttat

SEQ ID NO: 44 - protein, 340 aa, Nicotiana tabacum
MGSEALEFFACDNTFRYEPKRITMEESDDLPINDGSWFEEEIDVDLKWSFALNSVLHKGTSEYQDIALLDTKHFGKVLVIDGKMQSAEVDEFIYHECLIHPALLCHPNPKNVFIMGGGEGSAAREALRHKSMEKVVMCDIDQEVVDFCRKHLTANHEAFRNKKLNLIINDAKAELEQRQEKFDIIVGDLADPVEGGPCYQLYTKSFYENILKPKLNDNGIFVTQAGPAGVFTHKEVFSSIYNTIKQVFKYVLAYTAHVPSFADTWGWVMASDQPFCLDAGKLDKKIAERIEGELLYLNGASFFSSTILNKTVAKTLKNETHVYTEDDARFIHGHGVAFRN

SEQ ID NO: 45 - DNA, 1122 bp, Nicotiana tabacum
  source 1..1122: mol_type = unassigned DNA
atggatcatcatcctgctaactatgatcttgatgagaaatctgatctcaaatatgttgattgggtcactttcaagagccattcctccaaaaaagttgagagcttgattctagccaggtttcctaggtgggagtattggaaactttgcctcgtaaacaagagatgttcgatgcttctaaagagcggtgagatatttgagattcgtaaagagattggatttaaagaaccttcagtattcatgttggcaagtggggagactaactggtgggcgtttgatcgagagttcaagtaccggaggaaattgcccgatttaccatcagatgagtgcttcccatttagtgataaggaatcactttgtgtaggcacacatttgcttgtttcgggcagggaaatcgatggtctcgttatttggaggttcgaattagcaacaaatagttggcacaaaggcccctctatggttaatccgaggtgtttatttgcatcagcaacttgtggtaccactgcttttgttgctggtggtgttggtattatgccgaataacgaggtctatgacacagctgagagatacaaccccgatagtagattgtgggagccactaccgaggatgaagaggaatagaaaactttgttcaggatgctacatggacaacagattctacgtgatcgggggaaggaataagaacggtgagttaacgtgtggagaattcttcgacgaggctaagaacaaatgggagctgattccggacatgttgaaggatgatccggtacaggactaccattcaccgcccctccttgctgtcgtgaataacgagctatactcgctcgaggcttcttcgaatcagcttaaggtttacttgaaaaaaaccaacacttggaaacagttagggccagttccagtacgagctgattcaaacaggggatggggcattgcattcaagtcactggggaatgaacttctagtaataggagctgcttcgtcgtcagtgtcttattctggtaattccatggctatatatacttgctgtcctgatcctgaggcaatggagttgcagtggaaaccacttgacagtggtcgaaatcgacttagtagttttatcttaaactgttcagtcatggtagcttga

SEQ ID NO: 46 - protein, 373 aa, Nicotiana tabacum
MDHHPANYDLDEKSDLKYVDWVTFKSHSSKKVESLILARFPRWEYWKLCLVNKRCSMLLKSGEIFEIRKEIGFKEPSVFMLASGETNWWAFDREFKYRRKLPDLPSDECFPFSDKESLCVGTHLLVSGREIDGLVIWRFELATNSWHKGPSMVNPRCLFASATCGTTAFVAGGVGIMPNNEVYDTAERYNPDSRLWEPLPRMKRNRKLCSGCYMDNRFYVIGGRNKNGELTCGEFFDEAKNKWELIPDMLKDDPVQDYHSPPLLAVVNNELYSLEASSNQLKVYLKKTNTWKQLGPVPVRADSNRGWGIAFKSLGNELLVIGAASSSVSYSGNSMAIYTCCPDPEAMELQWKPLDSGRNRLSSFILNCSVMVA

SEQ ID NO: 47 - DNA, 1101 bp, Nicotiana tabacum
  source 1..1101: mol_type = unassigned DNA
actcatggtttcaggtgggagttgttctgagcatgggtgtcaacagcgcctatgctctaggatatgcaggcacaattatggttcctttgggttggactggtggtgtaattggtcttgttttatcaactgttatatcattatatgcaagtactcttatagccaagattcatgaatatgtatgcaaatcttttcttgataaatattggattcatcatttagggggacaagccttaaaggctttctatattctctttagggatgaccatcagatgaagctaccatacttcatcgcgattgctggattagcatgcgtcttatttgccattgctgttcccaccttgtcatcactaaggatttggctggtgatttcatcagtgttcagtctaatttatctcagtatagcatttgcgttgtctcttaaagatggtatgaaggctcctcctagggactatagcattccaggatcaaagataaacagaatttttacaactgctggtgcagctgcaaatattgctacagtaagagcacctgttgttgacaacagtttaggagctgtgcctgttcatgctgttacttacattggatattgggcttatggatccagttcatcttcctatttgctcaacaatgtcagcggtccggtttgggttaaggcactggctaacatcagtgcattcttgcaagctatcatcactttgcatatatttgcaagtccaacatatgagtttctggatacaaaatatggaattaaaggaagtgcagttgctgttcgaaacctggcgtttagaactctagttagaggtggttatatagccattacaactttcctatctgctttgttacctttcctgggagatttcatgagtcttactggtgctatcagctcaattccgctcacgtttatacttccaaatcacatgtaccttgttgccatgaaaaagcaactgtcgtcgttacaaaggagttggcatttgcttaatgttgtcttgtttagtgtcatatctgctgctgcattagtagctgcattcaggcttatagcagtggactcgaaaacttacaacgcatttgctgatttgtagtttttagagtcattatttggag

SEQ ID NO: 48 - protein, 348 aa, Nicotiana tabacum
MGVNSAYALGYAGTIMVPLGWTGGVIGLVLSTVISLYASTLIAKIHEYVCKSFLDKYWIHHLGGQALKAFYILFRDDHQMKLPYFIAIAGLACVLFAIAVPTLSSLRIWLVISSVFSLIYLSIAFALSLKDGMKAPPRDYSIPGSKINRIFTTAGAAANIATVRAPVVDNSLGAVPVHAVTYIGYWAYGSSSSSYLLNNVSGPVWVKALANISAFLQAIITLHIFASPTYEFLDTKYGIKGSAVAVRNLAFRTLVRGGYIAITTFLSALLPFLGDFMSLTGAISSIPLTFILPNHMYLVAMKKQLSSLQRSWHLLNVVLFSVISAAALVAAFRLIAVDSKTYNAFADL

SEQ ID NO: 49 - DNA, 1562 bp, Nicotiana tabacum
  source 1..1562: mol_type = unassigned DNA
gaagaagtggtaaaattgataagtgaaattggagggaaaaatgaacggaggagatatgaatcagcaacagcaacagcagcagcaacaacaacagcagcaacaacaacaacagtggatggcgatgcagcagtatcagcagcaatggatggcgatgcagtatccagcagcagcaatggcgatgcagcaacagatgatgtatggtcagcaatacatgccttacagcaacatcaacaacagcagcagaagatgcagcagtcccctactcagattcagagctcatctgaagataataaaacgatctggattggtgatcttcagcagtggatggatgaaagttatcttcattcttgcttttctcaagctggcgaggttatttctgtgaaaattattcgaaacaagcagactggtcaatcagagcgttatggatttgttgagttcaatactcacgcagcagcggagaaagtgctacagagctacaatggcactatgatgccaaatgcagagcaacccttccgtttaaactgggcaggctttagcactggtgaaaagcgtgcagaaactggttctgatttctcgatttttgtaggagatttggcttctgatgttactgatacaatgttgcgtgacacatttgctagtagatatccatctcttaaaggtgcaaaagtagtagtagatgcaaacacaggccactcaaaaggctatggctttgtaaggtttggtgatgaaagcgagaggtctcgggccatgactgagatgaatggtgtatattgttccagcagggccatgcgtattggtgttgctaccccaaagaaaccatcagcacagcaacaatattcttcacaagctgtgatattatctggtggatatgcatcaaatggtgctgcaacccatggatcccagtctgatggtgattcatcaaacactacaatttttgttggaggacttgattctgatgtcactgacgaggaacttaggcaatcctttaatcagtttggagaagtggtctctgtgaaaatacctgctggaaaaggatgtggttttgtacaattttctgacaggagctctgcacaggaagcgatacagaaattaagtggagcaataattggcaagcaggcagttcgtctttcctgggggcgaagtccagcaaacaagcagatgagaactgattctggtagtcaatggaatgggggttataatggaaggcaaaattatggaggatatggatatggcgcatcgcaaaatcaggattctggcatgtatgctactggagcagcttacggagcatcctctaatggatacgggaatcaccagcagcctgttagctgatctctagtggactctgctgctaaatgagagattgccatagctctgctctctcttccactttgggggatcagatttatatttgacttataagcttaaagcatagtgtttgatgagaattttgtagctggatttattttatgcttagtgagtccaagatatgttaaacctaaacttagaatgattggtgaacttcgatttttctactgcttttttccaaaaaaaaaaaaaaaaaa

SEQ ID NO: 50 - protein, 395 aa, Nicotiana tabacum
MDGDAVSSSSNGDAATDDVWSAIHALQQHQQQQQKMQQSPTQIQSSSEDNKTIWIGDLQQWMDESYLHSCFSQAGEVISVKIIRNKQTGQSERYGFVEFNTHAAAEKVLQSYNGTMMPNAEQPFRLNWAGFSTGEKRAETGSDFSIFVGDLASDVTDTMLRDTFASRYPSLKGAKVVVDANTGHSKGYGFVRFGDESERSRAMTEMNGVYCSSRAMRIGVATPKKPSAQQQYSSQAVILSGGYASNGAATHGSQSDGDSSNTTIFVGGLDSDVTDEELRQSFNQFGEVVSVKIPAGKGCGFVQFSDRSSAQEAIQKLSGAIIGKQAVRLSWGRSPANKQMRTDSGSQWNGGYNGRQNYGGYGYGASQNQDSGMYATGAAYGASSNGYGNHQQPVS

SEQ ID NO: 51 - DNA, 646 bp, synthetic construct
  misc_feature 1..646: note = synthetic oligonucleotide
  source 1..646: mol_type = other DNA
ggatccaaagagcaggccagcgatatggaagctgatcaagaagaaagcacgggaagcccaagacttaaaatcagccagtcgaagagagatgatctccctcgatccttatctgcagcagatggaaataagacaagagaaatggaaatccgacgaatgagcagtcatatccattctagtggcctctacagaaatgaggatgcaaatcttgaggctgcaaatggtgtcgcaggttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggcaactgcgacaccatttgcagcctcaagatttgcatcctcatttctgtagaggccactagaatggatatgactgctcattcgtcggatttccatttctcttgtcttatttccatctgctgcagataaggatcgagggagatcatctctcttcgactggctgattttaagtcttgggcttcccgtgctttcttcttgatcagcttccatatcgctggcctgctcttttctaga

SEQ ID NO: 52 - DNA, 604 bp, synthetic construct
  misc_feature 1..604: note = synthetic oligonucleotide
  source 1..604: mol_type = other DNA
ggatccgttttggtcctttgcataaaatttgggtaaggaaaagaatcaatccaaagccaccgaaattcaacagtaggacaagtctcagtgtttgcatctgcaaaaccattccaaatttcacaatccacgaaactgtgtaacaataactaaaacataacgaaataaatactaggagtataatctataggcacaaaattgaagttgtgcatgttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggatgcacaacttcaattttgtgcctatagattatactcctagtatttatttcgttatgttttagttattgttacacagtttcgtggattgtgaaatttggaatggttttgcagatgcaaacactgagacttgtcctactgttgaatttcggtggctttggattgattcttttccttacccaaattttatgcaaaggaccaaaacgtctaga

SEQ ID NO: 53 - DNA, 603 bp, synthetic construct
  misc_feature 1..603: note = synthetic oligonucleotide
  source 1..603: mol_type = other DNA
ggatccaaagatttcaaggaccttatttatgctcccaaacgaggtcacaatcctatgattataaacagcctccacagcagtttgagtgctttgatattgtgccttgacgaacttagctgtgatggtggatagcaagacttttcgcgtgtaaaagcatagaattgtgagaggttggacagcaatcataactagtgcaagcttccaagcgttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggcaagcttggaagcttgcactagttatgattgctgtccaacctctcacaattctatgcttttacacgcgaaaagtcttgctatccaccatcacagctaagttcgtcaaggcacaatatcaaagcactcaaactgctgtggaggctgtttataatcataggattgtgacctcgtttgggagcataaataaggtccttgaaatctttgtctaga

SEQ ID NO: 54 - DNA, 665 bp, synthetic construct
  misc_feature 1..665: note = synthetic oligonucleotide
  source 1..665: mol_type = other DNA
ggatccgtgtcaacctcttcacttcttcttgctgctcaacttgccttcacggcaataggtgctttcttcatagtgaagctgaaattcacaccctactctatcaatgcagtggttctgttgacagttggtgctgttttattaggtattcgatcaaatggtgatcggccagagggtgtgacaagtagagcttatatttactctttgttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggcaaagaaagataaacctttagtactagtgaggcttgaacgtctgtgacatttaaagtcctaagttagtttctattgtaattgaatataagctctacttgtcacaccctctggccgatcaccatttgatcgaatacctaataaaacagcaccaactgtcaacagaaccactgcattgatagagtagggtgtgaatttcagcttcactatgaagaaagcacctattgccgtgaaggcaagttgagcagcaagaagaagtgaagaggttgacactctaga

SEQ ID NO: 55 - DNA, 600 bp, synthetic construct
  misc_feature 1..600: note = synthetic oligonucleotide
  source 1..600: mol_type = other DNA
ggatccacaccatctaaaacaaacgccaatgagttgattggttgtgtaccagcgacaaactggcgaaggagcacaaggtaaaaagaacagttaaagatagtgaaacaaaagaagaacaaattgaaacaaacatatagtactactatttattgaatgtataccgggatggcaatggttatgagacggataacatttttgtcctttgagttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggcaatcaaaggacaaaaatgttatccgtctcataaccattgccatcccggtatacattcaataaatagtagtactatatgtttgtttcaatttgttcttcttttgtttcactatctttaactgttctttttaccttgtgctccttcgccagtttgtcgctggtacacaaccaatcaactcattggcgtttgttttagatggtgttctaga

SEQ ID NO: 56 - DNA, 687 bp, synthetic construct
  misc_feature 1..687: note = synthetic oligonucleotide
  source 1..687: mol_type = other DNA
ggatccatgggctgatgttcatggtttcaatggggttcaatgctgctgctagtgtaagggtgagcaatgagttaggagcaccacacccaaagtcagcagcattcttagtgtttgtggtgacattcatttcatttctcatagctgtggtggaagccataattatgctgtgtttgcgcaatgtgatcagctatgcattcactaagggttactctttgttctttacttgaacattttaggaatttaggaaatgcttgttcgtcatttgttttgtgtcctagcctattgtttattgtttgtttttatcttcactttagtgaggatacatattctgagcacactctgaaaatatagctcatttatgtttatagggaaaggagaaaagagagagtcacatcatggcaaagaaagataaacctttagtactagtgaggcttgaacgtctgtgacatttaaagtcctaagttagtttctattgtaattgacccttagtgaatgcatagctgatcacattgcgcaaacacagcataattatggcttccaccacagctatgagaaatgaaatgaatgtcaccacaaacactaagaatgctgctgactttgggtgtggtgctcctaactcattgctcacccttacactagcagcagcattgaaccccattgaaaccatgaacatcagcccattctaga

SEQ ID NO: 57 - DNA, 3155 bp, synthetic construct
  misc_feature 1..3155: note = synthetic nucleic acid construct
  source 1..3155: mol_type = other DNA
aagcttccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttgtggatcctgcaggctagcgtgcactctagactcgacgaactgacgagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactatgataaattatcgcgcgcggtgtcatctatgttactagatcgggaattcctcgagcaactatttttatgtatgcaagagtcagcatatgtataattgattcagaatcgttttgacgagttcggatgtagtagtagccattatttaatgtacatactaatcgtgaatagtgaatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaaggttagatgaagtttgtgtatagattcttcgaaactttaggatttgtagtgtcgtacgttgaacagaaagctatttctgattcaatcagggtttatttgactgtattgaactctttttgtgtgtttgcagctcataaaaggtaccaaacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttttgagcgggactctggcgatcgccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactagt

SEQ ID NO: 58 - DNA, 47 bp, synthetic construct
  misc_feature 1..47: note = synthetic oligonucleotide
  source 1..47: mol_type = other DNA
tatgttattcactaaatcatagttaattaatatatatttttacctta

SEQ ID NO: 59 - DNA, 47 bp, synthetic construct
  misc_feature 1..47: note = synthetic oligonucleotide
  source 1..47: mol_type = other DNA
ttcacaactagaagagcgtgagaccttttcaattagaattcgtagga

SEQ ID NO: 60 - DNA, 47 bp, synthetic construct
  misc_feature 1..47: note = synthetic oligonucleotide
  source 1..47: mol_type = other DNA
tccttaaatcggtcatggaaagtgagaataatcagggcaataaaaaa

SEQ ID NO: 61 - DNA, 201 bp, synthetic construct
  misc_feature 1..201: note = synthetic oligonucleotide
  source 1..201: mol_type = other DNA
aattcaaacctgtcaaaaccataaaaagatattggacaaatgcttttaatataattgccttagattaatctatatatatatatatatatatataggtaaatacttacttgtatcagacatttatctttataaatatgttattcactaaatcatagttaattaatatatatttttaccttaaggggccgtttggttgggaaa

SEQ ID NO: 62 - DNA, 201 bp, synthetic construct
  misc_feature 1..201: note = synthetic oligonucleotide
  source 1..201: mol_type = other DNA
tatcgcactactattgaacctatcgccttttgagttttgatatataaatagcgacgaacgtttcttagataatggactcataacctccctcttcacaactagaagagcgtgagaccttttcaattagaattcgtaggaaaaaatcaaacacaaattcacaaaacaaaaatttattaagatttcagcgaccaagcccgtgag

SEQ ID NO: 63 - DNA, 214 bp, synthetic construct
  misc_feature 1..214: note = synthetic oligonucleotide
  source 1..214: mol_type = other DNA
tcgtttgagaaaaacggtccttaaatcggtcatggaaagtgagaataatcagggcaataaaaaagttgttcataaggaagttgatgttcggaatctgggattgaatgagcgacaagagttcattgatcgatttttcagggttgctgaggaagataatgaaaagtttctgagaaagttcagaaatcgaattgacaagtaagtttccagtattact

SEQ ID NO: 64 - DNA, 211 bp, synthetic construct
  misc_feature 1..211: note = synthetic oligonucleotide
  source 1..211: mol_type = other DNA
acaagccacaagctacactatccaaagagcaggccagcgatatggaagctgagcaagaagaaagcacgggaacccctagacttcgaatcagccagtcgaagagagatgatctccctcgatccttatctgcagcagatgggaacaagacaagtatgatctttagcccatcaataacagaatctgcttggggaatataagtaatgcttacagt

SEQ ID NO: 65 - DNA, 200 bp, synthetic construct
  misc_feature 1..200: note = synthetic oligonucleotide
  source 1..200: mol_type = other DNA
atacgataagtcctcttaaaattaccatacttataaagtcataaaagtagaaagaaaaaggacctctttgaaaatttttatataaaaggggctgaaaatatgcgataatgtcaagtagcagtttggcttcatatattggtccatgttatcggagttggtatttatgttaaatattaagtacttttttatcatatctatca

SEQ ID NO: 66 - DNA, 202 bp, synthetic construct
  misc_feature 1..202: note = synthetic oligonucleotide
  source 1..202: mol_type = other DNA
actcaatttctgccactttattataaatagtaagttagtattccattcttggtcagaaaggagtatgggaaatcaaggtctattttcttagttacagacctaacaatttccattgtcaccttttttcagctgttggcgtgtagaaacggacctttgagcattgttgatgcgtttgacttgttttagaaaagaaaaaagaatg

SEQ ID NO: 67 - DNA, 234 bp, synthetic construct
  misc_feature 1..234: note = synthetic oligonucleotide
  source 1..234: mol_type = other DNA
ggctagttgtggcttggaagcttgcactagttatgattgctgtccaacctctcacaattctatgcttttacacgcgaaaagtcttgctatccaccatcacagctaagttcgtcaaggcacaatatcaaagcactcaaactgctgtggaggctgtttataatcataggattgtgacctcgtttgggagcataaataaggtccttgaaatctttgatgaggcacaggatgagtcaa

SEQ ID NO: 68 - DNA, 207 bp, synthetic construct
  misc_feature 1..207: note = synthetic oligonucleotide
  source 1..207: mol_type = other DNA
tttttaaccacctagtggatgctaatatggtgtcagcattagaagaaactaattcatgatttaagttttataggttcaatttttagatttttaatattaaatatattatattttaaagttatgagttaatatttgttgaagtatttgttaagtataattataataaattttaacactaatatttatatttatgctctgcgtcaacag

SEQ ID NO: 69 - DNA, 207 bp, synthetic construct; identical to SEQ ID NO: 68
  misc_feature 1..207: note = synthetic oligonucleotide
  source 1..207: mol_type = other DNA

SEQ ID NO: 70 - DNA, 2544 bp, Nicotiana tabacum
  source 1..2544: mol_type = unassigned DNA
atggaattggagaggggatttccattgctaaaacagcagtacaaagctttattaaagaagaattttatagtggcatggaggaacaagagagccacattccttcagttattctcatccctcttcttgatattccttctttttataatccaaaaagcaatagaagctcgcttttcttcttcctcctcttacgagaacgtgcgggacccacaaccgttagtgtcgccgccaatcccgccctgtgaacaaaagaacttcatccgtctcccctgctatgattttgtttggagtggttctcagagtccaaaaatcgggcagattgcgagcagaattatggctaataatcctggccggtctattcctacctctaaggttctgtcatttagaacacgagatgaagtggatgaatggcttttcaaaaaccctatgcgttgtcctggagctctgcactttgttgaaaggaatgctagcataatcagttatggcatacagacaaactccactcctgttgtcaaacgaggagtctttgaagatccaacttttaaattccaaatcccacttcaactggcagcagaacgtgaaattgcaagatctctgattggagatccaaacttcagttgggttgtcagtttcaaagaatttgcacatcctgcttttgaagttttctcagccttgggtgctgttgggcctacctttttcttggctgttgctatgtttggctttgtcttccaaatcaatgttttgatcattgaaaaggaactcaaacttcggcaggcaatgactatgatgggtctctatgatactgcctactggttgtcatggttcacatgggagggattcatcacacttatctcctctcttctcatagttctctttgggatgatgtttcaatttgagtttttcttgaacaacagttttgccgtcgtgtttctcctttttttccttttccagcttaatatgattggttttgcattcatgctgtctgcttttattagcaagtcatcttcaacaacaactgtgggtttcttcacatttattgtcgatcttcttgctcaaggtcttcagttacttgctgatgcaactgccactcctgaagatcctggtgtcagctggagtggtaggacaaaatgtgcttttaatgataccgagtgtgtaataactatgaatgagatttacgtatggctcgtgtcaacattctttctgtggtttgttcttgctatttacttggacaacataattccgaatgtttctggtgtgagaaaatcaatgttctacttcttgaatcctggatactggacaggcaaaggtggaaataaggtgaaagaggggggtatttgtagctgcacaggttcagtgccgtccctggatagtattataccagatgatgaagatgttcttgaagaagagaacattgttaaacgccaagctacgcaaggtgaagttgattctgatattgcagttcaactacatggccttgtaaagatatttccaggaacagcaaagatgggctgctgtaagtgccaaaggaaatctccttatcatgccctcaagggcttatgggtgaatcttgcaaaggatcagctattttgtcttcttgggccgaacggagctggaaaaactactgctattaattgtttgactgggattacacctgttactgcaggagatgcactagtatatggtcagtctataagaagctctacgggcatgtcaaacattcgaaggatgataggggtttgtccccagtttgatattctttgggatgcattgtccggtcaagaacacctgcatctttttgccagcattaaaggtctacccactgctttagtaaaagaggtcgtagagaagtcactagccgaggtaaaactcacagacgcagccagaatgagagctggtagttacagtggaggaatgaaacgacgcctcagtgttgctatagcacttattggtgaaccgaaattgctcattttggatgaaccgactactggtatggatccaataactagaagacatgtctgggatataattgaggatgcaaaaaaagggcgtgctattatactgaccactcattcaatggaagaagctgacatcttaagtgaccgtgttggtatcatggcaaagggtagacttcgttgcattggaacttcaataagattgaaatcaaggtttggaactggtttcattgctaatgtaagcttttctggtgggacaaacgggactcctgaaacaggagatactttgagtacatctcaacctgaagctgtgaaacagttctttaaaagtcgcttggacgtggtacctaaagaggaaaacaagtccttcttaacctttattattccccatgacaaggagaagttgttgacggacttctttgctgagcttcaagatagagagaaggaatttggcattacagatatccagcttggtcttacaactcttgaagaagtttttctaaacattgctagacaggcagaactggaagatgttgctgaaggaagcttcgcgactcttactttaaattccgggctctcacttcaagtaagttga

SEQ ID NO: 71 - protein, 847 aa, Nicotiana tabacum
MELERGFPLLKQQYKALLKKNFIVAWRNKRATFLQLFSSLFLIFLLFIIQKAIEARFSSSSSYENVRDPQPLVSPPIPPCEQKNFIRLPCYDFVWSGSQSPKIGQIASRIMANNPGRSIPTSKVLSFRTRDEVDEWLFKNPMRCPGALHFVERNASIISYGIQTNSTPVVKRGVFEDPTFKFQIPLQLAAEREIARSLIGDPNFSWVVSFKEFAHPAFEVFSALGAVGPTFFLAVAMFGFVFQINVLIIEKELKLRQAMTMMGLYDTAYWLSWFTWEGFITLISSLLIVLFGMMFQFEFFLNNSFAVVFLLFFLFQLNMIGFAFMLSAFISKSSSTTTVGFFTFIVDLLAQGLQLLADATATPEDPGVSWSGRTKCAFNDTECVITMNEIYVWLVSTFFLWFVLAIYLDNIIPNVSGVRKSMFYFLNPGYWTGKGGNKVKEGGICSCTGSVPSLDSIIPDDEDVLEEENIVKRQATQGEVDSDIAVQLHGLVKIFPGTAKMGCCKCQRKSPYHALKGLWVNLAKDQLFCLLGPNGAGKTTAINCLTGITPVTAGDALVYGQSIRSSTGMSNIRRMIGVCPQFDILWDALSGQEHLHLFASIKGLPTALVKEVVEKSLAEVKLTDAARMRAGSYSGGMKRRLSVAIALIGEPKLLILDEPTTGMDPITRRHVWDIIEDAKKGRAIILTTHSMEEADILSDRVGIMAKGRLRCIGTSIRLKSRFGTGFIANVSFSGGTNGTPETGDTLSTSQPEAVKQFFKSRLDVVPKEENKSFLTFIIPHDKEKLLTDFFAELQDREKEFGITDIQLGLTTLEEVFLNIARQAELEDVAEGSFATLTLNSGLSLQVS

SEQ ID NO: 72 - DNA, 3054 bp, Nicotiana tabacum
  source 1..3054: mol_type = unassigned DNA
atggcggatggtccagcgagcttctggactcaagccaatgctttgctcagaaagaatttgacttttcagatgggtagaaactttttcagcagtggatcaactctaaattcctctgacattttctatagcttagcctataatatcttgggttccgagtcgcagactgagcttatgaattttctcgaggcagctttcttctccaacttgccagtctacaatcttcgacctcaatgtcctccaaactctacattttcttttccattggaatttggttctgtagctgttcagcaagagataagctgtgtgaaaggtatacacttgtggcgtaatagttcttatgagatcaatgatgagctttataaaggttacaggaaaggaaatccagagggaaagataaacgagataatagcagcatatgatttctttaattcaaacagaaatggtttcaatgtgaatatttggtataactctacgtataaggatgacacaggcaatcgacctatgtcattgacaagggttcctcgttcagtgaatttggcatcaaatgcctaccttcagtctttgcttggatcttctgcaagaatgctctttgagtttgtcaaagaaatgcccaaagcagaaacaaaactcaagctggactttgcttctctcctgggaccactattctttacatgggttgttttgatagctctagtttatgagaagcagcagaaactaagaatcatgatgaaaatgcatggacttgcggatggtccctattggatgatttcttattcttattttttggtcgtatcttctatatacatgttatgttttgtggttttcggctcattagtaggcttgaagttctttttgcttaatgattacagcatccagttcgtgttttacttcatttacataaacttgcaaatgtcccttgcttttctggttgctgcgtttttctcgaatgttaagacagctacagtcatcggctatatgatggtgtttgcaaacggactcttggcagcattccttttccagttctttctccaggatgaatcatttcccagaggctggattatagttatggagatatatcctggattttctctttttcgtggattatatgagttttcccaatatgctttcaatgctaattatatgggaacagatggtatgagatggaaagatttgagtgacggaaaaaatgggatgaaggatgtcctaataataatgatagtgcagtggttggtctttctctttcttgcttattacattgatcagattgcatcatcagggaaagatcccctatttttcttgtggaactcccgaaagaagccttcaccttccttcaggaaacatagtttacgaaggcagggttctaaagttttcgtccaaatggagaaacctgatgttgctcaggagagggagagagttgaacagttactcgaatcaagtacaactcatgccatcatttgtgacaatttgaaaaaggtttatccagggaaagacggaaaccctgagaaatttgcagtgagaggattgtcacttgctttgcctcaaggggaatgttttggtatgcttggtcccaacggtgctggcaaaactacttttattaatatgatgattgggctcgtaaaaccaagctccggtactgcatatgctcagggtatggatatacgaaaagacatggatatgatatacaccaacatgggtgtatgtccgcagcatgacttactttgggaaaagttaacaggaagggagcacctacttttctatggaaggcttaaaaatcttaaaggagcagtcctgacacaagcagttgaagaatctcttaagaatgtcaacttatttcatgggggtgttgctgacaagcaagctgggaaatacagtggaggtatgaagaggaggctaagtgttgcaatttcactgatcggagatcctaaggttgtctacatggatgagcccagtactggactggatccagcttcgcgacataacttatggaatgttgtcaagcgtgcaaagcaagatagagcaattattctcacaacccattccatggaagaagcagagcatctatgtgatcgactaggaatattcgttgatggcagcttgcagtgcataggaaatcccaaagaggcactttgggtttgcaaatatcccatatccaattcagcatcagtagctcgatccattcataattttctctcaaaattgaatcactactcttctcttcacatctgttcttcagcgatgataggaacagaagtagcacctactctctctgtcactagatctatcaacagtgggagcaaccttgattccaatcacccttattatcgacattcttctgatgctccagggatgactctagttagcacaccatttgacggaagagaatactcacaatggagcaagtgcaacgacatggtgacctcttggttgctgaattcactcaccaaggagataggagacaatataatttattctagaactgcaaaggatctctgcaacagccttgagcacatgtttggccaatcgagtggagcaaagctctatcatctacagaaggagatctcagagttagctcaaggaagtagtagcatagcatgggcatactgctacaggctcaataagtttcttgatgacttccaatttaccaaatcactcatttctcaaggagcagtgaaggccaatgcattggtttctgcaacaaaaactgatggacaaacaaatggatacactgaaggaaattcaagcaatcaaaatcatttcttcagcaaggaacacgtctctgagctagtgaacataatcaagcaagtacagattggaaatggaggaaatgcaggaacagagatcaatgctaatgcagtagctggtgcctctgagcacatgtgttttgactcaaaatcattcttagaagtccgtccattgcatatacctttgcacataagtttgcctaactcatttcaattgtttgttacacatataggaagggtccctttaatgaggagaggacaagcttttggtgaagtgggggatggtctatacctgcccagcctagtagtttaa

SEQ ID NO: 73 - protein, 1017 aa, Nicotiana tabacum
MADGPASFWTQANALLRKNLTFQMGRNFFSSGSTLNSSDIFYSLAYNILGSESQTELMNFLEAAFFSNLPVYNLRPQCPPNSTFSFPLEFGSVAVQQEISCVKGIHLWRNSSYEINDELYKGYRKGNPEGKINEIIAAYDFFNSNRNGFNVNIWYNSTYKDDTGNRPMSLTRVPRSVNLASNAYLQSLLGSSARMLFEFVKEMPKAETKLKLDFASLLGPLFFTWVVLIALVYEKQQKLRIMMKMHGLADGPYWMISYSYFLVVSSIYMLCFVVFGSLVGLKFFLLNDYSIQFVFYFIYINLQMSLAFLVAAFFSNVKTATVIGYMMVFANGLLAAFLFQFFLQDESFPRGWIIVMEIYPGFSLFRGLYEFSQYAFNANYMGTDGMRWKDLSDGKNGMKDVLIIMIVQWLVFLFLAYYIDQIASSGKDPLFFLWNSRKKPSPSFRKHSLRRQGSKVFVQMEKPDVAQERERVEQLLESSTTHAIICDNLKKVYPGKDGNPEKFAVRGLSLALPQGECFGMLGPNGAGKTTFINMMIGLVKPSSGTAYAQGMDIRKDMDMIYTNMGVCPQHDLLWEKLTGREHLLFYGRLKNLKGAVLTQAVEESLKNVNLFHGGVADKQAGKYSGGMKRRLSVAISLIGDPKVVYMDEPSTGLDPASRHNLWNVVKRAKQDRAIILTTHSMEEAEHLCDRLGIFVDGSLQCIGNPKEALWVCKYPISNSASVARSIHNFLSKLNHYSSLHICSSAMIGTEVAPTLSVTRSINSGSNLDSNHPYYRHSSDAPGMTLVSTPFDGREYSQWSKCNDMVTSWLLNSLTKEIGDNIIYSRTAKDLCNSLEHMFGQSSGAKLYHLQKEISELAQGSSSIAWAYCYRLNKFLDDFQFTKSLISQGAVKANALVSATKTDGQTNGYTEGNSSNQNHFFSKEHVSELVNIIKQVQIGNGGNAGTEINANAVAGASEHMCFDSKSFLEVRPLHIPLHISLPNSFQLFVTHIGRVPLMRRGQAFGEVGDGLYLPSLVV

SEQ ID NO: 74 - DNA, 3315 bp, Nicotiana tabacum
  source 1..3315: mol_type = unassigned DNA
atgtcgcgctgtttttgggaggatgccagcatcattgtctttcttggattcctcggaatcttactgctggattcactgttatgcaaatgcagaaagaaggttatgacagttgatcagaagtacactgttggaacagaagtccgtgtttcctactcctacatattgagcatcatttgcacgactatattatcgtgcactcatctcataatgctcttgatattgcaaaagagaaatggtgctcactgccaattcagatttccagttctctcctctgagattctgcaatcaacttcatgggcagtctcatttttcgtgctctacagaaccaggagtaggaaatacatcaagtttccttgggttctgagaatctggtggatttccagcttctttctgtctattgctcgtgcgactcttgatgcccattttgtcatcacaagcgatgaacatctaggactagcagactatgtggacatcattggtcttatttcatctgcctgtcttctcggtatctcaatccgagggaagacaggcataattcttgacatctcggacagtacaactgagccacttttaaatgggaagaatgaaaaggatccagaagacaaaagggacagtccatatggaaaagctagtcttctccaactgatcaccttctcttggctcaatccactatttgaagttggaaacaagaagccccttgaccaggatgaagtccccgatgttgacttcagggactccgcaaaatttctatctggttcctttgatgaaagcctgaagtacgtaaagggaaggaatggagccaaaaacccatctatctataaggccatttatgtatttgcagggaagaaagcagcaatcaacgctgtctttgcagttattagtgcaggatcatcttatgttggtccataccttatggatgactttgtaaatttcctcaacgaaaagaaacttcgggggttacaaactcgtgcagcttaccaagatgctgatatatatcttcttgatgaccctttcagtgccgttgatgctcacacaggcacgcacctctttcaggagtgcttgaggggggttctcaaggacaagaccatactttatgttacacaccaagttgagtttcttcctgcagcagatctcattctggtgatgcaaaatggaagaattgcacaagctggaacttttgaagaactactgaaacaaaatattggatttgaagttctagttggagcacacaaccaggctttagaatcaattttaacagttgaaagctcaagtagagtatctgaagaagcaattactggtagtgagatggatacagatagtaacataaatacagaaactaagcaggattcagaacacagtctctgtgtagagataacagaaaaagatggaagacttgtgcaggatgaggagagagtaaaaggaagcattggaaaggaagtttactattcttacttgaccaccatgaaaggtggtgcctttgttccaataattcttatagcgcaatcatcatttcaagtgcttcagattgccagcaactactggatggcatcggcattccccacaggtgatgatgcagcaccaatagctgagaagatgaacttcatacttgttgtttttgtgcttctcgctgttggaagttccctttgcgtgctagtgcgggcatcatttgtggctataataggccttcaaacagcagaaaagctctttagcaacatgctgcacagcatccttcgtgctcctatgtcattctttgactctactcctactggacgaatcttaaaccgcgcatccacagatcaaagtgttgtggacttggaaattgcactcaaattgggttggtgtgctctctccattattcagcttcttgggacaattgctgtcatgtcgcaggttgcatgggaagtatttgtcctctttattccaataacagcagtttacgtctggtaccagcaatactacataccaaccgcaagggaacttgctcgtttatctggagttcaaagagctccgatcctccatcactttgcagaatcactggcaggagcagcaacaattcgcgctttcaaccaaaaagatcgctttgctcatgcaaaccttagcctcatagatggcattgcaggattagcagtaacatatggcatctatttgaactattcacaagctgcagtaatatggaatatttgcggtactgaaaacaaaatgatatcagttgaaaggattctccagtattcagaccttgccagtgaagcacccctcgtgattgaaaactgcaggctatcgagcacctggccagaaactggaacaatttccttccaaaatttacagatacgttatgctgaacacctcccttctgttttgaaaaacattacatgcacatttccgggaagtaagaaagttggtgttgtgggaaggacaggaagtggtaaatcaaccctcactcaagcccttttccggatcgtagaacccaaagaaggaagcattatcattgacaatatagatatttgcaagataggtcttcatgatttgaggtcaaggtttagtattattcctcaagatccaacaatgttcgatggaacagttagaggaaacctagatccaatagcacagcactctgatactgaaatctgggaggctctggacaaatgccaacttggtgatataatacgtgcaaagccagaaaagctagaatctacagtggttgaaaacggagaaaactggagtgtgggtcaaaggcagcttttctgtcttggaagagctttgctaaagaaaagtagcattctcgttttggatgaagcaacagcatcagttgatgctgcaactgatgcagtgttacaaaagattatcagtcaagagttcagaaatcgaacagttatcacaatagctcacaggatccatacagtcattaatagcgatcttgtcttagtcttgaatgaaggaagaatagccgaatatgattcaccagcaaagctattggaaagagaggattctttcttctcaaaactgataaaggagtattcgatgagatccaaaggtgcaaaggtgattgagaagcatgcagcacagctttgtgtgttagcagaggactgcgaccagccagattatgtcaaattggtcaaagcactctgtgctgatcacaatgtcagcttgatcacagttccgaatgcaaaaactcttggcgaatgggctggtttgtgtaagattgactctgaagggaaagcaaggaaagtggttggctgcggctgtgttgttgtgaaggattatggggaagagactgagggtctgcatattgtccaggagtacgtaaagtcccattaa

SEQ ID NO: 75 - protein, 1104 aa, Nicotiana tabacum
MSRCFWEDASIIVFLGFLGILLLDSLLCKCRKKVMTVDQKYTVGTEVRVSYSYILSIICTTILSCTHLIMLLILQKRNGAHCQFRFPVLSSEILQSTSWAVSFFVLYRTRSRKYIKFPWVLRIWWISSFFLSIARATLDAHFVITSDEHLGLADYVDIIGLISSACLLGISIRGKTGIILDISDSTTEPLLNGKNEKDPEDKRDSPYGKASLLQLITFSWLNPLFEVGNKKPLDQDEVPDVDFRDSAKFLSGSFDESLKYVKGRNGAKNPSIYKAIYVFAGKKAAINAVFAVISAGSSYVGPYLMDDFVNFLNEKKLRGLQTRAAYQDADIYLLDDPFSAVDAHTGTHLFQECLRGVLKDKTILYVTHQVEFLPAADLILVMQNGRIAQAGTFEELLKQNIGFEVLVGAHNQALESILTVESSSRVSEEAITGSEMDTDSNINTETKQDSEHSLCVEITEKDGRLVQDEERVKGSIGKEVYYSYLTTMKGGAFVPIILIAQSSFQVLQIASNYWMASAFPTGDDAAPIAEKMNFILVVFVLLAVGSSLCVLVRASFVAIIGLQTAEKLFSNMLHSILRAPMSFFDSTPTGRILNRASTDQSVVDLEIALKLGWCALSIIQLLGTIAVMSQVAWEVFVLFIPITAVYVWYQQYYIPTARELARLSGVQRAPILHHFAESLAGAATIRAFNQKDRFAHANLSLIDGIAGLAVTYGIYLNYSQAAVIWNICGTENKMISVERILQYSDLASEAPLVIENCRLSSTWPETGTISFQNLQIRYAEHLPSVLKNITCTFPGSKKVGVVGRTGSGKSTLTQALFRIVEPKEGSIIIDNIDICKIGLHDLRSRFSIIPQDPTMFDGTVRGNLDPIAQHSDTEIWEALDKCQLGDIIRAKPEKLESTVVENGENWSVGQRQLFCLGRALLKKSSILVLDEATASVDAATDAVLQKIISQEFRNRTVITIAHRIHTVINSDLVLVLNEGRIAEYDSPAKLLEREDSFFSKLIKEYSMRSKGAKVIEKHAAQLCVLAEDCDQPDYVKLVKALCADHNVSLITVPNAKTLGEWAGLCKIDSEGKARKVVGCGCVVVKDYGEETEGLHIVQEYVKSH

SEQ ID NO: 76 - DNA, 930 bp, Nicotiana tabacum
  source 1..930: mol_type = unassigned DNA
atggtgtcggatgcggcgaagaagagagatgcacaaaagaaggcggcggcagctgctaggagaggaggaaaaggtgcagccgcgtcatccaagacggtggcgacggcgtccaaagcggcaccggagaccaacggggttgataatttgacgaacggagtgggggatcttcacatatctgatcgaaattgcactggtgttttgtgttcgcatcccctttcaagggatattcggatacaatctttatcagtcaccttccacggacatgatctcattgttgattctgaactggagctcaactatggaagacgttatgggttgctgggactgaatggctgcgggaaatctactcttcttactgctatagggcttcgggaacttcccatcccagagcacatggatatttttcatctttcacgggagattgatgcctctgacatgtctgctcttgaggccgtcattaattgtgatgaagagaggttgaaattggagaaagaagctgaagctttggctggacaggatgatggtggtggagagcaacttgaacgaatctacgagcgtttagaagctatggatgcagccactgctgagaaacgtgctgctgagatcttgtttggtctcggatttgataagaaaatgcaaggaaagaaaacatgtgatttctccggtggctggagaatgaggatttctcttgcgcgagccctatttatgaatccaacaattttacttagaggcctgtgtgtggttagaagaatccttgaagaatttcgagcgcattctggttgtcatttcacactcccaggattttctcaatggtgtttgtaccaatatcatccacatgcaaaacaagaaattgaagctctatacaggaaactacgaccagtatgttcaaacccggtcagagctagaagagaaccagatgaaacagtataa

SEQ ID NO: 77 - protein, 309 aa, Nicotiana tabacum
MVSDAAKKRDAQKKAAAAARRGGKGAAASSKTVATASKAAPETNGVDNLTNGVGDLHISDRNCTGVLCSHPLSRDIRIQSLSVTFHGHDLIVDSELELNYGRRYGLLGLNGCGKSTLLTAIGLRELPIPEHMDIFHLSREIDASDMSALEAVINCDEERLKLEKEAEALAGQDDGGGEQLERIYERLEAMDAATAEKRAAEILFGLGFDKKMQGKKTCDFSGGWRMRISLARALFMNPTILLRGLCVVRRILEEFRAHSGCHFTLPGFSQWCLYQYHPHAKQEIEALYRKLRPVCSNPVRARREPDETV

SEQ ID NO: 78 - DNA, 1296 bp, Nicotiana tabacum
  source 1..1296: mol_type = unassigned DNA
atggagttacaggttaatgttccaagatggacacctagtccaaacagatcaccacatagacgccaagaatctgagacagataatgaaagtgttacttattctcaagatgatattccttttaacaacaaaaacccaaccaaaaatttcccatttagtaatagtccaccacctcatattgctgctattgaagcaccatctcttagggttgattctgaaataaaaagggaagagattaagggaaataataatactactggttttgcatatgaggaagttgtactcccttttaataatgaaggaatttacttgacatggaaagatttgtgggtgacagtgccagacaagaaaacagggaggagagcaatattacaagggctaactggttatgtacaacctggtcaagttttggccattatgggtccttctggttgtggcaaatccactcttcttgatactttagcagggagattggattcgaacacgagacaaactggagaaatcctcatcaatggtcggaggcaagctcttgcttttggcacttcggcttatgtgacacaagatgatacattgatgacaacactgacagtgaaagaagcaatatactattcagcacaactccaattaccagattcaataatagttcaattggctaaacaagatggaagaactgtggttgcttctatacatcagccaagtagtgaagtttttgagcttttccacaatctttgccttctctcctctggtaggactgtctactttggttccatttctgctgcaaatgagttttttgcactgaatggcttcccatgtccaactatgaggaatccttctgaccactacttaaggacaatcaacaaggactttgatgctgatatcgagaaaggagttggtggaaaagccacggcgacagaagcaatcaatattctggttaagtcatacaagacctcacagggttgccaacaagttcaacgtcgagttctggagatttgccaacagaatggtggacaagaagcaaagaaaggaaaccaagcaagtttcattactcagtgcacggttctaaccaggagatcttttgtgaacatgtatcgtgatcttggttattactggcttcgttttgcaatatatattgctttgtgcttgtgtgttggcactatatttcatgacattggccacgactatggctccattcagttccggagttatgatgaaaatcgtgttctatcgtatattggcatgttgggtgcagtgagtagtatggaatttgaagtgaggatcaatgttgcagttcggtgttga

SEQ ID NO: 79 - protein, 431 aa, Nicotiana tabacum
MELQVNVPRWTPSPNRSPHRRQESETDNESVTYSQDDIPFNNKNPTKNFPFSNSPPPHIAAIEAPSLRVDSEIKREEIKGNNNTTGFAYEEVVLPFNNEGIYLTWKDLWVTVPDKKTGRRAILQGLTGYVQPGQVLAIMGPSGCGKSTLLDTLAGRLDSNTRQTGEILINGRRQALAFGTSAYVTQDDTLMTTLTVKEAIYYSAQLQLPDSIIVQLAKQDGRTVVASIHQPSSEVFELFHNLCLLSSGRTVYFGSISAANEFFALNGFPCPTMRNPSDHYLRTINKDFDADIEKGVGGKATATEAINILVKSYKTSQGCQQVQRRVLEICQQNGGQEAKKGNQASFITQCTVLTRRSFVNMYRDLGYYWLRFAIYIALCLCVGTIFHDIGHDYGSIQFRSYDENRVLSYIGMLGAVSSMEFEVRINVAVRC

SEQ ID NO: 80 - DNA, 3855 bp, Nicotiana tabacum
  source 1..3855: mol_type = unassigned DNA
atgaagacatattcaacagcagggacttcagtttacattattcagacagataatcatattccagagcttatcgtaagagaaacagtagatttcgcagctagatgccaaggtgaaaatcacaattttgaagactatgtcttgaatgttcttcgacttgatatatgttcagacacattagttggtaatgacgcgataagaggagtttcaggtgaacaaaggaagataggagaaatgattgttggtcccaggaaaacactgttcatggatgagatttctaccggacatgacagtagtacaagttccaaaatagtcaagtgcatacaaaattttgttcatataatggaaggaactgtgatgatggctctttgtcgtccttcacaagagactttcgaattgttcgatgatcttgtattactatctgaaggatatgtcctgtaccatggccctcgagaagatgttattccattttttgagtggttaggatttcaattgccagatcgtaaggatgtcgttgattttattcaagaggttgcatttgtcggatccgtcacaggcacaatgtttctaagaacaaggttacatcccacagatctggtgaacgggaacttgtatctttcctgcttgtttttcggtctggttcatatgatgctcgacggattctcagaactgtctctctttatatatcggctcccagtattctataagcaaagagataatttcttctatcctgcgtgggtgtggtcattgtgtagttggatcttgtcgttaccttactctgttattgaagctcttgtatggtcttatgttgtatactggactgtaggatttgcacctggtgctgggagatttttcagctacatgtttttactcttctcggtacaccaaatggcgatgggtctctttcggttaatagcttctttgtcacgagatataattattgcaaatacttttggatcagctgtgctaatcatatttttactgggtggttttatcttgccaaaagaaatgatcaagccatggttcgtatgggcattttgggtgtcaccattgtcgtatggacagcgagcaatttctgttaatgaattcactgcgacaagatggattgagaagacaaccagcggaaatgtcacgcttggttacagtgttctgcaatcgcacagcctaccgacatctggttactggtactggttgggattgggagttctgttgctctctgtgttgcttctcaacattattctgactgtagccttgacttttctcaacccactaagaaaatctagggcaattattccaacagatgccagtggtgtaaattcagttcctggtggaaatagcaaccgtggccagggggggactagacagtcagaattgcgcctacctttcaaacaagaaatgggaataccgcagaaaaggttgcagctactgtccaatgttagtagagtattatcacctggcgttcttattggattggttggtgcaagtggagcggggaaaaccacattgatggattgcctagctggtaggaagactactggatatgtagagggtgatattaggatatcaggctacccaaaaaaacaagagacttttgcgagaatttcaggatatgttgaacagaatgatatacattctcctcaagtgacagtttttgaatccctatgttttacgtcttatctccggctctccaaaaaagtgaatgaaaaacaaagactggagtttgttgaagaaataatggcattactggaacttgactctctaaggcatgctttagttggcttgcctggcatttctggcttatcaaccgaccaaagaagacgcttgacaattgcagtagaacttgtagcaaatcctgcgatactttttatggatgatcctacatctggtcttgatgcacgagctgcttctattgtcatgcgaactgttagtgatgtagcacatggcggaagaattgtggtctgctctattcgccatccaagttttgatatttttgaaacatttgacgagcttcttctaatgaaacacgggggacaactgatatatggaggacagcgtgggaacaattcacaaactatgattaactatttccagggtattgatgggatacccccaattcctaatggttgcaatcctgcaacgtggatgcttcagataagtacaccagctgctgaagcaagaataggacgagattttgctgaaatatacagaagttctgcactctacagggaagttgaagccagaactatgcttccaccagcagaaaactcaaaacctctgaggttcacttccaaatataacacatctgtactttatcagcttaaaatgtgcctgtggaagcaaagtcttgtatactggagaaatccagcacattataattgtgagattcttctccacaactttttatttcttggggtccagaattcttcctccatacagcccattgtttcaatgggaagacccgttttccgtagggagagagatgcagggatgtactcctgcgtagtatatgcagcatctcaggggctgattgaggttatttatgtcctggcccagactgtactatttggagccattacctattctatgttgggcttagtgagggcagcaggcacgattttcatttacatcatgttcttgcttctgaccacctattttaccttttacggtatgatggtggggtatgacttggttctatgtatcagcttaagaatttctggaatttggcggattgaattgctgtccattgctatcgagttgctctcgtttggtcatcggtttgttgctgttatgttgccggatctcctcgcctatttcagaagcttgcttgttgtttttcttcagttaatttcggctgctagttactttcggtgctacaccagtggtagcggtgatagccctttttggtctttgggtcgtggtgttttctgtgttttcagggaaagtctcaaagtggttggagacaagttgggtgcacgagcactagcaaaggagagcaacctggacgagcgtcaacaaagggcggtgggaagcagtgtagtggtggcagtctgggatgatagattaaaggcatgtcctcagaggtcagtacgaggccgcccgaggattaggtggggctccttgactaaggttaaagcccaggagttggaaggaaggttgtcggcaatgggagcttggagaagtagtgaggacgcaaacactatgtggtcaacgacggcagactatataaggaaggcggcgagagaggtgttaggggtatcttcaggccgcacctgtggccacaaaggagactggtggtggaatgcagttgtacaaggtaaagtggaagcgaagaaggtggcttacctgcggttagtggggagcactggagaggaggagaagagagcgaacattgcgagatataaggtagctaggaaggaggcaaagatggcagtgacggaggcaaagacgacagcttttgctcgtttgtatgaggaactagggaacaaaggcggggagaagaagttactccgactcgctaaggtgagagagaggacggctcgggatctggacctagtgaggtgcataaaagatgaggacgacaaagtttttttgggggatgaccagataaagaggagatggcagacctactttcataaacttctaaatgaggaaggggatcaggatattgtattaggtgaattgaggaacgacgacaacccccatgaactaagtgattgtcgggacattgaggtcgatgaggtcatggaggcaatgcaaggagatgaaaattgtcggagaaagtttttcggtggtattaattgggtgaaggcagaaattcatgagttttgtcaagtgaaattggtggtgaaaatagagtag

SEQ ID NO: 81 - protein, 1284 aa, Nicotiana tabacum
MKTYSTAGTSVYIIQTDNHIPELIVRETVDFAARCQGENHNFEDYVLNVLRLDICSDTLVGNDAIRGVSGEQRKIGEMIVGPRKTLFMDEISTGHDSSTSSKIVKCIQNFVHIMEGTVMMALCRPSQETFELFDDLVLLSEGYVLYHGPREDVIPFFEWLGFQLPDRKDVVDFIQEVAFVGSVTGTMFLRTRLHPTDLVNGNLYLSCLFFGLVHMMLDGFSELSLFIYRLPVFYKQRDNFFYPAWVWSLCSWILSLPYSVIEALVWSYVVYWTVGFAPGAGRFFSYMFLLFSVHQMAMGLFRLIASLSRDIIIANTFGSAVLIIFLLGGFILPKEMIKPWFVWAFWVSPLSYGQRAISVNEFTATRWIEKTTSGNVTLGYSVLQSHSLPTSGYWYWLGLGVLLLSVLLLNIILTVALTFLNPLRKSRAIIPTDASGVNSVPGGNSNRGQGGTRQSELRLPFKQEMGIPQKRLQLLSNVSRVLSPGVLIGLVGASGAGKTTLMDCLAGRKTTGYVEGDIRISGYPKKQETFARISGYVEQNDIHSPQVTVFESLCFTSYLRLSKKVNEKQRLEFVEEIMALLELDSLRHALVGLPGISGLSTDQRRRLTIAVELVANPAILFMDDPTSGLDARAASIVMRTVSDVAHGGRIVVCSIRHPSFDIFETFDELLLMKHGGQLIYGGQRGNNSQTMINYFQGIDGIPPIPNGCNPATWMLQISTPAAEARIGRDFAEIYRSSALYREVEARTMLPPAENSKPLRFTSKYNTSVLYQLKMCLWKQSLVYWRNPAHYNCEILLHNFLFLGVQNSSSIQPIVSMGRPVFRRERDAGMYSCVVYAASQGLIEVIYVLAQTVLFGAITYSMLGLVRAAGTIFIYIMFLLLTTYFTFYGMMVGYDLVLCISLRISGIWRIELLSIAIELLSFGHRFVAVMLPDLLAYFRSLLVVFLQLISAASYFRCYTSGSGDSPFWSLGRGVFCVFRESLKVVGDKLGARALAKESNLDERQQRAVGSSVVVAVWDDRLKACPQRSVRGRPRIRWGSLTKVKAQELEGRLSAMGAWRSSEDANTMWSTTADYIRKAAREVLGVSSGRTCGHKGDWWWNAVVQGKVEAKKVAYLRLVGSTGEEEKRANIARYKVARKEAKMAVTEAKTTAFARLYEELGNKGGEKKLLRLAKVRERTARDLDLVRCIKDEDDKVFLGDDQIKRRWQTYFHKLLNEEGDQDIVLGELRNDDNPHELSDCRDIEVDEVMEAMQGDENCRRKFFGGINWVKAEIHEFCQVKLVVKIE

SEQ ID NO: 82 - DNA, 861 bp, Nicotiana tabacum
  source 1..861: mol_type = unassigned DNA
atgggagataacaaccatcaccaagtgtttgatgtatccgccaatggctacggtgaatccaagtgttttgacgatgatggtcgtcttaaaagaagtggtagtgtttggactgcaagtgctcatataatcacagcagttattggttctggagttttgtcattagcttgggccactgctcagcttggttgggttgctggtccagctgttttgcttctcttctcctttgttacttactacacttctgcgctcctcgctgattgttaccgttctggagatcaagtttctggcaaaagaaactatacttatatggatgctgtccgagccaatcttggtgggcttcaggtgaagatttgtggggtaattcagtatgtgaatctttttggagttgcagttggctatactattgcatcttctattagcatgatggctgtgaaaaggtctgattgtttccataagcatggacacagagcagcttgcaatgtttcaagcactccatacatgattatgtttggagtaatggaaatcctcttctcacaaatcccagattttgatcagatttggtggctttctattgtggctgctgttatgtctttcacatactctaccattggtcttggtttaggagttgcccaagttgcagaaacaggaaaaatccaaggaagtctcactgggattagtattggaacagaagtaactgaaatgcagaagatttggagaagctttcaagctcttggagctattgcttttgcttattcttactccctcatccttattgagattcagtctttgactgtgtttgatcatgtagatattgccttgagtggtttggatcacgttgaaagtgttttggcttttggttga

SEQ ID NO: 83 - protein, 286 aa, Nicotiana tabacum
MGDNNHHQVFDVSANGYGESKCFDDDGRLKRSGSVWTASAHIITAVIGSGVLSLAWATAQLGWVAGPAVLLLFSFVTYYTSALLADCYRSGDQVSGKRNYTYMDAVRANLGGLQVKICGVIQYVNLFGVAVGYTIASSISMMAVKRSDCFHKHGHRAACNVSSTPYMIMFGVMEILFSQIPDFDQIWWLSIVAAVMSFTYSTIGLGLGVAQVAETGKIQGSLTGISIGTEVTEMQKIWRSFQALGAIAFAYSYSLILIEIQSLTVFDHVDIALSGLDHVESVLAFG

SEQ ID NO: 84 - DNA, 1524 bp, Nicotiana tabacum
  source 1..1524: mol_type = unassigned DNA
atggcagaagaaaaactagccagtaataatggccaacctcagatagttacaagtattcaggattttgatcctccaaagaaaccaaaaagaaacaaatatgcaatagcttgttctttcttagcttctttggcttcaatcttgcttggttatgatattggagtaatgagtggagccataatctacattaagaaagacctccatatcaccgatacccaagtagagattctagtaggaatcctcaatatctactctctctttggttctgccgccgccggccgtacatccgactggattggccgccggtacactatggtggttgctgccgcaatcttctttgccggagctcttctgatgggttttgctaccacatatgcatttctcatggtgggtcgtttcgttgccggagtcggagtcgggtacgcactcatggttgctccagtttacacagccgaagtctcgccggcgtcttctcgcggatttctaacctccttcccggaagttttcattaatggaggtatattgctgggttacgtgtctaacttcgcgttttcaagacttccaactaacttgagttggcgattcatgcttggaatcggagcagtcccatcagtattcttagccgtaagtgtactcgccatgcctgagtcacctcgttggctcgtgatgcagggtcgactcggcgatgcaaggcgagttctgaacaaaacctccgattccttagaagaagctcagttaagactcgccgatattaaagcagccgccggcataccggaacactgcaacgacgacatcgtcgaggtacccaaacgccctaaaggcgacaacgtatggcgcgaattgatcttctcaccaacgccggccgtccgtcacatactactcaccggcgtcggaattcatttctttcagcaagcaagtggaatcgacgccgtcgttttgtacagcccgaggatttttgaaaaagcagggattaaatccgatcacgacaagttgctcgccaccgtagccgtcggattcgtcaaaacaattttcatactcgtagccacattcatgctcgacaaatccggccggcggccattgctgttaaccagtgtcgccggaatggttataacaatggtgttggcttacgtggcacttttctcaatcgggatgggtccaataacgtgggtttacagttcggagatttttccgttgaggctacgggcaactggatgcagtataggtgtggcggtaaacagggtaacaagtggagtagtgtcaatgacgtttttgtccctggtaaagtcgataacaattggcggtgcgttctttttgtacttcggattggcggcggtggcgtttgtgtttttctttacgttgatgccggaaacacaagggaagactctagaagaaatggaggcattgtttggatctttctgggactggagaaaaacggctagagagctgaaagaagccaagataacggagaagaacagcaacgataacggccaaattcagatgggtactgcaccggctattagcagttaa

SEQ ID NO: 85 - protein, 507 aa, Nicotiana tabacum
MAEEKLASNNGQPQIVTSIQDFDPPKKPKRNKYAIACSFLASLASILLGYDIGVMSGAIIYIKKDLHITDTQVEILVGILNIYSLFGSAAAGRTSDWIGRRYTMVVAAAIFFAGALLMGFATTYAFLMVGRFVAGVGVGYALMVAPVYTAEVSPASSRGFLTSFPEVFINGGILLGYVSNFAFSRLPTNLSWRFMLGIGAVPSVFLAVSVLAMPESPRWLVMQGRLGDARRVLNKTSDSLEEAQLRLADIKAAAGIPEHCNDDIVEVPKRPKGDNVWRELIFSPTPAVRHILLTGVGIHFFQQASGIDAVVLYSPRIFEKAGIKSDHDKLLATVAVGFVKTIFILVATFMLDKSGRRPLLLTSVAGMVITMVLAYVALFSIGMGPITWVYSSEIFPLRLRATGCSIGVAVNRVTSGVVSMTFLSLVKSITIGGAFFLYFGLAAVAFVFFFTLMPETQGKTLEEMEALFGSFWDWRKTARELKEAKITEKNSNDNGQIQMGTAPAISS

SEQ ID NO: 86 - DNA, 1191 bp, Nicotiana tabacum
  source 1..1191: mol_type = unassigned DNA
atgtctagcaatggaaaaatagaggaaaataaggttccattattagattatgcatcctctaatatccacaagagttatatattagaacaaaaccaagatgaacaaagttttgcaaatagattttggattgaaacaaagaagctatggcatatagttggccctgctatttttagtagaatagtttcttactccatgtttgtcatcactcaagcttttgctggtcatcttggtgatcttgaacttgctgctatgtccattgctagtaatgttgttcttggcttcgatttcggcctcatgggttacagtactaatatatcagcattttctaaaatatacatacatatgttcatggatattttattgaactttgcgggtactagtgaaccctcgcttggtaacatggtttcgcctccgtttgctactatacaaaagcttccgttatgtcaaaaagattcaacaatttatccaatttgcatagtacgaagcttggagaactggtactacagggtgctaattgtgatgactggtaatttggaaaatgctaaaattgctgtggacgcattgtctatatgcatgaatatcaatggttgggaactaatgattcctcttggattcttcgctggaactggaagacaaaaccatagggccaataaatcaataattgtcaaccagttagtagcagttacacaatcaatagtgattggtttatttttctggattttgatcatatttttccacaatgaacttgctcttattttctcaactagccaacctgttcttgaagctgttcataaactctctatcctcttagccttcactgttctcctcaacagcgttcagcccattctctcgggggttgctgttggatctggatggcaagcatatgttgcatatataaatttgggttgctattatttacttggagtacctcttggtttcataatgggatggggtttccactatggtgtcatgggcatctgggctggaatgatctttggtggaactgcaattcagactttgattttggctataatcacaataagatgtgattggaataaagagcaccaacatcagcaaaagaaagaagaagaagaagaagaagaagaagaagaagaagaagaagaagaaggaggaggaggaagagaagaagaagaaggaggaggaggaggaggaggaggagaaagagactga

SEQ ID NO: 87 - protein, 396 aa, Nicotiana tabacum
MSSNGKIEENKVPLLDYASSNIHKSYILEQNQDEQSFANRFWIETKKLWHIVGPAIFSRIVSYSMFVITQAFAGHLGDLELAAMSIASNVVLGFDFGLMGYSTNISAFSKIYIHMFMDILLNFAGTSEPSLGNMVSPPFATIQKLPLCQKDSTIYPICIVRSLENWYYRVLIVMTGNLENAKIAVDALSICMNINGWELMIPLGFFAGTGRQNHRANKSIIVNQLVAVTQSIVIGLFFWILIIFFHNELALIFSTSQPVLEAVHKLSILLAFTVLLNSVQPILSGVAVGSGWQAYVAYINLGCYYLLGVPLGFIMGWGFHYGVMGIWAGMIFGGTAIQTLILAIITIRCDWNKEHQHQQKKEEEEEEEEEEEEEEEGGGGREEEEGGGGGGGGERD

SEQ ID NO: 88 - DNA, 2223 bp, Nicotiana tabacum
  source 1..2223: mol_type = unassigned DNA
atggattcttctaaaaacaacatttaccagccatttttagataacaatggaaattctagtttgtcacctcagttttctgaaacccataattttgaatcaagcaatgagttagaaactgtacttttagacactgaaataccactatggaataaactccgtttagctacatggattgaaatgaagctcctttttcaccttgctgcacctgctgttatggtttatatgattaattatattatgtctatgtcaactcaaatattttctggtcatttgggtaatcttgagctggctgctgcttcacttggaaatactggaattcaagtttttgcttatggtcttatgttgggaatgggaagtgcagttgaaacactatgtgggcaagcatttggagcaaagaaatatgatatgttaggagtttatctgcaaagatcaacaattctcctaactctaacaggaattttactcgctttcgtgtacattttctgcaagccaattctaatatttctaggcgaatcggaaagaattgcttcagcagctgccttattcgtgtacggtctaattccccaaattttcgcctacgcgttaaatttcccaattcagaagtttctacaagctcagagtattgtagcgccaagtgcatacatttcagctgcgactttagtgttgcatttagtgatgagttgggttgtgatttacaagattgggcttggtctactcggggcgtctttgatactgagtttgtcatggtggataatcgtaataggccaatttgtgtatattgtgaaaagtgagaggtgtaagcaaacatggaatgggttcagtttgcaggcgttttccggcttgccggtgtttttcaagttatcagcagcgtctgcggtgatgttgtgcttggagacatggtactttcaaattgtggttttgctcgctggattgcttgaaaatcctgaattgtctttggatgctctttctatttgcatgacaatttctggttgggtattcatgatatcagttggattcaatgcagcagcaagtgtgagagtgagcaatgaactaggagcaaggcatccaaaatcagcagctttttctgttgttgtggttacgtcgtggtctttcattctctcggtgattgctgcagtcatcgttcttgcattgcgtaatctcatcagttatgccttcactggtggtgaagttgttgctcaagctgtctccgatctttgtccattgctcgcgatttcccttatactcaatggaattcaacccgtcttgtcaggtgtggctgttggctgtggatggcaaacttttgtggcctatgtaaatgttggctgctattacattgtcggagttccattgggtgctctcctcggcttccatttcaaacttggagctaagggtatatggtcaggaatgctgggaggtacggtgatgcagacaatcattttgatatggatcactgctcgaaccgattggaataaagaggccaaggggttggaattcactctcagccacctcatccggttatatcgacctcgactttttcgaggattaatcaagctccaacaccgatcaacgaagtccttttttgcaagcattgatgaggacaaagaccgaggttggatgagccggttcgtgcgggtaaggacctgcgacctcatccctgaggagaaactgctgttccccgagaagtggaaccctgatccggttgcatggatgcctcacgtggtccccgacctcgaagactggttcgaaagctggctgcaacttcctcccatgccgagcgctgctggcgtgacatggcaaggggcagataggaggctaagaatcatagtgagctcatcttgcctcggggatgttgctgaaataaggcctacctcgcctggggaagaggtggatcccaaaccggttaaggacaagaaaaggagaagggcctcaccctctgataccccaaagtccaagaagattaaggcccgtaaattgaaggacgattctgccactctatctgccgacgtagtccaacagctacgagatgaagaagaggagggggaagatgctggttgtgatctggtgcctcgaaagagggggagcgtcgaagctccaaaaatagttgggccggtgatggtcgaggagactcatccttggaccgaggagatctcggaaggtactccgagcagagttcccgagtcatcgggggcccaagatattcccgtcgtgatgaaaaatcggtga

SEQ ID NO: 89 - protein, 740 aa, Nicotiana tabacum
MDSSKNNIYQPFLDNNGNSSLSPQFSETHNFESSNELETVLLDTEIPLWNKLRLATWIEMKLLFHLAAPAVMVYMINYIMSMSTQIFSGHLGNLELAAASLGNTGIQVFAYGLMLGMGSAVETLCGQAFGAKKYDMLGVYLQRSTILLTLTGILLAFVYIFCKPILIFLGESERIASAAALFVYGLIPQIFAYALNFPIQKFLQAQSIVAPSAYISAATLVLHLVMSWVVIYKIGLGLLGASLILSLSWWIIVIGQFVYIVKSERCKQTWNGFSLQAFSGLPVFFKLSAASAVMLCLETWYFQIVVLLAGLLENPELSLDALSICMTISGWVFMISVGFNAAASVRVSNELGARHPKSAAFSVVVVTSWSFILSVIAAVIVLALRNLISYAFTGGEVVAQAVSDLCPLLAISLILNGIQPVLSGVAVGCGWQTFVAYVNVGCYYIVGVPLGALLGFHFKLGAKGIWSGMLGGTVMQTIILIWITARTDWNKEAKGLEFTLSHLIRLYRPRLFRGLIKLQHRSTKSFFASIDEDKDRGWMSRFVRVRTCDLIPEEKLLFPEKWNPDPVAWMPHVVPDLEDWFESWLQLPPMPSAAGVTWQGADRRLRIIVSSSCLGDVAEIRPTSPGEEVDPKPVKDKKRRRASPSDTPKSKKIKARKLKDDSATLSADVVQQLRDEEEEGEDAGCDLVPRKRGSVEAPKIVGPVMVEETHPWTEEISEGTPSRVPESSGAQDIPVVMKNR

SEQ ID NO: 90 - DNA, 1404 bp, Nicotiana tabacum
  source 1..1404: mol_type = unassigned DNA
atgcttgagcgagcctctgttatagaattgaaaaatctattccggctagcagccccatcagttattgtttatttgctcaacaatatcacttcaatgtctacccaactattctgtggtcatctcggtaatcttgaactcgctgctgcttcccttggcaatgaaggtattcaactcttggcctatggcgtcatgttagggatggggagtgcagtggagacactatgtgggcaggcatatggagctcacaagtatggaagtcttggaatatatcttcaaagatcaactatcctcttgatgttatccggaataccgataatggtggcttacttattctcaaagccaattttaatttttttaggagaatcagagaaagttgcatcagcagctgcactatttgtttatggtttaattcctcaaatatttgcttatgctgccaattttcctatccaaaagttcttgcaagcccaaagtattgtaaatcccagcgcatatatagcagcagcgacattagtcctccatcttttcctaacatggattgtgctttatgtatttgagtggggattgtttggaggagccttggttctaagtatttcgtggtggatagtagtcattgcacaatttatgtacatattgtggagtgatacatgcaagaaaacatggagtggatttagtctgcaagcgttttctgggctgtgggatttctttaagttgtcagctgcttcatctgtgatgttgtgtttggaggcgtggtactttcagattttggttttagttgctggtttgctaccaaatcctgaagtggcattggattctctagctgtttgtaacacaattcttggatgtgtgttcatgttatctgttggtttcaacgctgctgcaagtgtaagggtgagcaatgaattgggagctggacatccaaaatcagctgcattttcagttgtggtggtaacattaagctcgttcgtgattgctgtagtcattgccatagtagtgatgttgcttcgcgatgtgatgagttatgcattcacaggcggggaaaccattgctaaagcatcctcagaacttgcaccactcttggctgcctcagtagttctcaatggtattcaacctgttttatctggggtggccgttgggtgtggatggcaaggttttgtagcttacgttaacgtcggatgttactacgttgttggcattccattgggtgttcttcttggtttcaaattcaaactcggagctaaggggattatggctgggcatgtttggaggaacagccatacaaactctaatcttactatgggccacttttcgaaccaattgggacgacgaggtcaggtggagaaaacgaaaaatcgattaaataagtggcgagatagcagtaaaaccccattgttgaacgaatcatga

SEQ ID NO: 91 - protein, 467 aa, Nicotiana tabacum
MLERASVIELKNLFRLAAPSVIVYLLNNITSMSTQLFCGHLGNLELAAASLGNEGIQLLAYGVMLGMGSAVETLCGQAYGAHKYGSLGIYLQRSTILLMLSGIPIMVAYLFSKPILIFLGESEKVASAAALFVYGLIPQIFAYAANFPIQKFLQAQSIVNPSAYIAAATLVLHLFLTWIVLYVFEWGLFGGALVLSISWWIVVIAQFMYILWSDTCKKTWSGFSLQAFSGLWDFFKLSAASSVMLCLEAWYFQILVLVAGLLPNPEVALDSLAVCNTILGCVFMLSVGFNAAASVRVSNELGAGHPKSAAFSVVVVTLSSFVIAVVIAIVVMLLRDVMSYAFTGGETIAKASSELAPLLAASVVLNGIQPVLSGVAVGCGWQGFVAYVNVGCYYVVGIPLGVLLGFKFKLGAKGIMAGHVWRNSHTNSNLTMGHFSNQLGRRGQVEKTKNRLNKWRDSSKTPLLNES

SEQ ID NO: 92 - protein, 353 aa, Nicotiana tabacum
MEHPGLSTNMRRILLLISCLILAVGICGGPLMMRLYYVEGGSRIWLSSWLQTGGWPLTLIPLAILYYYRRKTEGSNAKFYLMTPRIFIASFVIGVATGLDDFLYSWGGSKLPVSTSSLLLAAQLAFTAVGAFFIVKLKLSPFSINAVVLLTVGAVLLGIRSNGDRPEGVTSKEYIIGFMMTLLAAALYGVILPCIELIYMKAKQAITSTLVLEIQMIMSFAATAFCTVGMIANKDFQAMSREAKQFNVGEARYYTVIVCTAAIWQCFFVGIIGVIYCSSSLMSGVMIAVLLPVTEVLAVIFFKENFSGEKGLALFLSLWGFVSYFYGEFRQTKKQKNTSPEAEMTITTHTESV

SEQ ID NO: 93 - protein, 352 aa, Nicotiana tabacum
MEHPGLSTTMRRILLLISCLILAVGICGGPLMMRLYYVEGGSRIWLSSWLQTGGWPLTFIPLAFLYYYRRKIEGSNAKFYLMTPRIFIAAFVIGIATGLDDFLYSWGGSKLPVSTSSLLLAAQLAFTAVGAFFIVKLKLSPFSINAVVLLTVGAVLLGIRSNGDRPEGVTSKEYIIGFMMTLLAAALYGVILPCIELIYMKAKQAITATLVLEIQMIMSFAATAFCTVGMIANKDFQAMSREAKQFNLGEARYYTVIVCTAAIWECFFVGIIGVIYCSSSLMSGVMIAVLLPVTEVLAVIFFKEKFSGEKGLALFLSLWGFVSYFYGEFRQTKKEKNKSPEAEMTTTHTESV

SEQ ID NO: 94 - protein, 1263 aa, Solanum lycopersicum
MEDNNNNGEKKRDEDQKVSFYKLFSFADKFDIALMIIGTIGAIGNGLTQPLMTLIFGQLVNSFGSSNSDEVVHEISKVSIYYVYLAIGAGVASLLQMSCWMVTGERQATRIRGLYLKTILRQDIAFFDTETTTGEVIGRMSGDTILIQDALGEKVGKFIQFISTFVGGFIVAFFKGWLLSIVLVSCIPALVIAGGAMALIMSKMSSRGQVAYAQAGNVVEQTIGAIRTVSAFTGEKLAIDKYDSKLKIACASTVQQGLVSGIGLGTVLLIVFSTYGLAVWYGSKLIIERGYNGGDVINVIMAIMTGGMSLGQTTPSLNAFAAGQAAAYKMFETINRKPLIDTSDTSGVVLENIKGEIELKDVYFKYPARPDVQIFSGFSLVVPSGKTVALVGQSGSGKSTVISLLERFYDPEAGEVLIDGVNLKKFQLKWLRQQMGLVSQEPILFATTIKENISYGKENATEDEIKTAIELANAAKFLDKLPQGLDTMVGEHGTQLSGGQKQRLAIARAILKNPRILLLDEATSALDAESERIVQEALEKVMANRTTVVVAHRLTTIRNADLIAVVNAGKLLEKGTHTELIQDPNGAYSQLVRMQGGNREEENMKNIDLEKVDLTTDFDNNLSRSSSQRLSAMRRSTSQGSSRHSFTLNYTVPGLIGIHEAEIGNENKGKEDKGSSKKRKKVSIRRLAGLNKPELPYLLLGSLAAIIHGLIFPLFGLLLSTAIKIFFYPPQKLRIESRFWALMYFGLGVVTLLVVPFQNYLFGVAGGKLIERIRSLTFKKVVHQEISWFDDPAHSSGAIGARLSTDASTVRTLMGDALALIVQNIATVVAGLVIAFTANWILALIILLVMPLIGVQGFLQTKMYKGFSADAKVMYEEASQIANDAVGSIRTVASFCAEEKVMDMYQKKCEGPMKQGVKIGIVSGASLGFGSFILYCTNAFCFYIGSVLIQHGLASFGQVFKVFFALTLSAVGVTQSTGMAPDANKAKDSIASIFDILDRKPEIDSSSDVGTTLAAVRGDIEFKHVSYRYATRPDVQIFKDLCLTIPSGKTVALVGESGSGKSTVISLIERFYNPESGSIYLDGVEIRQFKISWLRQQMGLVSQEPVLFNETIRDNIAYSRQGHATEEEIIEAAKSANAHNFISSLPQGYDTSVGERGIQLSGGQKQRIAIARAILKDPKILLLDEATSALDAESERIVQEALDRVMVNRTTVVVAHRLTTIKGADVIAVVKNGVIAEEGRHDALMNIKDGVYASLVALHMTSA

SEQ ID NO: 95 - protein, 500 aa, Nicotiana tabacum
MGKSMKSEVEQPLLIAAHGGSSELEEVLSDTQLPYFRRLRYASWIEFQLLYRLAAPSVAVYMINNAMSMSTRIFSGQLGNLQLAAASLGNQGIQLFAYGLMLGMGSAVETLCGQAYGAHRYEMLGVYLQRATVVLSVTGIPLTVVYLFSKNILLALGESKLVASAAAVFVYGLIPQIFAYAVNFPIQKFLQAQSIVAPSAFISLGTLFVHILLSWVVVYKIGLGLLGASLVLSFSWWIIVVAQFIYIIKSERCKATWAGFRWEAFSGLCQFVKLSAGSAVMLCLETWYMQILVLLSGLLKNPEIALASISVCLAVNGLMFMVAVGFNAAASVRVSNELGAAHSKSAAFSVFMVTFISFLIAVVEAIIVLSLRNVISYAFTEGEIVAKEVSELCPFLAVTLILNGIQPVLSGVAVGCGWQAFVAYVNVGCYYGVGIPLGCLLGFKFDLGAKGIWTGMIGGTVMQTVILLWVTFRTDWNKKVECAKKRLDKWENLKGPLNKE

SEQ ID NO: 96 - protein, 1227 aa, Solanum lycopersicum
MSSSTEKAFENKNKNSIGIIFRYADGKDILLMFLGTIGAIGDGISTNCLLVYVSQLFNSLGYGKTQQNDHNFMEQIEKCSLYFVLLGLGVMVVAFMEGYCWSKTSERQVLKIRYKYLEAILRQEVGFFDSQEATTSEITNGISKDTSLIQEVLSEKVPLFVMHTTVFISGVVFSAYFSWRLAIVALPTIFLLIIPGLIYGKYLLYLSGKSFKEYSKANGIVEQALSSIKTIYSFTAEKSVIERYSLILDGTIKLGMKQGIAKGLAVGSTGLSFAIWALLAWYGSHLIMHNGESGGRIYAAGVSFVLGGLSLGMALPEVKYFTEASVAASRIFDRIDRVPEIDGEDTRGLVLEDIRGEVEFRNVKFTYPSRPDTVVLKDFNLKIEAGKTVALVGSSGSGKSTAIALIQRFYDASAGAICIDSVEIKSLQLKWLRGKMGLVSQENALFGTSIKENIMFGKVDATMDEVVAAAMTANAHNFITQLPEGYETKIGERGALLSGGQKQRIAIARAIIKNPVILLLDEATSALDSESETLVQNALDQAIVGRTTLVVAHKLSTVRNADLIAVVSNGCISELGAHYELMEKDGQYARLAKFQRQFSSIDQEQSAEPRISSVARSSAGMRASPAVSASPLRIEDSPIQASPHPPPSFTRLLSLNLPEWKQGIIGILSAIAFGSVQPVYALTIGGMISAFYSPSHEEMQSRIQKYCMIFIILCLVSVVLNLCQHYNFAYMGERLTRRIRLQMLEKILSFEAAWFDEEQNSSGALCCRLSNEAAMVKSLVADRVSLLVQSTSAVTVAMVMGLIVAWKLALVMIVVQPLTILCFYTRKVLLSTMTAKFVKAQCRSTQIAVEAVYNHRIVTSFGSIDKVLDIFDEAQDEPRKEARKKSWLAGIGIGSAQGLTFICWALDFWYGGKLVNAGEISAADVFKTFFILVSTGKVIAEAGSMTSDLAKGSTVVASIFSILDRKSLIEGSNEAKNNSMGTKMTGRIEMKKVDFAYPSRPDRLVLHEFSLEVKAGTSIGLVGKSGCGKSTVIALIQRFYDADKGSLKIDGMDIRLLDLGWYRRNMALVSQEPVIYSGSIRENILFGKLNASENEVVEAAKAANAHEFISSLKNGYETECGDRGVTISGGQKQRIAIARAIIRNPSILLLDEATSALDVQSEQLVQEALDQLMVGRTTVVVAHRLNTIRNLDSIAFISEGKVLEKGTYSYLKDKRGAFFNLVNLQST

SEQ ID NO: 97 - protein, 276 aa, Solanum lycopersicum
MTSVSHTHPLVYTFGILGNLVSFMVFIAPVPTFYRIVKKKSSEGFHSLPYVVGLFSAMLWIYYAMVKTNVTLLITINSFGCIAETIYVAIYFTYATKKARMKTLGLVLLLNFGVFGLILFLTQILCQGTKRAEVIGWICMAFSISVFVAPLSIMGRVIRTKSVEFMPFNLSLALTVSAVMWFLYGLLLKDVYVAVPNIPGMILGVLQMILYGIYRNSKSNNVATEKELPIVVKVDQEQPTKVNSEVYPVNISSLDSENGEAKDGKNLQDPQMNSQV

(19) United States
(12) Patent Application Publication
(10) Pub. No.: US 2025/0064011 A1
(43) Pub. Date: Feb. 27, 2025

(54) TOBACCO PLANTS HAVING ALTERED AMOUNTS OF ONE OR MORE ALKALOIDS IN LEAF AND METHODS OF USING SUCH PLANTS

(71) Applicant: Altria Client Services LLC, Richmond, VA (US)

(72) Inventors: Jesse Frederick, Richmond, VA (US); Yanxin Shen, Glen Allen, VA (US); Dongmei Xu, Glen Allen, VA (US); Ujwala Warek, Chester, VA (US); James Arthur Strickland, Richmond, VA (US)

(73) Assignee: Altria Client Services LLC, Richmond, VA (US)

(21) Appl. No.: 18/943,128

(22) Filed: Nov. 11, 2024

Related U.S. Application Data
(63) Continuation of application No. 17/892,461, filed on Aug. 22, 2022.
Application No. 17/892,461 is a continuation of application No. 17/005,942, filed on Aug. 28, 2020, now Pat. No. 11,452,275.
Application No. 17/005,942 is a continuation of application No. 15/914,033, filed on Mar. 7, 2018, now Pat. No. 10,813,318.
Application No. 15/914,033 is a division of application No. 14/563,211, filed on Dec. 8, 2014, now abandoned.
(60) Provisional application No. 62/011,304, filed on Jun. 12, 2014.
(60) Provisional application No. 61/912,752, filed on Dec. 6, 2013.

(51) Int. Cl. A01H 6/82; A01H 1/02; A01H 1/06; A01H 5/12; A24B 15/20; C07K 14/415; C12N 15/82; C12Q 1/6895; G01N 33/00

(52) U.S. Cl. CPC A01H 6/823; A01H 1/02; A01H 1/06; A01H 5/12; A24B 15/20; C07K 14/415; C12N 15/8243; C12Q 1/6895; G01N 33/0098; C12Q 2600/13; C12Q 2600/156; C12Q 2600/16

(57) ABSTRACT

This disclosure provides a number of sequences involved in the transport of alkaloids from the root to the leaf in tobacco, methods of using such sequences, tobacco plants carrying modifications to such sequences, and tobacco products made from such plants.

### CROSS-REFERENCE TO RELATED APPLICATIONS

This application is a continuation of U.S. patent application Ser. No. 17/892,461, filed Aug. 22, 2022, which is a continuation of U.S. patent application Ser. No. 17/005,942, filed Aug. 28, 2020 (now U.S. Pat. No. 11,452,275), which is a continuation of U.S. patent application Ser. No. 15/914,033, filed Mar. 7, 2018 (now U.S. Pat. No. 10,813,318), which is a divisional of U.S. patent application Ser. No. 14/563,211, filed Dec. 8, 2014, which claims the benefit of U.S. Provisional Application No. 62/011,304, filed Jun. 12, 2014, and U.S. Provisional Application No. 61/912,752, filed Dec. 6, 2013, all of which are incorporated by reference in their entireties herein.

### INCORPORATION OF SEQUENCE LISTING

A sequence listing contained in the file named “P34632US06_SL.XML” which is 204,800 bytes (measured in MS-Windows®) and produced on May 23, 2024, is filed electronically herewith and incorporated by reference in its entirety.

### TECHNICAL FIELD

This disclosure generally relates to tobacco plants.

### BACKGROUND

Attempts have been made to produce low alkaloid varieties of tobacco. However, most such varieties result in low quality leaf and there are no commercial tobacco lines available with reduced leaf alkaloid content. Accordingly, there is a need to identify tobacco genes whose expression can be modulated such that the alkaloid profile in tobacco leaf can be altered, in particular, the profile of leaf nicotinic alkaloids.

### SUMMARY

A number of sequences that are involved in the transport of alkaloids from the root to the leaf in tobacco are described. Methods of using such sequences also are described.

In one aspect, a tobacco hybrid, variety, line, or cultivar is provided. Such a tobacco hybrid, variety, line, or cultivar includes plants having a mutation in one or more endogenous nucleic acids such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, tobacco leaf from the plants exhibits a reduced amount of at least one alkaloid relative to tobacco leaf from a plant lacking the mutation. In some embodiments, cured leaf from the plants exhibits a reduced amount of at least one tobacco specific nitrosamine (TSNA) relative to cured leaf from a plant lacking the mutation. Seed produced by such a tobacco hybrid, variety, line, or cultivar also is provided, where the seed includes the mutation in one or more endogenous nucleic acids having a sequence such as SEQ ID NOS: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90.

In another aspect, a method of making a tobacco plant is provided. Such a method typically includes the steps of inducing mutagenesis in Nicotiana tabacum cells to produce mutagenized cells, obtaining one or more plants from the mutagenized cells, and identifying at least one of the plants that contains a mutation in one or more endogenous nucleic acids such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. Such a method can further include identifying at least one of the plants that contains leaf exhibiting a reduced amount of at least one alkaloid relative to leaf from a plant lacking the mutation. Such a method can also include identifying at least one of the plants where the resulting cured leaf exhibits a reduced amount of at least one TSNA relative to cured leaf from a plant lacking the mutation.

Mutagenesis can be induced using a chemical mutagen or ionizing radiation. Representative chemical mutagens include, without limitation, nitrous acid, sodium azide, acridine orange, ethidium bromide, and ethyl methane sulfonate (EMS). Representative ionizing radiation includes, without limitation, x-rays, gamma rays, fast neutron irradiation, and UV irradiation. Mutagenesis can be induced using TALEN or zinc-finger technology.

In another aspect, a method of producing a tobacco plant is provided. Such a method also can include the steps of crossing at least one plant of a first tobacco line with at least one plant of a second tobacco line and selecting for progeny tobacco plants that have the mutation. Generally, the plant of the first tobacco line has a mutation in one or more endogenous nucleic acids having a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. Such a method further can include selecting for progeny tobacco plants that have leaf exhibiting a reduced amount of at least one alkaloid relative to leaf from a plant lacking the mutation. Such a method also can include selecting for progeny tobacco plants where the cured leaf exhibits a reduced amount of at least one TSNA relative to cured leaf from a plant lacking the mutation.

In another aspect, a tobacco product is provided. Such a tobacco product typically includes cured leaf from a tobacco plant having a mutation in one or more endogenous nucleic acids having a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, the cured leaf contained within the tobacco product exhibits a reduced amount of at least one alkaloid relative to cured leaf contained within a tobacco product that is from a plant lacking the mutation. In some embodiments, the cured leaf within the tobacco product exhibits a reduced amount of at least one TSNA relative to cured leaf contained within a tobacco product that is from a plant lacking the mutation.

In another aspect, a method of producing a tobacco product is provided. Such a method generally includes providing cured leaf from a tobacco plant having a mutation in one or more endogenous nucleic acids having a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, and manufacturing a tobacco product using the cured leaves. In some embodiments, the cured leaf exhibits a reduced amount of at least one alkaloid relative to cured leaf from a plant lacking the mutation. In some embodiments, the cured leaf exhibits a reduced amount of at least one TSNA relative to cured leaf from a plant lacking the mutation.

A mutation as described herein can be, without limitation, a point mutation, an insertion, a deletion, or a substitution.

In still another aspect, a transgenic tobacco plant is provided that includes a plant expression vector. Typically, the expression vector includes a nucleic acid molecule that is at least 25 nucleotides in length and has at least 91% sequence identity to a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, expression of the nucleic acid molecule results in leaf exhibiting a reduced amount of at least one alkaloid relative to leaf from a tobacco plant not expressing the nucleic acid molecule. In some embodiments, expression of the nucleic acid molecule results in cured leaf exhibiting a reduced amount of at least one TSNA relative to cured leaf from a tobacco plant not expressing the nucleic acid molecule. Seed produced by such a transgenic tobacco plant also is provided, where the seed includes the expression vector.

In one aspect, a transgenic tobacco plant is provided that includes a heterologous nucleic acid molecule of at least 25 nucleotides in length, wherein the nucleic acid molecule hybridizes under stringent conditions to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, expression of the heterologous nucleic acid molecule results in leaf exhibiting a reduced amount of at least one alkaloid relative to leaf from a tobacco plant not expressing the nucleic acid molecule. In some embodiments, expression of the heterologous nucleic acid molecule results in cured leaf exhibiting a reduced amount of at least one TSNA relative to cured leaf from a tobacco plant not expressing the nucleic acid molecule. Seed produced by such a transgenic tobacco plant also is provided, where the seed includes the heterologous nucleic acid molecule.

In one aspect, a leaf from a transgenic tobacco plant is provided that includes a vector. Generally, the vector includes a nucleic acid molecule having at least 91% sequence identity to 25 or more contiguous nucleotides of a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, expression of the nucleic acid molecule results in the leaf exhibiting a reduced amount of at least one alkaloid relative to leaf from a tobacco plant not expressing the nucleic acid molecule. In some embodiments, expression of the nucleic acid molecule results in cured leaf exhibiting a reduced amount of at least one TSNA relative to cured leaf from a tobacco plant not expressing the nucleic acid molecule.

In another aspect, a method of making a transgenic plant is provided. Such a method typically includes expressing a transgene in the plant. The transgene encodes a double-stranded RNA molecule that inhibits expression from a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. The double-stranded RNA molecule includes at least 25 consecutive nucleotides having 91% or greater sequence identity to a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, expression of the transgene results in leaf from the plant exhibiting a reduced amount of at least one alkaloid relative to leaf from a plant not expressing the transgene. In some embodiments, expression of the transgene results in cured leaf exhibiting a reduced amount of at least one TSNA relative to cured leaf from a plant not expressing the nucleic acid molecule. In some embodiments, the double-stranded RNA molecule has a sequence such as SEQ ID NOs: 51-56.

In another aspect, a method of altering leaf constituents in a tobacco plant is provided. Such a method generally includes the steps of introducing a heterologous nucleic acid molecule operably linked to a promoter into tobacco cells to produce transgenic tobacco cells, and regenerating transgenic tobacco plants from the transgenic tobacco cells. Typically, the heterologous nucleic acid molecule includes at least 25 nucleotides in length and has at least 91% sequence identity to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. Such transgenic tobacco plants exhibit altered leaf constituents. Such a method further can include selecting at least one of the transgenic tobacco plants having leaf that exhibits a reduced amount of at least one alkaloid relative to leaf from a tobacco plant not expressing the heterologous nucleic acid molecule. Such a method further can include selecting at least one of the transgenic tobacco plants having cured leaf exhibiting a reduced amount of at least one TSNA relative to cured leaf from a tobacco plant not expressing the heterologous nucleic acid molecule.

In another aspect, a cured tobacco leaf from a transgenic tobacco plant is provided that includes a vector. Generally, the vector includes a nucleic acid molecule having at least 91% sequence identity (e.g., at least 92, 93, 94, 95, 96, 97, 98, 99 or 100% sequence identity) to 25 or more contiguous nucleotides of a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90. In some embodiments, expression of the nucleic acid molecule results in leaf exhibiting a reduced amount of at least one alkaloid relative to leaf from a tobacco plant not expressing the nucleic acid molecule. In some embodiments, expression of the nucleic acid molecule results in cured leaf exhibiting a reduced amount of at least one TSNA relative to cured leaf from a tobacco plant not expressing the nucleic acid molecule. In some embodiments, the nucleic acid is in sense orientation, while, in some embodiments, the nucleic acid is in antisense orientation.

In still another aspect, a transgenic tobacco plant is provided that includes a plant expression vector. Generally, the expression vector includes a nucleic acid molecule having at least 95% sequence identity to a sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, or a fragment of any of those sequences encoding a functional polypeptide. In some embodiments, expression of the nucleic acid molecule or a functional fragment thereof results in leaf exhibiting an increased amount of at least one alkaloid relative to leaf from a tobacco plant not expressing the nucleic acid molecule or functional fragment thereof. Seed produced by such a transgenic tobacco plant also is provided, where the seed includes the expression vector.

In another aspect, a transgenic tobacco plant is provided that includes a heterologous nucleic acid molecule. Generally, the nucleic acid molecule hybridizes under stringent conditions to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, or a fragment thereof encoding a functional polypeptide. In some embodiments, expression of the heterologous nucleic acid molecule or functional fragment thereof results in leaf exhibiting an increased amount of at least one alkaloid relative to leaf from a tobacco plant not expressing the nucleic acid molecule or functional fragment thereof. Seed produced by such a transgenic tobacco plant also is provided, where the seed includes the heterologous nucleic acid molecule.

In one aspect, a leaf from a transgenic tobacco plant is provided that includes a vector. Typically, such a vector includes a nucleic acid molecule having at least 95% sequence identity to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, or a fragment thereof encoding a functional polypeptide. In some embodiments, expression of the nucleic acid molecule or functional fragment thereof results in the leaf exhibiting an increased amount of at least one alkaloid relative to leaf from a tobacco plant not expressing the nucleic acid molecule or functional fragment thereof.

In another aspect, a method of altering leaf constituents in a tobacco plant is provided. Such a method typically includes the steps of introducing a heterologous nucleic acid molecule operably linked to a promoter into tobacco cells to produce transgenic tobacco cells, and regenerating transgenic tobacco plants from the transgenic tobacco cells, wherein the transgenic tobacco plants have altered leaf constituents. The heterologous nucleic acid molecule typically has at least 95% sequence identity to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, or a fragment thereof encoding a functional polypeptide. Such a method further can include selecting at least one of the transgenic tobacco plants having leaf that exhibits an increased amount of at least one alkaloid relative to leaf from a tobacco plant not expressing the heterologous nucleic acid molecule or functional fragment thereof. In some embodiments, the heterologous nucleic acid molecule is introduced into the tobacco cells using particle bombardment, Agrobacterium-mediated transformation, microinjection, polyethylene glycol-mediated transformation, liposome-mediated DNA uptake, or electroporation.

In one aspect, a cured tobacco leaf from a transgenic tobacco plant is provided that includes a vector. Typically, the vector includes a nucleic acid molecule having at least 95% sequence identity to a nucleic acid sequence such as SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, or a fragment thereof encoding a functional polypeptide. In some embodiments, expression of the nucleic acid molecule or functional fragment thereof results in tobacco leaf exhibiting an increased amount of at least one alkaloid relative to leaf from a tobacco plant not expressing the nucleic acid molecule or functional fragment thereof.

Representative alkaloids include, without limitation, nicotine, nornicotine, anabasine, myosmine, and anatabine. Typically, the amount of one or more alkaloids is determined using high performance liquid chromatography (HPLC)—mass spectroscopy (MS) or high performance thin layer chromatography (HPTLC).

Suitable tobacco plants for use in the methods described herein can be a Burley type, a dark type, a flue-cured type, a Maryland type, or an Oriental type. Suitable tobacco plants for use in the methods described herein typically are from N. tabacum, and can be from any number of N. tabacum varieties. A variety can be BU 64, CC 101, CC 200, CC 13, CC 27, CC 33, CC 35, CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpao tobacco, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY907LC, KTY14×L8 LC, Little Crittenden, McNair 373, McNair 944, msKY 14×L8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN90LC, TN 97, TN97LC, TN D94, TN D950, TR (Tom Rosson) Madole, VA 309, or VA359.

In another aspect, a method of screening plants is provided. Such a method typically includes providing plant material from a mutant plant as described herein, and determining the amount of one or more alkaloids in plant tissue. In some embodiments, the plant tissue is leaf. In some embodiments, the plant tissue is root. Such a method can further include determining the amount of one or more TSNAs in plant tissue.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is an alignment of the novel Nup sequences described herein with previously known N. tabacum Nup1 and Nup2. C32288 (SEQ ID NO:2); C40974 (SEQ ID NO:4); C42033 (SEQ ID NO:6); Nup1 (SEQ ID NO:92); Nup2 (SEQ ID NO:93). Nup1-predicted transmembrane helices are denoted by boxes.

FIG. 2 is an alignment of the four novel MDR sequences described herein (DC3222 (SEQ ID NO:10); C11099 (SEQ ID NO:16): DC62783 (SEQ ID NO:12); DC26451 (SEQ ID NO:14)) with a representative MDR polypeptide (Accession #XP_004233862; SEQ ID NO:94). The boxed regions denote the conserved ATPase domain.

FIG. 3 is an alignment of the novel MATE sequences (C9954 (SEQ ID NO:26); C39106 (SEQ ID NO:28); C46276 (SEQ ID NO:22); C48594 (SEQ ID NO:24); DC38072 (SEQ ID NO:20); DC58421 (SEQ ID NO:18)) with N. tabacum MATE1. The boxes indicate predicted transmembrane domains; the predicted N-terminal localization is shaded, with the predicted conserved cleavage site shown with the arrow.

FIG. 4A is an alignment of one of the novel MDR sequences, C11099 (SEQ ID NO:16), with a putative ABC transporter B family member 8-like from Solanum lycopersicum (ABCB-8 (SEQ ID NO:96)), and FIG. 4B is an alignment of one of the other transporter sequences identified, C43677 (SEQ ID NO:40), with a bidirectional sugar transporter SWEET12-like from Solanum lycopersicum (SEQ ID NO:97).

FIG. 5 is a graph showing the amount of nicotine in leaves and roots of N. alata and N. tabacum.

FIG. 6 is a schematic of the construct used to express RNAi molecules in transgenic plants as described herein.

FIG. 7 is a schematic of the construct used for TALEN mutagenesis.

FIG. 8A is the leaf to root ratio of nicotine content after nicotine feeding using the float tray protocol and FIG. 8B is the root and leaf nicotine content of individual plants.

FIG. 9A is the nicotine content of leaves of individual plants after nicotine feeding using the bottomless pot feeding protocol and FIG. 9B is the nicotine content of leaves before and after feeding of the same plants.

FIG. 10 describes regions of a transformation cassette sequence (SEQ ID NO:57).

### DETAILED DESCRIPTION

Previous attempts to modify the pathway to produce low alkaloid varieties of tobacco sometimes resulted in low quality leaf. Currently, there are no commercial tobacco lines with reduced leaf alkaloid content that provide the same quality of cured leaf as those containing standard alkaloid content (e.g., from wild type tobacco plants).

This disclosure is based on the discovery of novel nucleic acids encoding alkaloid transporter and regulatory polypeptides from N. tabacum. Such nucleic acids, SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90, and the polypeptides encoded thereby, SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91, are described and characterized herein. Based on this discovery, the level of expression of such nucleic acid sequences and/or the function of such polypeptides can be modulated in N. tabacum and the resulting effect on alkaloid transport in plants can be evaluated. Modulating polypeptide function and/or genes expression can permit improved control of the alkaloid composition in tobacco leaf and resulting tobacco products.

### Nucleic Acids and Polypeptides

Novel nucleic acids are provided herein (see, for example, SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90). As used herein, nucleic acids can include DNA and RNA, and includes nucleic acids that contain one or more nucleotide analogs or backbone modifications. A nucleic acid can be single stranded or double stranded, which usually depends upon its intended use. The novel nucleic acids provided herein encode novel polypeptides (see, for example, SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91).

Also provided are nucleic acids and polypeptides that differ from SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90 and SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91, respectively. Nucleic acids and polypeptides that differ in sequence from SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90 and SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91, can have at least 50% sequence identity (e.g., at least 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% sequence identity) to SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90 and SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91, respectively.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the computer program ClustalW and default parameters, which allows alignments of nucleic acid or polypeptide sequences to be carried out across their entire length (global alignment). Chenna et al., 2003, Nucleic Acids Res., 31(13):3497-500. ClustalW calculates the best match between a query and one or more subject sequences, and aligns them so that identities, similarities and differences can be determined. Gaps of one or more residues can be inserted into a query sequence, a subject sequence, or both, to maximize sequence alignments. For fast pairwise alignment of nucleic acid sequences, the default parameters can be used (i.e., word size: 2; window size: 4; scoring method: percentage; number of top diagonals: 4; and gap penalty: 5); for an alignment of multiple nucleic acid sequences, the following parameters can be used: gap opening penalty: 10.0; gap extension penalty: 5.0; and weight transitions: yes. For fast pairwise alignment of polypeptide sequences, the following parameters can be used: word size: 1; window size: 5; scoring method: percentage; number of top diagonals: 5; and gap penalty: 3. For multiple alignment of polypeptide sequences, the following parameters can be used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; and residue-specific gap penalties: on. ClustalW can be run, for example, at the Baylor College of Medicine Search Launcher website or at the European Bioinformatics Institute website on the World Wide Web.

Changes can be introduced into a nucleic acid molecule (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90), thereby leading to changes in the amino acid sequence of the encoded polypeptide (e.g., SEQ ID NOs: 2, 4, 6, 8, 10, 12, 14, 16, 18, 20, 22, 24, 26, 28, 30, 34, 36, 38, 40, 42, 44, 46, 48, 50, 71, 73, 75, 77, 79, 81, 83, 85, 87, 89, or 91). For example, changes can be introduced into nucleic acid coding sequences using mutagenesis (e.g., site-directed mutagenesis, PCR-mediated mutagenesis) or by chemically synthesizing a nucleic acid molecule having such changes. Such nucleic acid changes can lead to conservative and/or non-conservative amino acid substitutions at one or more amino acid residues. A “conservative amino acid substitution” is one in which one amino acid residue is replaced with a different amino acid residue having a similar side chain (see, for example, Dayhoff et al. (1978, in Atlas of Protein Sequence and Structure, 5(Suppl. 3):345-352), which provides frequency tables for amino acid substitutions), and a non-conservative substitution is one in which an amino acid residue is replaced with an amino acid residue that does not have a similar side chain.

As used herein, an “isolated” nucleic acid molecule is a nucleic acid molecule that is free of sequences that naturally flank one or both ends of the nucleic acid in the genome of the organism from which the isolated nucleic acid molecule is derived (e.g., a cDNA or genomic DNA fragment produced by PCR or restriction endonuclease digestion). Such an isolated nucleic acid molecule is generally introduced into a vector (e.g., a cloning vector, or an expression vector) for convenience of manipulation or to generate a fusion nucleic acid molecule, discussed in more detail below. In addition, an isolated nucleic acid molecule can include an engineered nucleic acid molecule such as a recombinant or a synthetic nucleic acid molecule.

As used herein, a “purified” polypeptide is a polypeptide that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is “purified.”

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides.

Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

A vector containing a nucleic acid (e.g., a nucleic acid that encodes a polypeptide) also is provided. Vectors, including expression vectors, are commercially available or can be produced by recombinant DNA techniques routine in the art. A vector containing a nucleic acid can have expression elements operably linked to such a nucleic acid, and further can include sequences such as those encoding a selectable marker (e.g., an antibiotic resistance gene). A vector containing a nucleic acid can encode a chimeric or fusion polypeptide (i.e., a polypeptide operatively linked to a heterologous polypeptide, which can be at either the N-terminus or C-terminus of the polypeptide). Representative heterologous polypeptides are those that can be used in purification of the encoded polypeptide (e.g., 6xHis tag, glutathione S-transferase (GST))

Expression elements include nucleic acid sequences that direct and regulate expression of nucleic acid coding sequences. One example of an expression element is a promoter sequence. Expression elements also can include introns, enhancer sequences, response elements, or inducible elements that modulate expression of a nucleic acid. Expression elements can be of bacterial, yeast, insect, mammalian, or viral origin, and vectors can contain a combination of elements from different origins. As used herein, operably linked means that a promoter or other expression element(s) are positioned in a vector relative to a nucleic acid in such a way as to direct or regulate expression of the nucleic acid (e.g., in-frame). Many methods for introducing nucleic acids into host cells, both in vivo and in vitro, are well known to those skilled in the art and include, without limitation, electroporation, calcium phosphate precipitation, polyethylene glycol (PEG) transformation, heat shock, lipofection, microinjection, and viral-mediated nucleic acid transfer.

Vectors as described herein can be introduced into a host cell. As used herein, “host cell” refers to the particular cell into which the nucleic acid is introduced and also includes the progeny of such a cell that carry the vector. A host cell can be any prokaryotic or eukaryotic cell. For example, nucleic acids can be expressed in bacterial cells such as E. coli, or in insect cells, yeast or mammalian cells (such as Chinese hamster ovary cells (CHO) or COS cells). Other suitable host cells are known to those skilled in the art.

Nucleic acids can be detected using any number of amplification techniques (see, e.g., PCR Primer: A Laboratory Manual, 1995, Dieffenbach & Dveksler, Eds., Cold Spring Harbor Laboratory Press, Cold Spring Harbor, NY; and U.S. Pat. Nos. 4,683,195; 4,683,202; 4,800,159; and 4,965,188) with an appropriate pair of oligonucleotides (e.g., primers). A number of modifications to the original PCR have been developed and can be used to detect a nucleic acid.

Nucleic acids also can be detected using hybridization. Hybridization between nucleic acids is discussed in detail in Sambrook et al. (1989, Molecular Cloning: A Laboratory Manual, 2nd Ed., Cold Spring Harbor Laboratory Press, Cold Spring Harbor, NY; Sections 7.37-7.57, 9.47-9.57, 11.7-11.8, and 11.45-11.57). Sambrook et al. discloses suitable Southern blot conditions for oligonucleotide probes less than about 100 nucleotides (Sections 11.45-11.46). The Tm between a sequence that is less than 100 nucleotides in length and a second sequence can be calculated using the formula provided in Section 11.46. Sambrook et al. additionally discloses Southern blot conditions for oligonucleotide probes greater than about 100 nucleotides (see Sections 9.47-9.54). The Tm between a sequence greater than 100 nucleotides in length and a second sequence can be calculated using the formula provided in Sections 9.50-9.51 of Sambrook et al.

The conditions under which membranes containing nucleic acids are prehybridized and hybridized, as well as the conditions under which membranes containing nucleic acids are washed to remove excess and non-specifically bound probe, can play a significant role in the stringency of the hybridization. Such hybridizations and washes can be performed, where appropriate, under moderate or high stringency conditions. For example, washing conditions can be made more stringent by decreasing the salt concentration in the wash solutions and/or by increasing the temperature at which the washes are performed. Simply by way of example, high stringency conditions typically include a wash of the membranes in 0.2×SSC at 65° C.

In addition, interpreting the amount of hybridization can be affected, for example, by the specific activity of the labeled oligonucleotide probe, by the number of probe-binding sites on the template nucleic acid to which the probe has hybridized, and by the amount of exposure of an autoradiograph or other detection medium. It will be readily appreciated by those of ordinary skill in the art that although any number of hybridization and washing conditions can be used to examine hybridization of a probe nucleic acid molecule to immobilized target nucleic acids, it is more important to examine hybridization of a probe to target nucleic acids under identical hybridization, washing, and exposure conditions. Preferably, the target nucleic acids are on the same membrane.

A nucleic acid molecule is deemed to hybridize to a nucleic acid but not to another nucleic acid if hybridization to a nucleic acid is at least 5-fold (e.g., at least 6-fold, 7-fold, 8-fold, 9-fold, 10-fold, 20-fold, 50-fold, or 100-fold) greater than hybridization to another nucleic acid. The amount of hybridization can be quantitated directly on a membrane or from an autoradiograph using, for example, a PhosphorImager or a Densitometer (Molecular Dynamics, Sunnyvale, CA).

Polypeptides can be detected using antibodies. Techniques for detecting polypeptides using antibodies include enzyme linked immunosorbent assays (ELISAs), Western blots, immunoprecipitations and immunofluorescence. An antibody can be polyclonal or monoclonal. An antibody having specific binding affinity for a polypeptide can be generated using methods well known in the art. The antibody can be attached to a solid support such as a microtiter plate using methods known in the art. In the presence of a polypeptide, an antibody-polypeptide complex is formed.

Detection (e.g., of an amplification product, a hybridization complex, or a polypeptide) is usually accomplished using detectable labels. The term “label” is intended to encompass the use of direct labels as well as indirect labels. Detectable labels include enzymes, prosthetic groups, fluorescent materials, luminescent materials, bioluminescent materials, and radioactive materials.

Certain nucleic acids described herein (e.g., SEQ ID NOs: 1, 3, 5, 7, or 82) are predicted to encode polypeptides (e.g., SEQ ID NOs: 2, 4, 6, 8, or 83) that belong to the nicotine uptake permease (Nup) family of sequences. Nup polypeptides are members of the larger family of purine permeases. See, for example, Hildreth et al., 2011, PNAS USA, 108:18279-84. In addition to the novel Nup nucleic acid and polypeptide sequences disclosed herein, representative Nup1 and Nup2 sequences from Nicotiana tabacum are shown in Accession Nos. GU174268.1 and GU174267.1.

Certain nucleic acids described herein (e.g., SEQ ID NOs: 9, 11, 13, 15, 70, 72, 74, 76, 78, or 80) are predicted to encode polypeptides (e.g., SEQ ID NOs: 10, 12, 14, 16, 71, 73, 75, 77, 79, or 81) that belong to the multiple drug resistance (MDR) family of sequences. Multidrug transporters form a large class of membrane proteins present in the cells of most organisms. These proteins bind to a variety of potentially cytotoxic compounds and remove them from the cell in an ATP- or proton-dependent process (Zhelenova et al., 2000, Trends Biochem. Sci., 25:39-43). Multidrug transporters previously have been divided into four superfamilies: the ATP binding cassette (ABC) superfamily, the major facilitator superfamily, the small multidrug resistance family, and the resistance-nodulation-cell division family (the MATE family discussed in more detail below was recently identified as a fifth superfamily of multidrug transporters). The efflux pump proteins which belong to the ATP-binding cassette superfamily and the major facilitator superfamily are the most prominent contributors to multidrug resistance (MDR).

Certain nucleic acids described herein (e.g., SEQ ID NOs: 17, 19, 21, 23, 25, 27, 29, 31, 86, 88, or 90) are predicted to encode polypeptides (e.g., SEQ ID NOs: 18, 20, 22, 24, 26, 28, 30, 87, 89, or 91) that belong to the multidrug and toxic compound extrusion-type (MATE) family of sequences. The MATE family of polypeptides is characterized by the presence of 12 putative transmembrane segments and by the absence of “signature sequences” specific to the other multidrug transporter superfamilies (Brown et al., 1999, Mol. Microbiol., 31:394-5). MATE proteins are believed to function as proton-dependent efflux transporters, and are abundant in bacteria and plants.

Certain nucleic acids described herein (e.g., SEQ ID NOs: 33 and 35) are predicted to encode polypeptides (e.g., SEQ ID NOs: 34 and 36) that belong to the pleiotropic drug resistance (PDR) family of sequences. See, for example, Moons, 2008, Planta, 229:53-71. The PDR family is only found in fungi and plants, and was first characterized in Saccharomyces cerevisiae by the increased expression of genes that encode for nonspecific drug-efflux transporter proteins. PDR polypeptides have been identified that confer resistance to a large set of functionally and structurally unrelated toxic compounds (e.g., antifungal and anticancer drugs) and that transport weak organic acids. In addition, a number of PDR genes have been identified in several plant species, including, for example, Arabidopsis and rice.

### Plants Having Reduced Amounts of Alkaloids in Leaf and Methods of Making

Tobacco hybrids, varieties, lines, or cultivars are provided that have a mutation in one or more endogenous nucleic acids described herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90). As described herein, leaf from plants having a mutation in one or more of the endogenous nucleic acids (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) can exhibit a reduced amount of at least one alkaloid (e.g., compared to leaf from a plant that lacks the mutation). In addition, leaf from plants having a mutation in one or more of the endogenous nucleic acids (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) can exhibit a reduced amount of at least one tobacco specific nitrosamine (TSNA) (e.g., compared to leaf from a plant lacking the mutation).

The alkaloids referred to herein are typically pyridine alkaloids, which includes nicotine, nornicotine, anabasine, myosmine, and anatabine. See, for example, Sheng et al., 2005, Chromatographia, 62:63-8. TSNAs are produced during curing. Nitrite may accumulate as a result of nitrate reduction by bacteria, and TSNAs are formed by chemical reactions between nitrite (source of nitrosating species) and alkaloids. Representative TSNAs include, for example, N′-nitrosonornicotine (NNN), 4-(methylnitrosoamino)-1-(3-pyridyl)-1-butanone (NNK), N′-nitrosoanatabine (NAT), N′-nitrosoanabasine (NAB), and 4-(methylnitrosoamino)-1-(3-pyridyl)-1-butanal (NNAL).

Methods of detecting alkaloids or TSNAs, and methods of determining the amount of one or more alkaloids or TSNAs are known in the art. For example, high performance liquid chromatography (HPLC)—mass spectroscopy (MS) (HPLC-MS) or high performance thin layer chromatography (HPTLC) can be used to detect the presence of one or more alkaloids and/or determine the amount of one or more alkaloids. In addition, any number of chromatography methods (e.g., gas chromatography/thermal energy analysis (GC/TEA), liquid chromatography/mass spectrometry (LC/MS), and ion chromatography (IC)) can be used to detect the presence of one or more TSNAs and/or determine the amount of one or more TSNAs.

Methods of making a tobacco plant having a mutation are known in the art. Mutations can be random mutations or targeted mutations. For random mutagenesis, cells (e.g., Nicotiana tabacum cells) can be mutagenized using, for example, a chemical mutagen, ionizing radiation, or fast neutron bombardment (see, e.g., Li et al., 2001, Plant J., 27:235-42). Representative chemical mutagens include, without limitation, nitrous acid, sodium azide, acridine orange, ethidium bromide, and ethyl methane sulfonate (EMS), while representative ionizing radiation includes, without limitation, x-rays, gamma rays, fast neutron irradiation, and UV irradiation. The dosage of the mutagenic chemical or radiation is determined experimentally for each type of plant tissue such that a mutation frequency is obtained that is below a threshold level characterized by lethality or reproductive sterility. The number of M₁ generation seed or the size of M₁ plant populations resulting from the mutagenic treatments are estimated based on the expected frequency of mutations. For targeted mutagenesis, representative technologies include TALEN (see, for example, Li et al., 2011, Nucleic Acids Res., 39(14):6315-25) or zinc-finger (see, for example, Wright et al., 2005, The Plant J., 44:693-705). Whether random or targeted, a mutation can be a point mutation, an insertion, a deletion, a substitution, or combinations thereof.

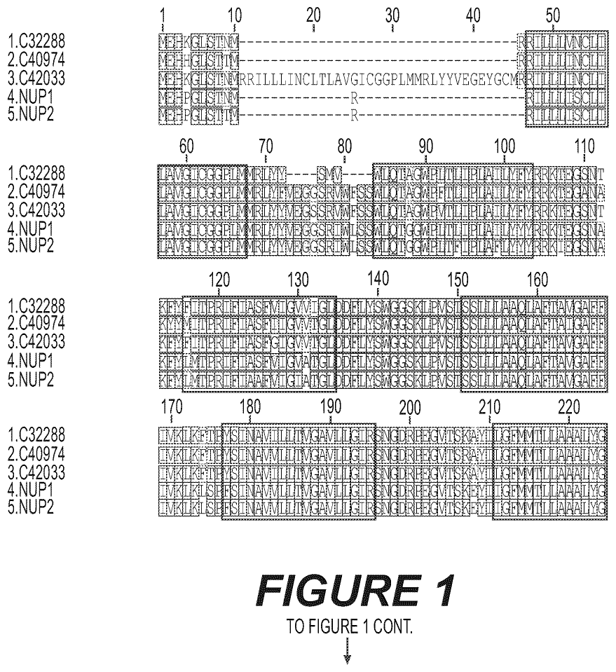

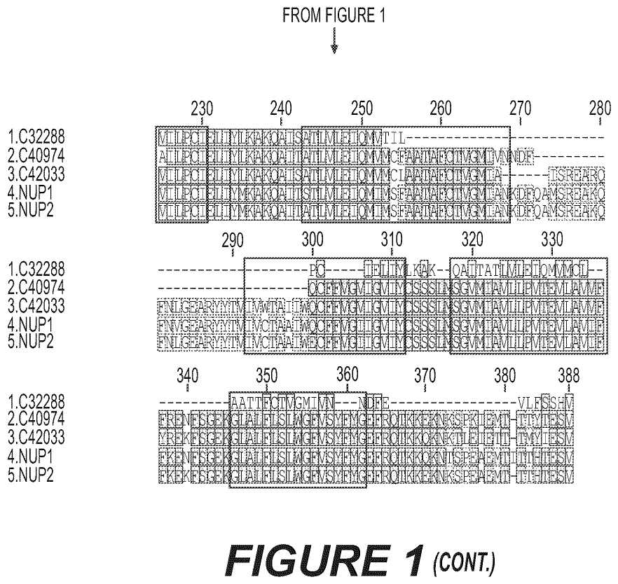

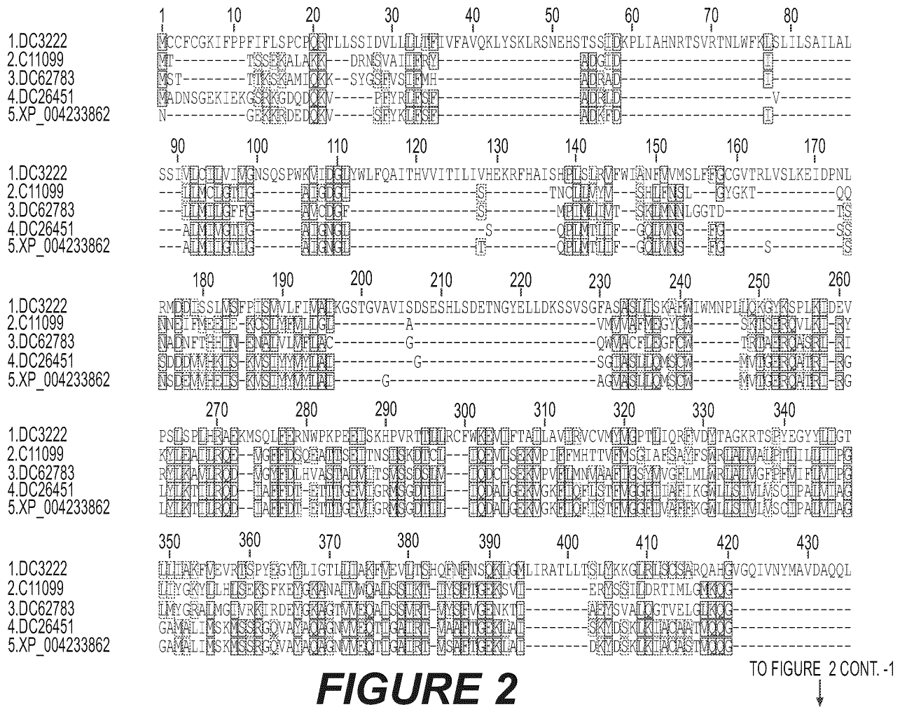

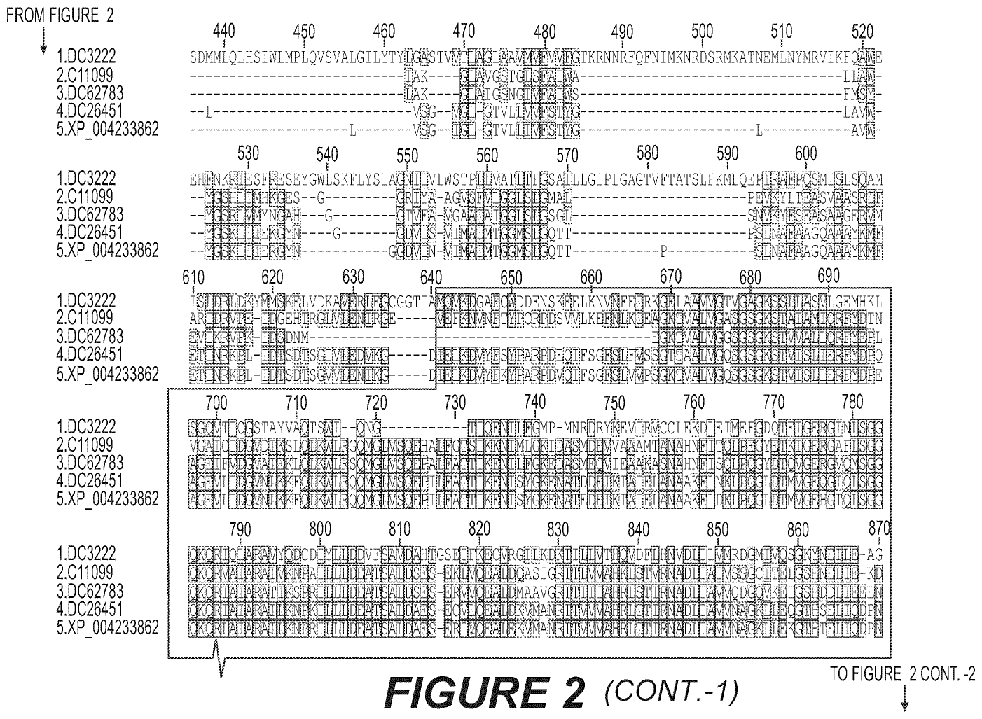

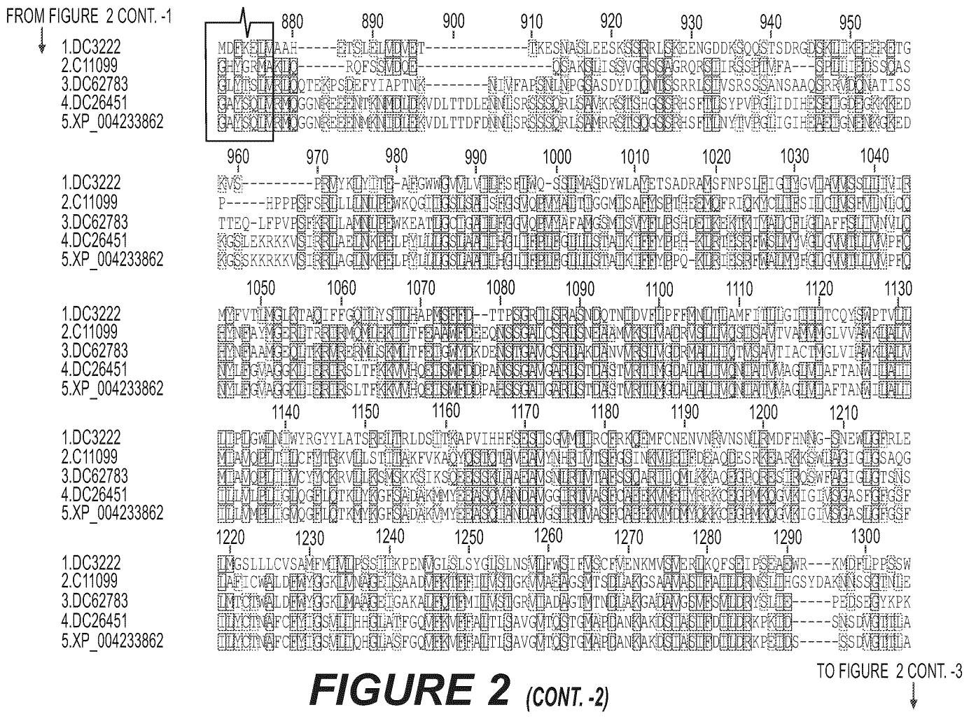

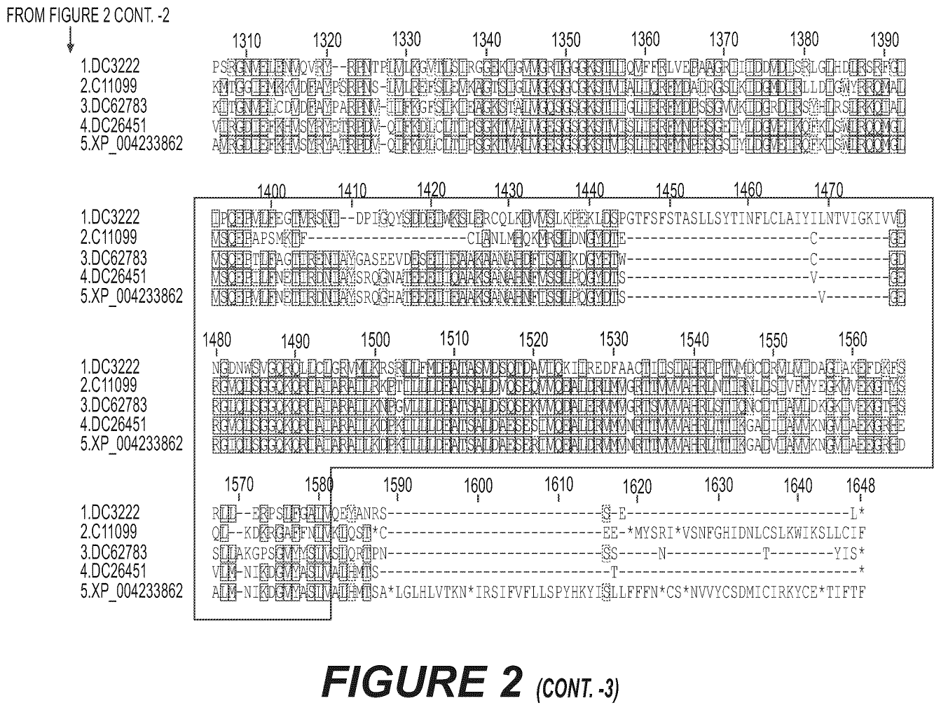

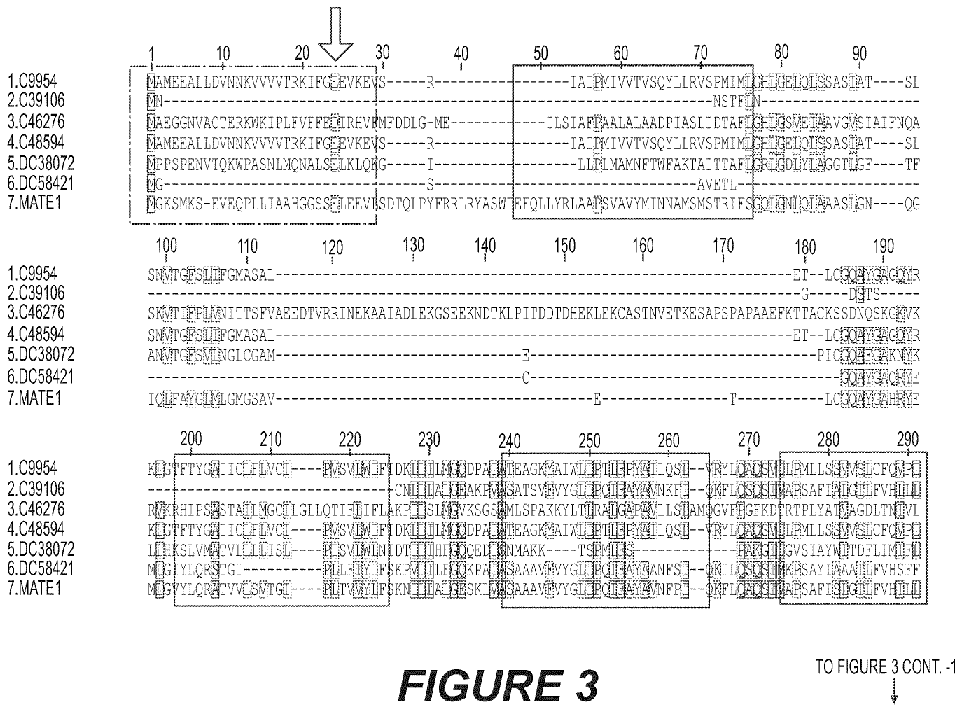

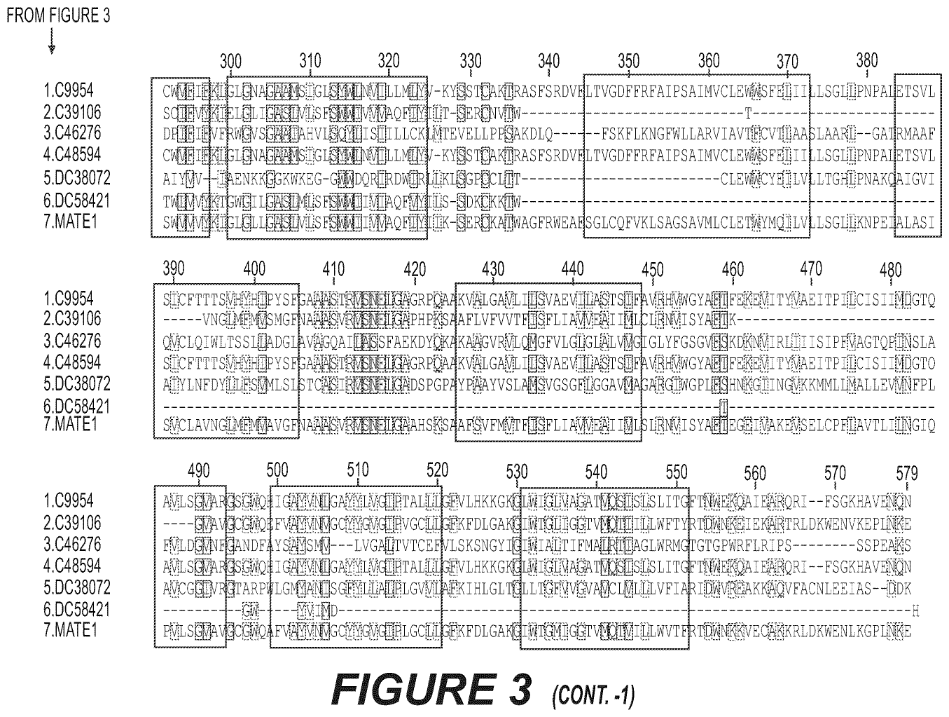

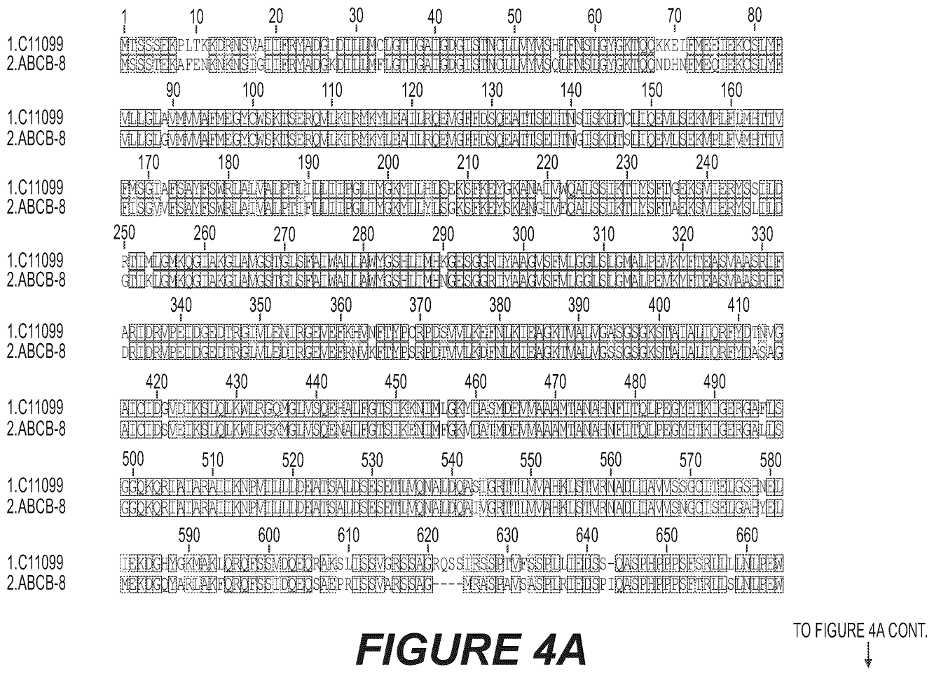

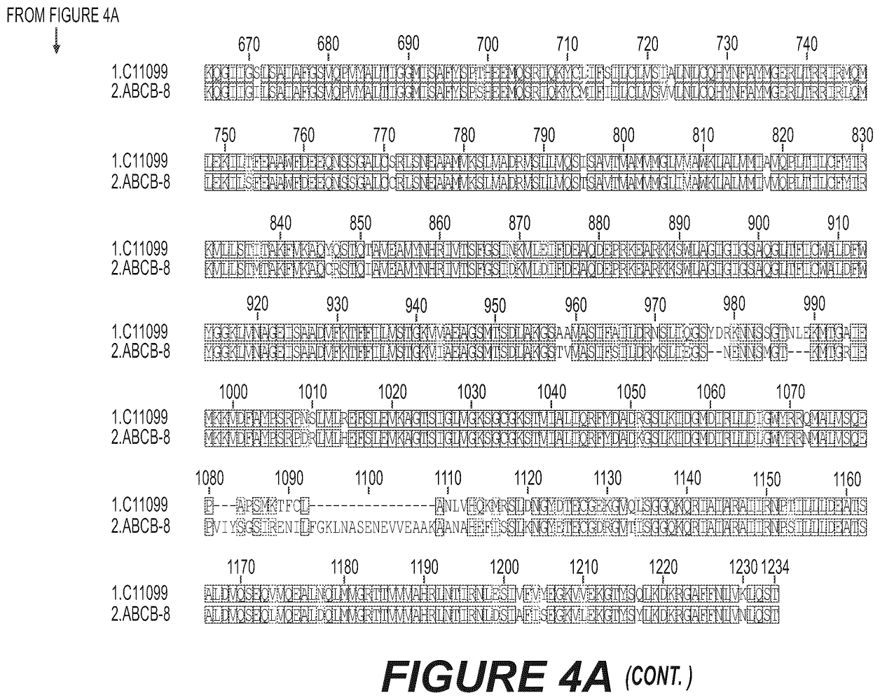

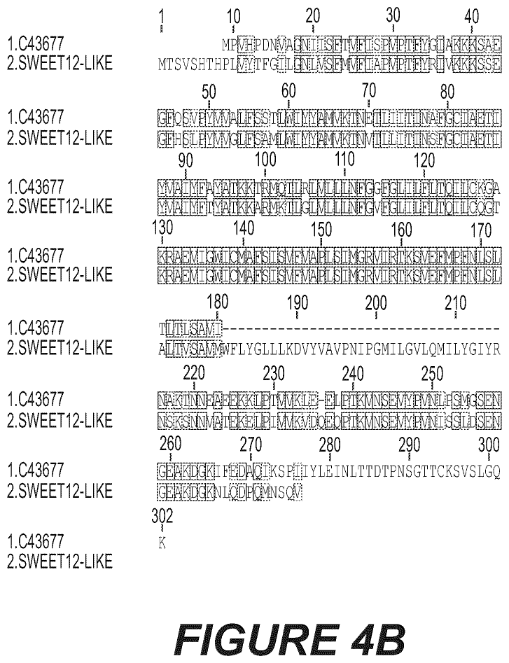

Conserved domains in polypeptides can be important for polypeptide function as well as cellular or subcellular location. FIG. 1 shows an alignment of Nup sequences, including the novel Nup sequences described herein, with the predicted transmembrane helices indicated by boxes. FIG. 2 shows an alignment of MDR sequences, including the novel MDR sequences described herein, with the conserved ATPase domains indicated by boxes. FIG. 3 shows an alignment of MATE sequences, including the novel MATE sequences described herein. The boxes in FIG. 3 indicate predicted transmembrane domains, with the predicted N-terminal signal peptide shown as shaded, and the predicted conserved cleavage site shown with an arrow. In addition, FIG. 4A shows an alignment between one of the novel MDR sequences, C11099, and a putative ABC transporter B family member 8-like sequence from Solanum lycopersicum. As indicated below in Table 5, these sequences have 84% sequence identity at the nucleotide level and 85% sequence identity at the amino acid level. Further, FIG. 4B shows an alignment between one of the novel sequence indicated as an “other” transporter sequence, C43677, and a bidirectional sugar transporter SWEET12-like sequence from Solanum lycopersicum. As indicated below in Table 8, these sequences have 71% sequence identity at the nucleotide level and 67% sequence identity at the amino acid level.

As discussed herein, one or more nucleotides can be mutated to alter the expression and/or function of the encoded polypeptide, relative to the expression and/or function of the corresponding wild type polypeptide. It will be appreciated, for example, that a mutation in one or more of the highly conserved regions (see, for example, the alignments shown in FIGS. 1, 2, 3, and 4) would likely alter polypeptide function, while a mutation outside of those conserved regions would likely have little to no effect on polypeptide function. In addition, a mutation in a single nucleotide can create a stop codon, which would result in a truncated polypeptide and, depending on the extent of truncation, loss-of-function.

Preferably, a mutation in one of the novel nucleic acids disclosed herein results in reduced or even complete elimination of transporter activity in a tobacco plant comprising the mutation. Suitable types of mutations in a transporter coding sequence include, without limitation, insertions of nucleotides, deletions of nucleotides, or transitions or transversions in the wild-type transporter coding sequence. Mutations in the coding sequence can result in insertions of one or more amino acids, deletions of one or more amino acids, and/or non-conservative amino acid substitutions in the encoded polypeptide. In some cases, the coding sequence of a transporter comprises more than one mutation or more than one type of mutation.

Insertion or deletion of amino acids in a coding sequence, for example, can disrupt the conformation of the encoded polypeptide. Amino acid insertions or deletions also can disrupt sites important for recognition of the binding ligand (i.e., the molecule(s) that are transported) or for activity of the polypeptide (i.e., transporter activity). It is known in the art that the insertion or deletion of a larger number of contiguous amino acids is more likely to render the gene product non-functional, compared to a smaller number of inserted or deleted amino acids. In addition, one or more mutations (e.g., a point mutation) can change the localization of the transporter polypeptide, introduce a stop codon to produce a truncated polypeptide, or disrupt an active site or domain (e.g., a catalytic site or domain, a binding site or domain) within the polypeptide.

Simply by way of example, a MATE transporter sequence (e.g., FIG. 3; e.g., C9954 (SEQ ID NO:26), C46276 (SEQ ID NO:22), C48594 (SEQ ID NO:24), and DC38072 (SEQ ID NO:20)) can be mutated to change the charged amino acid at residue 24 to an uncharged amino acid. Such a mutation can disrupt the usual targeting of the transport polypeptide to the cell surface, which can alter the transport of one or more alkaloids within the plant (e.g., into the xylem and/or from the root to the leaf). In addition, a MDR transporter (e.g., C11099 (SEQ ID NO:16)) can be mutated to change the T at nucleotide 124 to an A, which would result in a stop codon after the eighth amino acid residue. Such a mutation would significantly reduce or essentially eliminate the transporter polypeptide in the plant; a mutation to introduce a stop codon can be similarly applied to any of the transporter sequences disclosed herein. Further, the MDR (e.g., FIGS. 2 and 4A) and PDR (e.g., SEQ ID NOs: 33-36) family of polypeptides require hydrolysis of ATP for transport; ATPase domains are highly conserved and the amino acid residues required for hydrolysis are known (e.g., the Walker A amino acid motif (GXXGXGK) at residues 676-682 of FIG. 2). Thus, a MDR polypeptide (e.g., DC3222 (SEQ ID NO:10), C11099 (SEQ ID NO:16), DC62783 (SEQ ID NO:12), and DC26451 (SEQ ID NO:14)) or a PDR polypeptide (e.g., C53160 (SEQ ID NO:34), C22474 (SEQ ID NO:36)) can be mutated within the Walker A motif (e.g., at the conserved lysine (K) amino acid), which would result in a polypeptide that is unable to hydrolyze ATP and unable to perform, or at least deficient in, its ability to transport.

Non-conservative amino acid substitutions can replace an amino acid of one class with an amino acid of a different class. Non-conservative substitutions can make a substantial change in the charge or hydrophobicity of the gene product. Non-conservative amino acid substitutions can also make a substantial change in the bulk of the residue side chain, e.g., substituting an alanine residue for an isoleucine residue. Examples of non-conservative substitutions include a basic amino acid for a non-polar amino acid, or a polar amino acid for an acidic amino acid.

Transmembrane polypeptides such as transporter polypeptides contain particular sequences that determine where the polypeptide is localized within the cell. For example, while the previously described MATE1 protein contains sequences that target it to the vacuole, the novel MATE sequences described here have different N terminal domains (see the alignment in FIG. 3). The target peptide sequences often are cleaved (e.g., by specific proteases that recognize a specific nucleotide motif) after the polypeptide is inserted into the membrane. By mutating the target sequence or a cleavage motif, the targeting of the polypeptide can be altered.

Following mutagenesis, M₀ plants are regenerated from the mutagenized cells and those plants, or a subsequent generation of that population (e.g., M₁, M₂, M₃, etc.), can be screened for a mutation in a sequence of interest (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90). Screening for plants carrying a mutation in a sequence of interest can be performed using methods routine in the art (e.g., hybridization, amplification, combinations thereof) or by evaluating the phenotype (e.g., detecting and/or determining the amount of one or more alkaloids and/or one or more TSNAs in the roots and/or the leaf). Generally, the presence of a mutation in one or more of the nucleic acid sequences disclosed herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) results in a reduction of one or more alkaloids in the leaf of the mutant plants and/or one or more TSNAs in the cured leaf of the mutant plants compared to a corresponding plant (e.g., having the same varietal background) lacking the mutation.

As used herein, “reduced” or “reduction” refers to a decrease (e.g., a statistically significant decrease) in the amount of one or more alkaloids in tobacco leaf, either green or cured, and/or one or more TSNAs in green or cured leaf by at least about 5% up to about 95% (e.g., about 5% to about 10%, about 5% to about 20%, about 5% to about 50%, about 5% to about 75%, about 10% to about 25%, about 10% to about 50%, about 10% to about 90%, about 20% to about 40%, about 20% to about 60%, about 20% to about 80%, about 25% to about 75%, about 50% to about 75%, about 50% to about 85%, about 50% to about 95%, and about 75% to about 95%) relative to similarly-treated leaf (e.g., green or cured) from a tobacco plant lacking the mutation. As used herein, statistical significance refers to a p-value of less than 0.05, e.g., a p-value of less than 0.025 or a p-value of less than 0.01, using an appropriate measure of statistical significance, e.g., a one-tailed two sample t-test.

An M₁ tobacco plant may be heterozygous for a mutant allele and exhibit a wild type phenotype. In such cases, at least a portion of the first generation of self-pollinated progeny of such a plant exhibits a wild type phenotype. Alternatively, an M₁ tobacco plant may have a mutant allele and exhibit a mutant phenotype. Such plants may be heterozygous and exhibit a mutant phenotype due to a phenomenon such as dominant negative suppression, despite the presence of the wild type allele, or such plants may be homozygous due to independently induced mutations in both alleles.

A tobacco plant carrying a mutant allele can be used in a plant breeding program to create novel and useful cultivars, lines, varieties and hybrids. Thus, in some embodiments, an M₁, M₂, M₃ or later generation tobacco plant containing at least one mutation is crossed with a second Nicotiana tabacum plant, and progeny of the cross are identified in which the mutation(s) is present. It will be appreciated that the second Nicotiana tabacum plant can be one of the species and varieties described herein. It will also be appreciated that the second Nicotiana tabacum plant can contain the same mutation as the plant to which it is crossed, a different mutation, or be wild type at the locus. Additionally or alternatively, a second tobacco line can exhibit a phenotypic trait such as, for example, disease resistance, high yield, high grade index, curability, curing quality, mechanical harvesting, holding ability, leaf quality, height, plant maturation (e.g., early maturing, early to medium maturing, medium maturing, medium to late maturing, or late maturing), stalk size (e.g., small, medium, or large), and/or leaf number per plant (e.g., a small (e.g., 5-10 leaves), medium (e.g., 11-15 leaves), or large (e.g., 16-21) number of leaves).

Breeding is carried out using known procedures. DNA fingerprinting, SNP or similar technologies may be used in a marker-assisted selection (MAS) breeding program to transfer or breed mutant alleles into other tobaccos, as described herein. Progeny of the cross can be screened for a mutation using methods described herein, and plants having a mutation in a nucleic acid sequence disclosed herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) can be selected. For example, plants in the F₂ or backcross generations can be screened using a marker developed from a sequence described herein or a fragment thereof, using one of the techniques listed herein. Leaf (green or cured, as appropriate) from progeny plants also can be screened for the amount of one or more alkaloids and/or one or more TSNAs, and those plants having reduced amounts, compared to a corresponding plant that lacks the mutation, can be selected. Plants identified as possessing the mutant allele and/or the mutant phenotype can be backcrossed or self-pollinated to create a second population to be screened. Backcrossing or other breeding procedures can be repeated until the desired phenotype of the recurrent parent is recovered.

Successful crosses yield F₁ plants that are fertile and that can be backcrossed with one of the parents if desired. In some embodiments, a plant population in the F₂ generation is screened for the mutation or variant gene expression using standard methods (e.g., PCR with primers based upon the nucleic acid sequences disclosed herein). Selected plants are then crossed with one of the parents and the first backcross (BC₁) generation plants are self-pollinated to produce a BC₁F₂ population that is again screened for variant gene expression. The process of backcrossing, self-pollination, and screening is repeated, for example, at least four times until the final screening produces a plant that is fertile and reasonably similar to the recurrent parent. This plant, if desired, is self-pollinated and the progeny are subsequently screened again to confirm that the plant contains the mutation and exhibits variant gene expression. Breeder's seed of the selected plant can be produced using standard methods including, for example, field testing, confirmation of the null condition, and/or chemical analyses of leaf (e.g., cured leaf) to determine the level of alkaloids.

The result of a plant breeding program using the mutant tobacco plants described herein are novel and useful cultivars, varieties, lines, and hybrids. As used herein, the term “variety” refers to a population of plants that share constant characteristics which separate them from other plants of the same species. A variety is often, although not always, sold commercially. While possessing one or more distinctive traits, a variety is further characterized by a very small overall variation between individual with that variety. A “pure line” variety may be created by several generations of self-pollination and selection, or vegetative propagation from a single parent using tissue or cell culture techniques. A “line,” as distinguished from a variety, most often denotes a group of plants used non-commercially, for example, in plant research. A line typically displays little overall variation between individuals for one or more traits of interest, although there may be some variation between individuals for other traits.

A variety can be essentially derived from another line or variety. As defined by the International Convention for the Protection of New Varieties of Plants (Dec. 2, 1961, as revised at Geneva on Nov. 10, 1972, On Oct. 23, 1978, and on Mar. 19, 1991), a variety is “essentially derived” from an initial variety if: a) it is predominantly derived from the initial variety, or from a variety that is predominantly derived from the initial variety, while retaining the expression of the essential characteristics that result from the genotype or combination of genotypes of the initial variety; b) it is clearly distinguishable from the initial variety; and c) except for the differences which result from the act of derivation, it confirms to the initial variety in the expression of the essential characteristics that result from the genotype or combination of genotypes of the initial variety. Essentially derived varieties can be obtained, for example, by the selection of a natural or induced mutant, a somaclonal variant, a variant individual plant from the initial variety, backcrossing, or transformation.

Tobacco hybrids can be produced by preventing self-pollination of female parent plants (i.e., seed parents) of a first variety, permitting pollen from male parent plants of a second variety to fertilize the female parent plants, and allowing F₁ hybrid seeds to form on the female plants. Self-pollination of female plants can be prevented by emasculating the flowers at an early stage of flower development. Alternatively, pollen formation can be prevented on the female parent plants using a form of male sterility. For example, male sterility can be produced by cytoplasmic male sterility (CMS), nuclear male sterility, genetic male sterility, molecular male sterility wherein a transgene inhibits microsporogenesis and/or pollen formation, or self-incompatibility. Female parent plants containing CMS are particularly useful. In embodiments in which the female parent plants are CMS, the male parent plants typically contain a fertility restorer gene to ensure that the F₁ hybrids are fertile. In other embodiments in which the female parents are CMS, male parents can be used that do not contain a fertility restorer. F₁ hybrids produced from such parents are male sterile. Male sterile hybrid seed can be interplanted with male fertile seed to provide pollen for seed-set on the resulting male sterile plants.

Varieties, lines and cultivars described herein can be used to form single-cross tobacco F₁ hybrids. In such embodiments, the plants of the parent varieties can be grown as substantially homogeneous adjoining populations to facilitate natural cross-pollination from the male parent plants to the female parent plants. The F₂ seed formed on the female parent plants is selectively harvested by conventional means. One also can grow the two parent plant varieties in bulk and harvest a blend of F₁ hybrid seed formed on the female parent and seed formed upon the male parent as the result of self-pollination. Alternatively, three-way crosses can be carried out wherein a single-cross F₁ hybrid is used as a female parent and is crossed with a different male parent. As another alternative, double-cross hybrids can be created wherein the F₁ progeny of two different single-crosses are themselves crossed. Self-incompatibility can be used to particular advantage to prevent self-pollination of female parents when forming a double-cross hybrid.

The tobacco plants used in the methods described herein can be a Burley type, a dark type, a flue-cured type, a Maryland type, or an Oriental type. The tobacco plants used in the methods described herein typically are from N. tabacum, and can be from any number of N. tabacum varieties. A variety can be BU 64, CC 101, CC 200, CC 13, CC 27, CC 33, CC 35, CC 37, CC 65, CC 67, CC 301, CC 400, CC 500, CC 600, CC 700, CC 800, CC 900, CC 1063, Coker 176, Coker 319, Coker 371 Gold, Coker 48, CU 263, DF911, Galpao tobacco, GL 26H, GL 338, GL 350, GL 395, GL 600, GL 737, GL 939, GL 973, GF 157, GF 318, RJR 901, HB 04P, K 149, K 326, K 346, K 358, K394, K 399, K 730, NC 196, NC 37NF, NC 471, NC 55, NC 92, NC2326, NC 95, NC 925, PVH 1118, PVH 1452, PVH 2110, PVH 2254, PVH 2275, VA 116, VA 119, KDH 959, KT 200, KT204LC, KY 10, KY 14, KY 160, KY 17, KY 171, KY 907, KY907LC, KTY14×L8 LC, Little Crittenden, McNair 373, McNair 944, msKY 14×L8, Narrow Leaf Madole, NC 100, NC 102, NC 2000, NC 291, NC 297, NC 299, NC 3, NC 4, NC 5, NC 6, NC7, NC 606, NC 71, NC 72, NC 810, NC BH 129, NC 2002, Neal Smith Madole, OXFORD 207, ‘Perique’ tobacco, PVH03, PVH09, PVH19, PVH50, PVH51, R 610, R 630, R 7-11, R 7-12, RG 17, RG 81, RG H51, RGH 4, RGH 51, RS 1410, Speight 168, Speight 172, Speight 179, Speight 210, Speight 220, Speight 225, Speight 227, Speight 234, Speight G-28, Speight G-70, Speight H-6, Speight H20, Speight NF3, TI 1406, TI 1269, TN 86, TN86LC, TN 90, TN90LC, TN 97, TN97LC, TN D94, TN D950, TR (Tom Rosson) Madole, VA 309, or VA359.

In addition to mutation, another way in which the amount of alkaloids in tobacco leaf can be reduced is to use inhibitory RNAs (e.g., RNAi). Therefore, transgenic tobacco plants are provided that contain a transgene encoding at least one RNAi molecule, which, when expressed, silences at least one of the endogenous nucleic acids described herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90). As described herein, leaf from such transgenic plants exhibit a reduced amount of at least one alkaloid (e.g., compared to leaf from a plant lacking or not expressing the RNAi). In addition, leaf from such transgenic plants exhibit a reduced amount of at least one tobacco specific nitrosamine (TSNA) (e.g., compared to leaf from a plant lacking or not expressing the RNAi).

RNAi technology is known in the art and is a very effective form of post-transcriptional gene silencing. RNAi molecules typically contain a nucleotide sequence (e.g., from about 18 nucleotides in length (e.g., about 19 or 20 nucleotides in length) up to about 700 nucleotides in length) that is complementary to the target gene in both the sense and antisense orientations. The sense and antisense strands can be connected by a short “loop” sequence (e.g., about 5 nucleotides in length up to about 800 nucleotides in length) and expressed in a single transcript, or the sense and antisense strands can be delivered to and expressed in the target cells on separate vectors or constructs. A number of companies offer RNAi design and synthesis services (e.g., Life Technologies, Applied Biosystems), and representative RNAi molecules to a number of the novel sequences described herein are provided in SEQ ID NOs: 51-56.

The RNAi molecule can be expressed using a plant expression vector. The RNAi molecule typically is at least 25 nucleotides in length and has at least 91% sequence identity (e.g., at least 95%, 96%, 97%, 98% or 99% sequence identity) to one of the nucleic acid sequences disclosed herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) or hybridizes under stringent conditions to one of the nucleic acid sequences disclosed herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90). Hybridization under stringent conditions is described above.

Methods of introducing a nucleic acid (e.g., a heterologous nucleic acid) into plant cells are known in the art and include, for example, particle bombardment, Agrobacterium-mediated transformation, microinjection, polyethylene glycol-mediated transformation (e.g., of protoplasts, see, for example, Yoo et al. (2007, Nature Protocols, 2(7):1565-72)), liposome-mediated DNA uptake, or electroporation. Following transformation, the transgenic plant cells can be regenerated into transgenic tobacco plants. As described herein, expression of the transgene results in leaf that exhibits a reduced amount of at least one alkaloid and/or at least one TSNA in the resulting cured leaf relative to leaf from a plant not expressing the transgene. The leaves of the regenerated transgenic plants can be screened for the amount of one or more alkaloids and/or one or more TSNAs in the resulting cured leaf, and plants having reduced amounts of at least one alkaloid and/or at least one TSNA in the resulting cured leaf, compared to the amount in a corresponding non-transgenic plant, can be selected for use in, for example, a breeding program as discussed herein.

Nucleic acids that confer traits such as herbicide resistance (sometimes referred to as herbicide tolerance), insect resistance, or stress tolerance, can also be present in the novel tobacco plants described herein. Genes conferring resistance to a herbicide that inhibits the growing point or meristem, such as an imidazolinone or a sulfonylurea, can be suitable. Exemplary genes in this category encode mutant ALS and AHAS enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS), which is resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides.

Genes for resistance to glyphosate also are suitable. See, for example, U.S. Pat. Nos. 4,940,835 and 4,769,061. Such genes can confer resistance to glyphosate herbicidal compositions, including, without limitation, glyphosate salts such as the trimethylsulphonium salt, the isopropylamine salt, the sodium salt, the potassium salt and the ammonium salt. See, e.g., U.S. Pat. Nos. 6,451,735 and 6,451,732. Genes for resistance to phosphono compounds such as glufosinate ammonium or phosphinothricin, and pyridinoxy or phenoxy propionic acids and cyclohexones also are suitable. See, e.g., U.S. Pat. Nos. 5,879,903; 5,276,268; and 5,561,236; and European Application No. 0 242 246.

Other suitable herbicides include those that inhibit photosynthesis, such as a triazine and a benzonitrile (nitrilase). See U.S. Pat. No. 4,810,648. Other suitable herbicides include 2,2-dichloropropionic acid, sethoxydim, haloxyfop, imidazolinone herbicides, sulfonylurea herbicides, triazolopyrimidine herbicides, s-triazine herbicides and bromoxynil. Also suitable are herbicides that confer resistance to a protox enzyme. See, e.g., U.S. Pat. No. 6,084,155 and US 20010016956.

A number of genes are available that confer resistance to insects, for example, insects in the order Lepidoptera. Exemplary genes include those that encode truncated Cry1A(b) and Cry1A(c) toxins. See, e.g., genes described in U.S. Pat. Nos. 5,545,565; 6,166,302; and 5,164,180. See also, Vaeck et al., 1997, Nature, 328:33-37 and Fischhoff et al., 1987, Nature Biotechnology, 5:807-813. Particularly useful are genes encoding toxins that exhibit insecticidal activity against Manduca sexta (tobacco hornworm); Heliothis virescens Fabricius (tobacco budworm) and/or S. litura Fabricius (tobacco cutworm).

### Plants Having Increased Amounts of Alkaloids in Leaf and Methods of Making

The sequences described herein can be overexpressed in plants in order to increase the amount of one or more alkaloids (and/or one or more TSNAs) in the leaf. Therefore, transgenic tobacco plants, or leaf from such plants, are provided that are transformed with a nucleic acid molecule described herein (e.g., SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) or a functional fragment thereof under control of a promoter that is able to drive expression in plants (e.g., a plant promoter). As discussed herein, a nucleic acid molecule used in a plant expression vector can have a different sequence than a sequence described herein, which can be expressed as a percent sequence identity (e.g., relative to SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90) or based on the conditions under which the sequence hybridizes to SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, or 90.

As an alternative to using a full-length sequence, a portion of the sequence can be used that encodes a polypeptide fragment having the desired functionality (referred to herein as a “functional fragment”). When used with respect to nucleic acids, it would be appreciated that it is not the nucleic acid fragment that possesses functionality but the encoded polypeptide fragment. Based on the disclosure herein and the alignments shown in FIGS. 1, 2, 3 and 4, one of skill in the art can predict the portion(s) of a polypeptide (e.g., one or more domains) that may impart the desired functionality.

Following transformation, the transgenic tobacco cells can be regenerated into transgenic tobacco plants. The leaves of the regenerated tobacco plants can be screened for the amount of one or more alkaloids, and plants having increased amounts of at least one alkaloid, compared to the amount in a corresponding non-transgenic plant, can be selected and used, for example, in a breeding program as discussed herein. Expression of the nucleic acid molecule or a functional fragment thereof may result in leaf that exhibits an increased amount of at least one alkaloid compared to leaf from a tobacco plant that does not express the nucleic acid molecule or functional fragment thereof. Nucleic acids conferring herbicide resistance, insect resistance, or stress tolerance, can also be introduced into such tobacco plants.

### Tobacco Products and Methods of Making

The methods described herein allow for leaf constituents in a tobacco plant to be altered. As described herein, altering leaf constituents refers to reducing or increasing the amount of at least one alkaloid in the leaf. As described herein, such methods can include mutagenesis (e.g., random or targeted) or the production of transgenic plants (using, e.g., RNAi or overexpression).

Leaf from such tobacco (e.g., having reduced or increased amounts of one or more alkaloids) can be cured, aged, conditioned, and/or fermented. Methods of curing tobacco are well known and include, for example, air curing, fire curing, flue curing and sun curing. Aging also is known and typically is carried out in a wooden drum (e.g., a hogshead) or cardboard cartons in compressed conditions for several years (e.g., 2 to 5 years), at a moisture content of from about 10% to about 25% (see, for example, U.S. Pat. No. 4,516,590 and 5,372,149). Conditioning includes, for example, a heating, sweating or pasteurization step as described in US 2004/0118422 or US 2005/0178398, while fermenting typically is characterized by high initial moisture content, heat generation, and a 10 to 20% loss of dry weight. See, e.g., U.S. Pat. Nos. 4,528,993, 4,660,577, 4,848,373 and 5,372,149. The tobacco also can be further processed (e.g., cut, expanded, blended, milled or comminuted), if desired, and used in a tobacco product.

Tobacco products are known in the art and include any product made or derived from tobacco that is intended for human consumption, including any component, part, or accessory of a tobacco product. Representative tobacco products include, without limitation, smokeless tobacco products, tobacco-derived nicotine products, cigarillos, non-ventilated recess filter cigarettes, vented recess filter cigarettes, cigars, snuff, pipe tobacco, cigar tobacco, cigarette tobacco, chewing tobacco, leaf tobacco, shredded tobacco, and cut tobacco. Representative smokeless tobacco products include, for example, chewing tobacco, snus, pouches, films, tablets, coated dowels, rods, and the like. Representative cigarettes and other smoking articles include, for example, smoking articles that include filter elements or rod elements, where the rod element of a smokeable material can include cured tobacco within a tobacco blend. In addition to the reduced-alkaloid tobacco described herein, tobacco products also can include other ingredients such as, without limitation, binders, plasticizers, stabilizers, and/or flavorings. See, for example, US 2005/0244521, US 2006/0191548, US 2012/0024301, US 2012/0031414, and US 2012/0031416 for examples of tobacco products.

The invention will be further described in the following examples, which do not limit the scope of the methods and compositions of matter described in the claims.

### EXAMPLES

### Example 1—Transport of Alkaloids from Root to Leaf

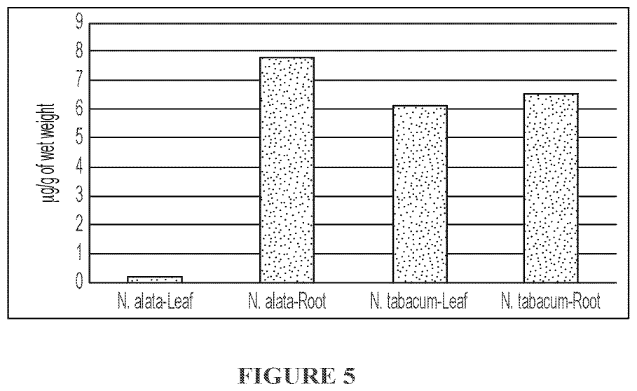

Previous studies have shown that Nicotiana alata, a relative of Nicotiana tabacum, does not transport alkaloids to the leaves (Pakdeechanuan et al., 2012, Plant Cell Physiol., 53(7):1247-54). This phenotype was confirmed as shown in FIG. 5. The possibility that N. alata is able to transport alkaloids after topping was also tested. See Table 1. Under no condition tested were alkaloids found to be transported to the leaf.

TABLE 1
Alkaloid content of N. alata leaves and roots with and without topping

| | Nicotine | Nornicotine | Anabasine | Myosmine | Anatabine |
|---|---|---|---|---|---|
| Code | (μg/ml) | (μg/ml) | (μg/ml) | (μg/ml) | (μg/ml) |
| Leaf from | <LOQ | ND | ND | ND | ND |
| topped plants | | | | | |
| Root from | 3.85 | 0.270 | <LOQ | 0.108 | 0.277 |
| topped plants | | | | | |
| Leaf from | <LOQ | ND | ND | ND | ND |
| untopped plants | | | | | |
| Root from | 2.52 | 0.186 | ND | 0.0858 | <LOQ |
| untopped plants | | | | | |
| Approx. limit of | 0.973 | 0.040 | 0.012 | 0.00823 | 0.128 |
| quantitation | | | | | |

LOQ, level of quantification;
ND, not detected

### Example 2—RNA Preparation and Sequencing

RNA from root tissue of N. alata plants before and after topping was collected and RNA-sequencing libraries were created using the True-Seq Library Construction Kit from Illumina. Sequencing of N. alata RNA was accomplished using the Illumina MiSeq platform. Sequencing runs were conducted with 150 cycle paired end read parameters, and library quality and average size were determined using an E-Gene capillary electrophoresis instrument. Root-specific gene expression in TN90 tobacco was determined by RNA deep sequencing performed by ArrayXpress (Raleigh, NC).

After sequencing, the resultant sequences were trimmed and reads with quality scores above 30 were assembled into contigs. Individual sequence reads were then mapped onto these contigs. Sequencing Run 1 generated 4.6 million mapped reads for a total of 69 Mb of sequence data, and Sequencing Run 2 generated 2.5 million mapped reads for a total of 37.5 Mb of sequence data. This resulted in 106.5 Mb of sequence reads used in the analysis of N. alata gene expression.

Full length coding sequences were determined by comparison to a N. benthamiana reference genome with a cutoff of 95% sequence identity. The full length coding sequences and the predicted polypeptides encoded thereby are shown in SEQ ID NOs:1-50 and 70-91.

### Example 3—Analysis of Expression Levels

The TN90 and N. alata gene expression data were analyzed to identify transport related genes that are differentially expressed or undetected in N. alata compared to N. tabacum. The TN90 expression data was filtered for root specific expression based on differences in expression from other tissues (>9 fold higher gene expression, p-value<0.000001). Results were then filtered for high root expression (>100 reads after topping). Genes were then filtered for gene ontology (GO) terms that denote transporters of secondary metabolites (e.g., the term “transport” coupled with “drug” or “purine” were used to filter the results). Genes that were not detected in N. alata in the two independent sequencing runs were filtered using the same GO criteria. These datasets then were compared to determine genes that fall into all three categories (i.e., (i) high root specific expression, (ii) GO criteria, and (iii) not expressed in N. alata).

These genes are listed, along with their expression level in TN90 tobacco, in Table 2.

TABLE 2
Gene expression in TN90

| | | | | | | | | | SEQ ID |
|---|---|---|---|---|---|---|---|---|---|
| | Gene | Root | Root | Root | | | | N. | NO |
| | Designation | (0 hr*) | (24 hr*) | (72 hr*) | Bud | Leaf | SEM | alata | (nt/prot) |
| NUP | C32288 | 495 | 229 | 359 | 13 | 25 | 23 | ND | 1/2 |
| | C40974 | 820 | 129 | 184 | 3 | 6 | 4 | ND | 3/4 |
| | C42033 | 382 | 91 | 147 | 16 | 12 | 15 | ND | 5/6 |
| | C29462 | 769 | 459 | 414 | 42 | 119 | 61 | ND | 7/8 |
| MDR | DC3222 | 224 | 296 | 180 | 759 | 2332 | 1058 | ND | 9/10 |
| | DC62783 | 255 | 225 | 176 | 7 | 4 | 2 | ND | 11/12 |
| | DC26451 | 192 | 205 | 174 | 14 | 67 | 11 | ND | 13/14 |
| | C11099 | 34 | 198 | 158 | 1 | 0 | 1 | ND | 15/16 |
| MATE | DC58421 | 149 | 320 | 221 | 15 | 16 | 5 | ND | 17/18 |
| | DC38072 | 153 | 134 | 147 | 11 | 39 | 12 | ND | 19/20 |
| | C46276 | 70 | 658 | 422 | 5 | 4 | 4 | ND | 21/22 |
| | C48594 | 188 | 307 | 321 | 9 | 3 | 9 | ND | 23/24 |
| | C9954 | 91 | 154 | 181 | 4 | 3 | 3 | ND | 25/26 |
| | C39106 | 308 | 502 | 527 | 12 | 9 | 10 | ND | 27/28 |
| | C3055 | 416 | 681 | 512 | 11 | 122 | 6 | ND | 29/30 |
| | DC14012 | 209 | 178 | 208 | 525 | 447 | 459 | ND | 31/32 |
| PDR | C53160 | 984 | 947 | 772 | 149 | 175 | 127 | ND | 33/34 |
| | C22474 | 916 | 906 | 599 | 84 | 76 | 64 | ND | 35/36 |
| Other | DC69629 | 267 | 396 | 480 | 17 | 5 | 10 | ND | 37/38 |
| | C43677 | 17 | 17 | 116 | 243 | 9 | 127 | ND | 39/40 |
| | C19125 | 138 | 252 | 177 | 8 | 32 | 17 | ND | 41/42 |

*hrs after topping;
ND = not detected

The expression level of N. alata genes relative to TN90 expression also were analyzed to determine possible regulatory and/or biosynthetic genes that were highly expressed in N. alata but not in N. tabacum. Four targets were identified, which are listed in Table 3.

TABLE 3
Expression of genes highly expressed in N. alata

| | | | | | | | | | SEQ |
|---|---|---|---|---|---|---|---|---|---|
| | | | Root | Root | Root | | | | ID |
| Gene | TN90ª | N. alataª | (0 hr*) | (24 hr*) | (72 hr*) | Bud | Leaf | SEM | NO: |
| C31400 | 61 | 1469 | 355 | 735 | 584 | 182 | 437 | 276 | 43/44 |
| DC77221 | ND | 2754 | 1535 | 2313 | 1931 | 11 | 3 | 9 | 45/46 |
| C10055 | 150 | 1590 | 63 | 53 | 81 | 10 | 13 | 21 | 47/48 |
| C33728 | 1 | 8539 | 2328 | 2151 | 1677 | 2500 | 1298 | 2338 | 49/50 |

^{a}root tissues collected 72 hours after topping;
*hrs after topping

### Example 4—Sequence Alignments

The genes predicted to be involved with transport fall into five main categories: the nicotine uptake permease (Nup) family, the multidrug and toxic compound extrusion-type (MATE) family, the multiple drug resistance (MDR) family, and the pleiotropic drug resistance (PDR) family, as well as a group of unrelated genes. The nucleotide sequences and the predicted polypeptide sequences were compared with sequences deposited in public databases. The results of those comparisons are shown in Table 4 (Nup sequences), Table 5 (MDR sequences), Table 6 (MATE sequences), Table 7 (PDR sequences), Table 8 (other transporter sequences), and Table 9 (N. alata genes expressed at high levels).

TABLE 4
Nup sequences

| | Nuc | Prot | Protein | |
|---|---|---|---|---|
| Gene | ID % | ID % | Accession | Description |
| C32288 | 77 | 75 | ADP30798 | nicotine uptake permease 1 |
| | | | | [Nicotiana tabacum] |
| C40974 | 82 | 81 | ADP30798 | nicotine uptake permease 1 |
| | | | | [Nicotiana tabacum] |
| C42033 | 83 | 85 | ADP30799 | nicotine uptake permease 2 |
| | | | | [Nicotiana tabacum] |
| C29462 | 93 | 93 | ADP30798 | nicotine uptake permease 1 |
| | | | | [Nicotiana tabacum] |

TABLE 5
MDR sequences

| | Nuc | Prot | Protein | |
|---|---|---|---|---|
| Gene | ID % | ID % | Accession | Description |
| DC3222 | 86 | 87 | XP_004247427 | predicted: ABC transporter C |
| | | | | family member 4-like |
| | | | | [Solanum lycopersicum] |
| DC62783 | 87 | 89 | XP_004232253 | predicted: ABC transporter B |
| | | | | family member 15-like |
| | | | | [Solanum lycopersicum] |
| DC26451 | 90 | 91 | XP_004233862 | predicted: ABC transporter B |
| | | | | family member 9-like, partial |
| | | | | [Solanum lycopersicum] |
| C11099 | 84 | 85 | XP_004235187 | predicted: putative ABC transporter |
| | | | | B family member 8-like |
| | | | | [Solanum lycopersicum] |

TABLE 6
MATE sequences

| | Nuc | Prot | Protein | |
|---|---|---|---|---|
| Gene | ID % | ID % | Accession | Description |
| DC58421 | 69 | 71 | XP_004231608 | predicted: protein TRANSPARENT TESTA |
| | | | | 12-like [Solanum lycopersicum] |
| DC38072 | 79 | 78 | XP_004245689 | predicted: MATE efflux family protein |
| | | | | DTX1-like [Solanum lycopersicum] |
| C46276 | 78 | 84 | XP_004229626 | predicted: MATE efflux family protein |
| | | | | FRD3-like [Solanum lycopersicum] |
| C48594 | 76 | 87 | XP_004233485 | predicted: MATE efflux family protein 9-like |
| | | | | [Solanum lycopersicum] |
| C9954 | 52 | 87 | XP_004233485 | predicted: MATE efflux family protein 9-like |
| | | | | [Solanum lycopersicum] |
| C39106 | 63 | 67 | DAA50099 | TPA: putative MATE efflux family protein |
| | | | | [Zea mays] |
| C3055 | 81 | 88 | XP_004239125 | predicted: protein TRANSPARENT TESTA |
| | | | | 12-like [Solanum lycopersicum] |
| DC14012 | 86 | 85 | XP_004248692.1 | predicted: MATE efflux family protein 4, |
| | | | | chloroplastic-like [Solanum lycopersicum] |

TABLE 7
PDR sequences

| | Nuc | Prot | Protein | |
|---|---|---|---|---|
| Gene | ID % | ID % | Accession | Description |
| C53160 | 100 | 100 | BAD07484 | PDR-type ABC transporter 2 |
| | | | | [Nicotiana tabacum] |
| C22474 | 100 | 100 | AFN42938 | pleiotropic drug resistance |
| | | | | transporter 5b |
| | | | | [Nicotiana tabacum] |

TABLE 8
Other transporter sequences

| | Nuc | Prot | Protein | |
|---|---|---|---|---|
| Gene | ID % | ID % | Accession | Description |
| DC69629 | 89 | 92 | XP_004236321 | predicted: adenine/guanine |
| | | | | permease AZG2-like |
| | | | | [Solanum lycopersicum] |
| C43677 | 71 | 67 | XP_004235470.1 | predicted: bidirectional |
| | | | | sugar transporter SWEET 12-like |
| | | | | [Solanum lycopersicum] |
| C19125 | 98 | 91 | CCQ77797 | heavy metal ATPase |
| | | | | [Nicotiana tabacum] |

TABLE 9
Genes expressed at high levels in N. alata

| | Nuc | Prot | Protein | |
|---|---|---|---|---|
| Query | ID % | ID % | Accession | Description |
| C31400 | 88 | 91 | NP_001234027 | spe4 protein |
| | | | | [Solanum lycopersicum] |
| DC77221 | 82 | 81 | XP_004242860 | F-box/kelch-repeat |
| | | | | protein |
| | | | | [Solanum lycopersicum] |
| C10055 | 76 | 72 | XP_004239926 | proline transporter |
| | | | | 2-like |
| | | | | [Solanum lycopersicum] |
| C33728 | 100 | 100 | AAC49850.1 | DNA binding |
| | | | | protein ACBF |
| | | | | [Nicotiana tabacum] |

One of the genes that was highly expressed in N. alata relative to N. tabacum, C33728, appears to be an AC-rich binding factor (ACBF) regulatory protein. This polypeptide was shown to bind to AC-rich repeat regions in the promoter of heterologous xylem-related promoter from bean when transferred to tobacco, and was found to be expressed in all tissues but was most prevalent in the stem of the plant (Séguin et al., 1997, Plant Mol. Biol., 35(3):281-91). The polypeptide contains three distinct predicted RNA binding domains and a glutamine rich region that may be involved in gene activation. These predicted domains are shown with underlining in SEQ ID NO:50 below. The N-terminal domain is glutamine rich. This type of domain architecture is found in a number of splicing factors (Lorkovic and Barta, 2002, Nuc. Acids Res., 30:623-35).

(SEQ ID NO: 50)
MDGDAVSSSSNGDAATDDVWSAIHALQQHQQQQQKMQQSPTQIQSSSED
NKTIWIGDLQQWMDESYLHSCFSQAGEVISVKIIRNKQTGQSERYGFVE
FNTHAAAEKVLQSYNGTMMPNAEQPFRLNWAGFSTGEKRAETGSDFSIF
VGDLASDVTDTMLRDTFASRYPSLKGAKVVVDANTGHSKGYGFVRFGDE
SERSRAMTEMNGVYCSSRAMRIGVATPKKPSAQQQYSSQAVILSGGYAS
NGAATHGSQSDGDSSNTTIFVGGLDSDVTDEELRQSFNQFGEVVSVKIP
AGKGCGFVQFSDRSSAQEAIQKLSGAIIGKQAVRLSWGRSPANKQMRTD
SGSQWNGGYNGRQNYGGYGYGASQNQDSGMYATGAAYGASSNGYGNHQQ
PVS*

### Example 5—RNAi Line Development, Plasmid Construction and Transformation

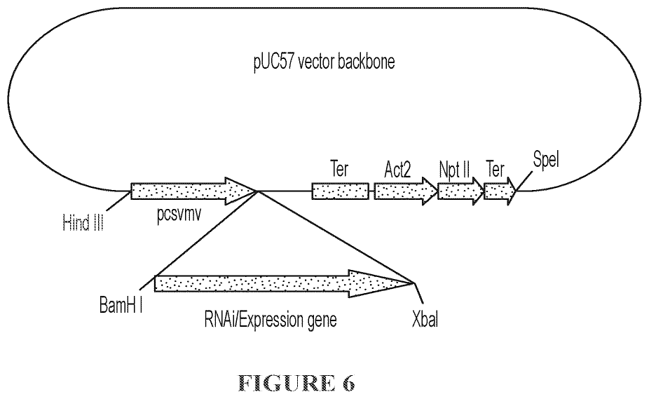

In order to evaluate the function of the candidate genes, two sets of transgenic plants were generated, one using the full length coding sequence and one using an RNAi sequence (see FIG. 6). For expression of the full length coding sequence or the RNAi sequence, an expression vector was used that has a CsVMV promoter and a NOS terminator, as well as a cassette having a kanamycin selection marker (NPT II) under direction of the actin2 promoter and having the NOS terminator. The nucleic acid constructs carrying the transgenes of interest were introduced into tobacco leaf disc using DNA bombardment or a biolistic approach. See, for example, Sanford et al., 1993, Methods Enzymol., 217:483-510; and Okuzaki and Tabei, 2012, Plant Biotechnology, 29:307-310.

Briefly, the plasmid DNA containing the transformation cassette was coated on 1 μm gold particles (DNA/gold) as follows. The 1 μm gold particles were baked at 180° C. for 12 hours, and a stock solution (40 mg/ml) was prepared. To make a mixture for 10 shots, 100 μl of the stock solution was mixed with 40 μl of expression vector DNA (1 μg/μl), 100 μl of 2.5 M CaCl₂, and 40 μl of 0.1 M spermidine in a 1.5-ml tube. The mixture was centrifuged for 30 s at 13,000×g, and the pellet was washed with 500 μl 100% ethanol. The DNA/gold mixture was suspended in 100 μl of water, and 10 μl was applied onto a macrocarrier, dried, and then bombarded. Two shots were bombarded per plate using a 1,100 psi rupture disc under partial vacuum (711 mmHg) in a PDS-1000/He system (Bio-Rad Laboratories, Hercules, CA, USA).

Narrow Leaf Madole (NLM) and Tennessee 90 (TN90) tobacco leaf discs were used for transformation with the RNAi constructs, and Nicotiana alata tobacco leaf discs were used for transformation with the full length candidate gene constructs. Whole tobacco leaf (about 45×30 mm in length) was placed on the MS medium overnight, and the leaf disc was bombarded with the construct on the second day. Leaves were then cut into small pieces (about 5×5 mm) and replaced on the TOM medium (MS medium with 20 g sucrose/L; 1 mg/L IAA and 2.5 mg/L BAP) to grow at 27° C. for 3-5 days, then transferred to TOM medium to grow, which contains 300 mg/l Kanamycin (TOM-Kan). Tissues were transferred to new TOM-Kan plates every 2-3 weeks for 4-6 weeks (27° C., 16 h light). Kanamycin-resistant primary shoots were regenerated at 4-6 weeks after bombardment. Shoots were transferred to MS-Kanamycin plates to grow root.

The leaves and/or roots from T1 plants (and subsequent generations) are evaluated to determine the amount of one or more alkaloids and/or one or more TSNAs.

### Example 6—Sequences of RNAi Molecules and Expression Construct

RNAi molecule sequences are shown below. The double-underlined portion is the loop, which is from the tobacco QS gene sequence.

| pALCS-TDNA-R1 RNAi sequence | |
|---|---|
| (22474; SEQ ID NO: 51) | |
| GGATCCAAAG AGCAGGCCAG CGATATGGAA GCTGATCAAG AAGAAAGCAC GGGAAGCCCA | 60 |
| AGACTTAAAA TCAGCCAGTC GAAGAGAGAT GATCTCCCTC GATCCTTATC TGCAGCAGAT | 120 |
| GGAAATAAGA CAAGAGAAAT GGAAATCCGA CGAATGAGCA GTCATATCCA TTCTAGTGGC | 180 |
| CTCTACAGAA ATGAGGATGC AAATCTTGAG GCTGCAAATG GTGTCGCAGG TTCTTTACTT | 240 |
| GAACATTTTA GGAATTTAGG AAATGCTTGT TCGTCATTTG TTTTGTGTCC TAGCCTATTG | 300 |
| TTTATTGTTT GTTTTTATCT TCACTTTAGT GAGGATACAT ATTCTGAGCA CACTCTGAAA | 360 |
| ATATAGCTCA TTTATGTTTA TAGGGAAAGG AGAAAAGAGA GAGTCACATC ATGGCAACTG | 420 |
| CGACACCATT TGCAGCCTCA AGATTTGCAT CCTCATTTCT GTAGAGGCCA CTAGAATGGA | 480 |
| TATGACTGCT CATTCGTCGG ATTTCCATTT CTCTTGTCTT ATTTCCATCT GCTGCAGATA | 540 |
| AGGATCGAGG GAGATCATCT CTCTTCGACT GGCTGATTTT AAGTCTTGGG CTTCCCGTGC | 600 |
| TTTCTTCTTG ATCAGCTTCC ATATCGCTGG CCTGCTCTTT TCTAGA | 646 |
| pALCS-TDNA-R4 RNAi sequence | |
| (43677; SEQ ID NO: 52) | |
| GGATCCGTTT TGGTCCTTTG CATAAAATTT GGGTAAGGAA AAGAATCAAT CCAAAGCCAC | 60 |
| CGAAATTCAA CAGTAGGACA AGTCTCAGTG TTTGCATCTG CAAAACCATT CCAAATTTCA | 120 |
| CAATCCACGA AACTGTGTAA CAATAACTAA AACATAACGA AATAAATACT AGGAGTATAA | 180 |
| TCTATAGGCA CAAAATTGAA GTTGTGCATG TTCTTTACTT GAACATTTTA GGAATTTAGG | 240 |
| AAATGCTTGT TCGTCATTTG TTTTGTGTCC TAGCCTATTG TTTATTGTTT GTTTTTATCT | 300 |
| TCACTTTAGT GAGGATACAT ATTCTGAGCA CACTCTGAAA ATATAGCTCA TTTATGTTTA | 360 |
| TAGGGAAAGG AGAAAAGAGA GAGTCACATC ATGGATGCAC AACTTCAATT TTGTGCCTAT | 420 |
| AGATTATACT CCTAGTATTT ATTTCGTTAT GTTTTAGTTA TTGTTACACA GTTTCGTGGA | 480 |
| TTGTGAAATT TGGAATGGTT TTGCAGATGC AAACACTGAG ACTTGTCCTA CTGTTGAATT | 540 |
| TCGGTGGCTT TGGATTGATT CTTTTCCTTA CCCAAATTTT ATGCAAAGGA CCAAAACGTC | 600 |
| TAGA | 604 |
| pALCS-TDNA-R2 RNAi sequence | |
| (11099; SEQ ID NO: 53) | |
| GGATCCAAAG ATTTCAAGGA CCTTATTTAT GCTCCCAAAC GAGGTCACAA TCCTATGATT | 60 |
| ATAAACAGCC TCCACAGCAG TTTGAGTGCT TTGATATTGT GCCTTGACGA ACTTAGCTGT | 120 |
| GATGGTGGAT AGCAAGACTT TTCGCGTGTA AAAGCATAGA ATTGTGAGAG GTTGGACAGC | 180 |
| AATCATAACT AGTGCAAGCT TCCAAGCGTT CTTTACTTGA ACATTTTAGG AATTTAGGAA | 240 |
| ATGCTTGTTC GTCATTTGTT TTGTGTCCTA GCCTATTGTT TATTGTTTGT TTTTATCTTC | 300 |
| ACTTTAGTGA GGATACATAT TCTGAGCACA CTCTGAAAAT ATAGCTCATT TATGTTTATA | 360 |
| GGGAAAGGAG AAAAGAGAGA GTCACATCAT GGCAAGCTTG GAAGCTTGCA CTAGTTATGA | 420 |
| TTGCTGTCCA ACCTCTCACA ATTCTATGCT TTTACACGCG AAAAGTCTTG CTATCCACCA | 480 |
| TCACAGCTAA GTTCGTCAAG GCACAATATC AAAGCACTCA AACTGCTGTG GAGGCTGTTT | 540 |
| ATAATCATAG GATTGTGACC TCGTTTGGGA GCATAAATAA GGTCCTTGAA ATCTTTGTCT | 600 |
| AGA | 603 |
| pALCS-TDNA-R7 RNAi sequence | |
| (40974; SEQ ID NO: 54) | |
| GGATCCGTGT CAACCTCTTC ACTTCTTCTT GCTGCTCAAC TTGCCTTCAC GGCAATAGGT | 60 |
| GCTTTCTTCA TAGTGAAGCT GAAATTCACA CCCTACTCTA TCAATGCAGT GGTTCTGTTG | 120 |
| ACAGTTGGTG CTGTTTTATT AGGTATTCGA TCAAATGGTG ATCGGCCAGA GGGTGTGACA | 180 |
| AGTAGAGCTT ATATTTACTC TTTGTTCTTT ACTTGAACAT TTTAGGAATT TAGGAAATGC | 240 |
| TTGTTCGTCA TTTGTTTTGT GTCCTAGCCT ATTGTTTATT GTTTGTTTTT ATCTTCACTT | 300 |
| TAGTGAGGAT ACATATTCTG AGCACACTCT GAAAATATAG CTCATTTATG TTTATAGGGA | 360 |
| AAGGAGAAAA GAGAGAGTCA CATCATGGCA AAGAAAGATA AACCTTTAGT ACTAGTGAGG | 420 |
| CTTGAACGTC TGTGACATTT AAAGTCCTAA GTTAGTTTCT ATTGTAATTG AATATAAGCT | 480 |
| CTACTTGTCA CACCCTCTGG CCGATCACCA TTTGATCGAA TACCTAATAA AACAGCACCA | 540 |
| ACTGTCAACA GAACCACTGC ATTGATAGAG TAGGGTGTGA ATTTCAGCTT CACTATGAAG | 600 |
| AAAGCACCTA TTGCCGTGAA GGCAAGTTGA GCAGCAAGAA GAAGTGAAGA GGTTGACACT | 660 |
| CTAGA | 665 |
| pALCS-TDNA-R3 RNAi sequence | |
| (46276; SEQ ID NO: 55) | |
| GGATCCACAC CATCTAAAAC AAACGCCAAT GAGTTGATTG GTTGTGTACC AGCGACAAAC | 60 |
| TGGCGAAGGA GCACAAGGTA AAAAGAACAG TTAAAGATAG TGAAACAAAA GAAGAACAAA | 120 |
| TTGAAACAAA CATATAGTAC TACTATTTAT TGAATGTATA CCGGGATGGC AATGGTTATG | 180 |
| AGACGGATAA CATTTTTGTC CTTTGAGTTC TTTACTTGAA CATTTTAGGA ATTTAGGAAA | 240 |
| TGCTTGTTCG TCATTTGTTT TGTGTCCTAG CCTATTGTTT ATTGTTTGTT TTTATCTTCA | 300 |
| CTTTAGTGAG GATACATATT CTGAGCACAC TCTGAAAATA TAGCTCATTT ATGTTTATAG | 360 |
| GGAAAGGAGA AAAGAGAGAG TCACATCATG GCAATCAAAG GACAAAAATG TTATCCGTCT | 420 |
| CATAACCATT GCCATCCCGG TATACATTCA ATAAATAGTA GTACTATATG TTTGTTTCAA | 480 |
| TTTGTTCTTC TTTTGTTTCA CTATCTTTAA CTGTTCTTTT TACCTTGTGC TCCTTCGCCA | 540 |
| GTTTGTCGCT GGTACACAAC CAATCAACTC ATTGGCGTTT GTTTTAGATG GTGTTCTAGA | 600 |
| pALCS-TDNA-R5 RNAi sequence | |
| (39106; SEQ ID NO: 56) | |
| GGATCCATGG GCTGATGTTC ATGGTTTCAA TGGGGTTCAA TGCTGCTGCT AGTGTAAGGG | 60 |
| TGAGCAATGA GTTAGGAGCA CCACACCCAA AGTCAGCAGC ATTCTTAGTG TTTGTGGTGA | 120 |
| CATTCATTTC ATTTCTCATA GCTGTGGTGG AAGCCATAAT TATGCTGTGT TTGCGCAATG | 180 |
| TGATCAGCTA TGCATTCACT AAGGGTTACT CTTTGTTCTT TACTTGAACA TTTTAGGAAT | 240 |
| TTAGGAAATG CTTGTTCGTC ATTTGTTTTG TGTCCTAGCC TATTGTTTAT TGTTTGTTTT | 300 |
| TATCTTCACT TTAGTGAGGA TACATATTCT GAGCACACTC TGAAAATATA GCTCATTTAT | 360 |
| GTTTATAGGG AAAGGAGAAA AGAGAGAGTC ACATCATGGC AAAGAAAGAT AAACCTTTAG | 420 |
| TACTAGTGAG GCTTGAACGT CTGTGACATT TAAAGTCCTA AGTTAGTTTC TATTGTAATT | 480 |
| GACCCTTAGT GAATGCATAG CTGATCACAT TGCGCAAACA CAGCATAATT ATGGCTTCCA | 540 |
| CCACAGCTAT GAGAAATGAA ATGAATGTCA CCACAAACAC TAAGAATGCT GCTGACTTTG | 600 |
| GGTGTGGTGC TCCTAACTCA TTGCTCACCC TTACACTAGC AGCAGCATTG AACCCCATTG | 660 |
| AAACCATGAA CATCAGCCCA TTCTAGA | 687 |

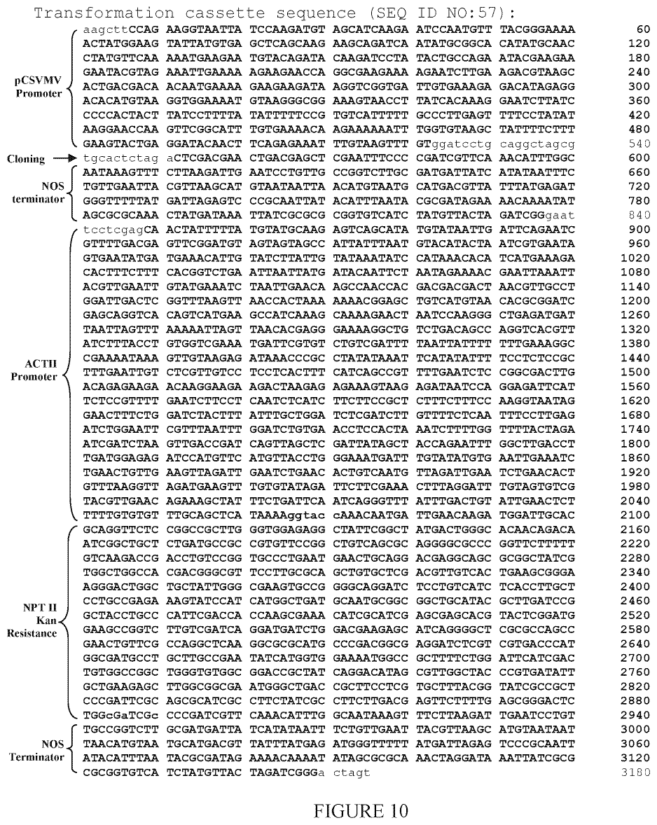

The sequence of the expression cassette is shown in FIG. 10, with the relevant portions indicated in the left margin. See also FIG. 6.

### Example 7—Random Mutagenesis and Characterization of Mutants

For EMS mutation, one gram (approximately 10,000 seeds) of Tennessee 90 tobacco (TN90) converter seed was washed in 0.1% Tween® for fifteen minutes and then soaked in 30 ml of ddH₂O for two hours. One hundred fifty (150) μl of 0.5% EMS (Sigma, Catalog No. M-0880) was then mixed into the seed/ddH₂O solution and incubated for 8-12 hours (rotating at 30 rpm) under a hood at room temperature (RT; approximately 20° C.). The liquid then was removed from the seeds and the liquid was mixed into 1 M NaOH overnight for decontamination and disposal. The seeds were then washed twice with 100 ml ddH₂O for 2-4 hours. The washed seeds were then suspended in 0.1% agar:water solution.

The EMS-treated seeds in the agar solution were evenly spread onto water-soaked Carolina's Choice Tobacco Mix3 (Carolina Soil Company, Kinston, NC) in flats at ˜2000 seeds/flat. The flats were then covered with plastic wrap and placed in a growth chamber. Once the seedlings emerged from the soil, the plastic wrap was punctured to allow humidity to decline gradually. The plastic wrap was completely removed after two weeks. Flats were moved to a greenhouse and fertilized with NPK fertilizer. The seedlings were plugged into a float tray and grown until transplanting size. The plants were transplanted into a field. During growth, the plants were self-pollinated to form M₁ seeds. At the mature stage, five capsules were harvested from each plant and individual designations were given to the set of seeds from each plant. This formed the M₁ population.

A composite of M₁ seed from each M₀ plant was grown, and leaves from M₁ plants were collected and DNA extracted. Target genes were amplified and sequenced for mutation identification.

### Example 8—Targeted Mutagenesis Using TALENs

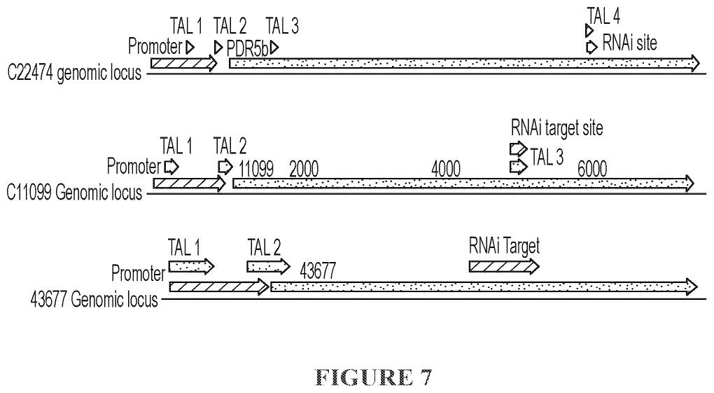

Gene specific TALEN recognition sequences were found within either the specific gene targets or within the promoter sequence that allow for targeted deletions or promoter insertions in order to reduce expression of the gene or change the tissue-specific expression. The sequences of the regions of interest are shown below (SEQ ID NOs: 61-69). The specific target sequences for gene C22474 (Pdr5b) are underlined in the corresponding sequence of the region of interest. The locations of all of the TALEN regions of interest are shown schematically in FIG. 7. The yellow bar denotes the primary transcript including introns. The green denotes the upstream region which has been marked as the promoter region. These TALEN sites are specific for the single gene target based on known genomic sequence information. TALEN regions 1 and 2 for each gene are used to disrupt the promoter or 5′ end of the coding region, while other regions are used to disrupt only the coding sequence.

C22474-TAL1
(SEQ ID NO: 61)
AATTCAAACCTGTCAAAACCATAAAAAGATATTGGACAAATGCTTTTAA
TATAATTGCCTTAGATTAATCTATATATATATATATATATATATAGGTA
AATACTTACTTGTATCAGACATTTATCTTTATAAATATGTTATTCACTA
AATCATAGTTAATTAATATATATTTTTACCTTAAGGGGCCGTTTGGTTG
GGAAA
C22474-TAL2
(SEQ ID NO: 62)
TATCGCACTACTATTGAACCTATCGCCTTTTGAGTTTTGATATATAAAT
AGCGACGAACGTTTCTTAGATAATGGACTCATAACCTCCCTCTTCACAA
CTAGAAGAGCGTGAGACCTTTTCAATTAGAATTCGTAGGAAAAAATCAA
ACACAAATTCACAAAACAAAAATTTATTAAGATTTCAGCGACCAAGCCC
GTGAG
C22474-TAL3
(SEQ ID NO: 63)
TCGTTTGAGAAAAACGGTCCTTAAATCGGTCATGGAAAGTGAGAATAAT
CAGGGCAATAAAAAAGTTGTTCATAAGGAAGTTGATGTTCGGAATCTGG
GATTGAATGAGCGACAAGAGTTCATTGATCGATTTTTCAGGGTTGCTGA
GGAAGATAATGAAAAGTTTCTGAGAAAGTTCAGAAATCGAATTGACAAG
TAAGTTTCCAGTATTACT
C22474-TAL4
(SEQ ID NO: 64)
ACAAGCCACAAGCTACACTATCCAAAGAGCAGGCCAGCGATATGGAAGC
TGAGCAAGAAGAAAGCACGGGAACCCCTAGACTTCGAATCAGCCAGTCG
AAGAGAGATGATCTCCCTCGATCCTTATCTGCAGCAGATGGGAACAAGA
CAAGTATGATCTTTAGCCCATCAATAACAGAATCTGCTTGGGGAATATA
AGTAATGCTTACAGT
C11099-TAL1
(SEQ ID NO: 65)
ATACGATAAGTCCTCTTAAAATTACCATACTTATAAAGTCATAAAAGTA
GAAAGAAAAAGGACCTCTTTGAAAATTTTTATATAAAAGGGGCTGAAAA
TATGCGATAATGTCAAGTAGCAGTTTGGCTTCATATATTGGTCCATGTT
ATCGGAGTTGGTATTTATGTTAAATATTAAGTACTTTTTTATCATATCT
ATCA
C11099-TAL2
(SEQ ID NO: 66)
ACTCAATTTCTGCCACTTTATTATAAATAGTAAGTTAGTATTCCATTCT
TGGTCAGAAAGGAGTATGGGAAATCAAGGTCTATTTTCTTAGTTACAGA
CCTAACAATTTCCATTGTCACCTTTTTTCAGCTGTTGGCGTGTAGAAAC
GGACCTTTGAGCATTGTTGATGCGTTTGACTTGTTTTAGAAAAGAAAAA
AGAATG
C11099-TAL3
(SEQ ID NO: 67)
GGCTAGTTGTGGCTTGGAAGCTTGCACTAGTTATGATTGCTGTCCAACC
TCTCACAATTCTATGCTTTTACACGCGAAAAGTCTTGCTATCCACCATC
ACAGCTAAGTTCGTCAAGGCACAATATCAAAGCACTCAAACTGCTGTGG
AGGCTGTTTATAATCATAGGATTGTGACCTCGTTTGGGAGCATAAATAA
GGTCCTTGAAATCTTTGATGAGGCACAGGATGAGTCAA
C43677-TAL1
(SEQ ID NO: 68)
TTTTTAACCACCTAGTGGATGCTAATATGGTGTCAGCATTAGAAGAAAC
TAATTCATGATTTAAGTTTTATAGGTTCAATTTTTAGATTTTTAATATT
AAATATATTATATTTTAAAGTTATGAGTTAATATTTGTTGAAGTATTTG
TTAAGTATAATTATAATAAATTTTAACACTAATATTTATATTTATGCTC
TGCGTCAACAG
C43677-TAL2
(SEQ ID NO: 69)
TTTTTAACCACCTAGTGGATGCTAATATGGTGTCAGCATTAGAAGAAAC
TAATTCATGATTTAAGTTTTATAGGTTCAATTTTTAGATTTTTAATATT
AAATATATTATATTTTAAAGTTATGAGTTAATATTTGTTGAAGTATTTG
TTAAGTATAATTATAATAAATTTTAACACTAATATTTATATTTATGCTC
TGCGTCAACAG

The target sequences of the genes of interest are sent to Life Technology (Carlsbad, CA) to determine possible binding site sequences of the transcription activator like (TAL) effector proteins. The TALs are synthesized and cloned into the inventors' plant expression vector, pALCS1, by Life Technology to serve as entry vectors. Depending on the purpose, five different protocols can be used to generate mutagenic tobacco lines: 1) one or more entry vectors (pALCS1 containing the target TALs) are directly transformed into tobacco protoplasts to generate random sequence deletion or insertion mutagenic tobacco lines; 2) a donor sequence (e.g., a reporter gene, e.g., the GUS gene) flanked on the left and right side with sequences that are homologous with the target insertion sequence is co-transformed into tobacco protoplasts with one or more entry vectors (pALCS1 containing the target TALs) to generated mutagenic tobacco lines containing a reporter gene; 3) a donor sequence containing target TALs that have a point mutation is co-transformed into tobacco protoplasts with one or more entry vectors (pALCS1 containing the target TALs) to generated mutagenic tobacco lines having a point mutation; 4) a donor sequence containing a tissue specific promoter sequence to generate mutant tobacco lines that express the endogenous gene in a tissue specific manner; and 5) a donor sequence containing a combination of the aforementioned donor sequences with a reporter gene construct to facilitate mutant tobacco screening.

Tobacco protoplasts are isolated from TN90 tobacco leaves growing in Magenta boxes in a growth chamber. Well-expanded leaves (5 cm) from 3-4-week-old plants are cut into 0.5 to 1-mm leaf strips from the middle part of a leaf. Leaf strips are transferred into the prepared enzyme solution (1% cellulase R10, 0.25% macerozyme R10, 0.4 M mannitol, 20 mM KCl, 20 mM MES (pH 5.7), 10 mM CaCl₂, 0.1% BSA) by dipping both sides of the strips. Leaf strips are vacuum infiltrated for 30 min in the dark using a desiccator with continuing digestion in the dark for 4 hour to overnight at room temperature without shaking. Protoplasts are filtered in 100 μm nylon filter and purified with 3 ml Lymphoprep. Protoplasts are centrifuged and washed with W5n solution (154 mM NaCl, 125 mM CaCl₂, 5 mM KCl, 2 mM MES, 991 mg/l glucose pH 5.7) and suspended in W5n solution at the concentration of 5×10⁵/ml. Protoplasts are kept on ice for 30 minutes to settle at the bottom of the tube by gravity. W5n solution was moved and protoplasts were re-suspended in P2 solution at room temperature. 50 μl DNA (10-20 μg of plasmid), 500 μl protoplasts (2×10⁵ protoplasts) and 550 μl of PEG solution (40%, v/v 10 ml 4 g PEG4000, 0.2 M mannitol, 0.1 M CaCl₂) are mixed gently in a 15 ml microfuge tube, and the mixture incubated at room temperature for 5 minutes.

Protoplasts are pelleted and re-suspended with 1 ml 2×8EN1 (8EN1: MS salt without NH₄NO₃, MS vitamin, 0.2% myo-Inositol, 4 mM MES, 1 mg/l NAA, 1 mg/l IAA, 0.5 M mannitol, 0.5 mg/l BAP, 1.5% sucrose). Transformed protoplasts are jellified with equal amount of low-meting agarose (LMA), and 0.2 ml of protoplast-LAM is dropped to form a bead. 10 ml 8EN1 is added to the bead, and in 7 days, 5 ml 8EN1 is taken out and 5 ml 8EN2 (8EN1 with 0.25 M mannitol) is added; after another 7 days (14 day), 10 ml 8EN2 is taken out and 10 ml 8EN2 is added; in another 7 days (21 day), 5 ml 8EN2 is taken out and 5 ml 8EN3 (8EN1 with 3% sucrose and without mannitol) is added; after another 7 days (28 day), 10 ml 8EN3 is taken out and 10 ml 8EN3 is added. Protoplasts are kept for two weeks until micro-callus growth. Callus is transferred to NCM solid media until it reaches about 5 mm (usually about two weeks). Callus was transferred to TOM-Kan solid media to grow shoots, and transformed tobacco plants were regenerated using the methods described herein.

TABLE 10
TAL effector binding site sequences

| | | SEQ |
|---|---|---|
| TALEN | Target sequence | ID NO: |
| TALEN site 1 | TATGTTATTCACTAAATCATAGTTAATTAAT | 58 |
| | ATATATTTTTACCTTA | |
| TALEN site 2 | TTCACAACTAGAAGAGCGTGAGACCTTTTCA | 59 |
| | ATTAGAATTCGTAGGA | |
| TALEN site 3 | TCCTTAAATCGGTCATGGAAAGTGAGAATAA | 60 |
| | TCAGGGCAATAAAAAA | |

underlining = TAL binding sites; non-underlining region
between TAL binding sites = position where DNA cleavage
is designed to occur

### Example 9—Screening Plants for Modulation of Alkaloid Content

Transgenic and mutant tobacco plants identified in Examples 5, 6, 7 or 8 are grown in a greenhouse under field-like conditions in Carolina's Choice Tobacco Mix (Carolina Soil Co., Kinston, NC) in 10 inch pots. At flowering stage, the plants are topped and tissue samples are collected from expanded leaves and roots 2 weeks later.

The tissue is ground in a mortar and pestle. Alkaloid content is determined by gas chromatography coupled to mass spectroscopy using certified protocols from Arista Laboratories. The amount of nicotine, nornicotine, anabasine, and anatabine, as well as the total alkaloid content, are determined.

### Example 10—Nicotine Feeding Assay

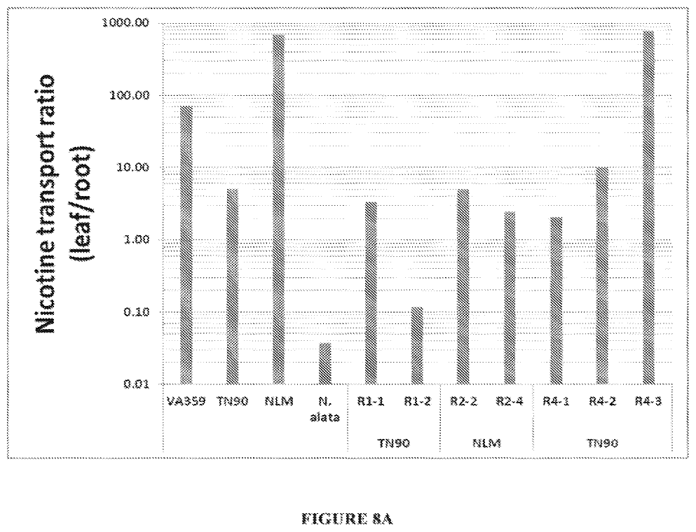

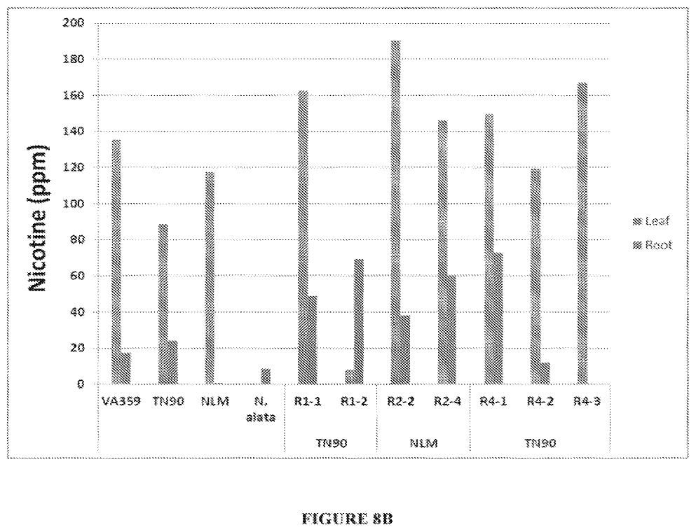

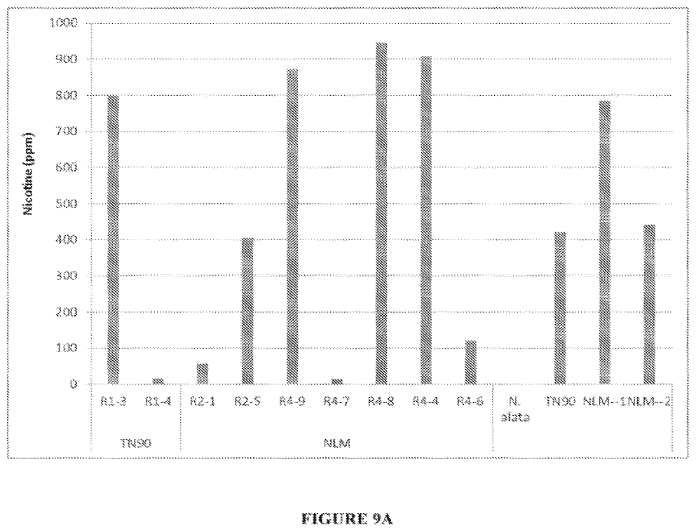

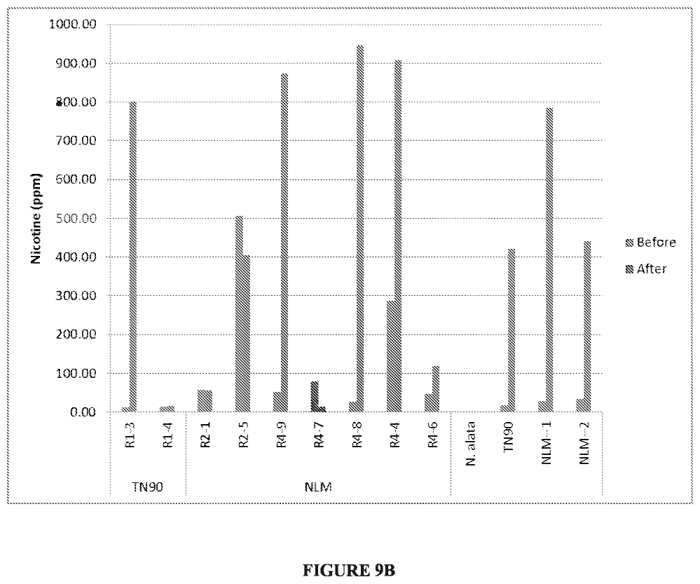

The transport of nicotine in young transgenic plants can be tested by feeding nicotine along with the fertilized water to boost the amount of nicotine and determine the phenotype much earlier than waiting for the endogenous alkaloids to be measurable. Feeding assays were conducted by two separate methods. In the first method, seedlings were transferred to a Styrofoam float tray. These plants were allowed to grow on 100 ppm fertilized water (Pete's Professional) until roots began to emerge from the bottom of the tray. The trays were then floated on fertilized nicotine solution (1 mM nicotine, 100 ppm fertilizer) for three days. Root and leaf tissue was harvested and alkaloids were extracted and analyzed as described above. Nicotine content of the roots and leaves were calculated as well as the ratio of leaf to root nicotine levels (FIG. 8). In the second method, plants were transferred to 4 inch pots. After the roots had grown enough to hold the soil together, these were transferred to 4 inch pots with the bottoms removed. After 2 weeks of growth, plants were treated twice a day with fertilized nicotine solution for 2 days. Leaves were harvested before and after nicotine feeding and alkaloids were extracted and analyzed as described above. The nicotine content of the leaves before and after feeding is shown in FIG. 9.

Reduced nicotine levels were found in two plants expressing RNAi constructs targeting the PDR family gene, C22474 (pALCS-TDNA-R1): one plant expressing the RNAi construct targeting the MDR family gene 11099 (pALCS-TDNA-R2) showed reduced nicotine levels after feeding; and one plant expressing the RNAi construct targeting the unclassified gene C43677 (pALCS-TDNA-R4) also showed reduced nicotine levels in the leaf. The locations of the RNAi targeted sites are shown in FIG. 7.

### Example 11—Comparison of N. tabacum with N. otophora

N. tabacum originated from the hybridization of two distinct lineages of Nicotiana. N. alata represents a member of the N. sylvestris lineage that does not transport alkaloids (see Example 3 above). N. otophora is a non-transporting member of the other lineage (N. tomentosiformis). RNA sequencing was conducted to determine transport associated genes that are missing from N. otophora but are present at high levels in N. tabacum.

Briefly, root samples from N. otophora and N. tabacum TN90 were collected 10 days after topping. RNA was extracted and sequencing was done by Ambry Genetics (Aliso Viejo, CA). RNA sequencing generated 125 million sequence reads. Denovo contig sequences were generated for N. otophora and previously obtained genomic sequence from TN90 was used for comparison. Sequence reads were mapped to the denovo N. otophora contig sequences, and the remaining unmapped reads were then mapped to the TN90 genome to generate a list of genes that would be predicted to be missing from N. otophora. Genes related to secondary metabolite transport were chosen based on gene ontology terms. The expression levels of these genes at various time points are shown in Table 11.

TABLE 11
Gene expression in TN90

| | | | | | | | | | SEQ ID |
|---|---|---|---|---|---|---|---|---|---|
| | Gene | Root | Root | Root | | | | N. | NO |
| | Desig. | (0 hr*) | (24 hr*) | (72 hr*) | BUD | SEM | Leaf | otophora | (nt/prot) |
| MDR | g192339 | 173 | 316 | 209 | 101 | 69 | 121 | ND | 70/71 |
| | g192334 | 312 | 568 | 410 | 156 | 106 | 261 | ND | 72/73 |
| | g190446 | 852 | 1454 | 1391 | 464 | 416 | 386 | ND | 74/75 |
| | g124216 | 6564 | 4838 | 4545 | 10359 | 11259 | 5844 | ND | 76/77 |
| | g132727 | 114 | 88 | 48 | 161 | 137 | 105 | ND | 78/79 |
| | g84371 | 461 | 443 | 426 | 591 | 524 | 431 | ND | 80/81 |
| NUP | g144767 | 2313 | 2679 | 2202 | 370 | 190 | 404 | ND | 82/83 |
| Other | g195231 | 976 | 2472 | 1555 | 499 | 524 | 3382 | ND | 84/85 |
| MATE | g105138 | 953 | 1692 | 1356 | 524 | 485 | 1613 | ND | 86/87 |
| | g127664 | 499 | 605 | 451 | 160 | 192 | 134 | ND | 88/89 |
| | g173763 | 893 | 1521 | 1381 | 431 | 300 | 804 | ND | 90/91 |

*hrs after topping;
ND = not detected

The nucleotide sequences and the predicted polypeptide sequences were compared with sequences deposited in public databases. The results of those comparisons are shown in Table 12.

TABLE 12
Sequence Alignments

| | Nuc | Prot | Protein | |
|---|---|---|---|---|
| Gene | ID % | ID % | Accession | Description |
| g144767 | 100 | 100 | XP_009767545.1 | amino acid permease 3-like |
| | | | | [Nicotiana sylvesteris] |
| g192339 | 99 | 95 | XP_009759241 | ABC transporter A family member 2 |
| | | | | [Nicotiana sylvesteris] |
| g192334 | 99 | 99 | XP_009759239 | ABC transporter A family member 7-like isoform |
| | | | | X1 [Nicotiana sylvesteris] |
| g124216 | 100 | 100 | XP_009788997 | ABC transporter F family member 1 |
| | | | | [Nicotiana sylvesteris] |
| g132727 | 99 | 99 | XP_009760683 | ABC transporter G family member 11-like |
| | | | | [Nicotiana sylvesteris] |
| g84371 | 86 | 69 | XP_006363174 | ABC transporter G family member 31-like |
| | | | | [Solanum tuberosum] |
| g190446 | 99 | 92 | XP_009768405 | putative ABC transporter C family member 15 |
| | | | | isoform X1 [Nicotiana sylvesteris] |
| g105138 | 95 | 88 | XP_009760570.1 | protein transperant testa 12-like |
| | | | | [Nicotiana sylvesteris] |
| g173763 | 99 | 94 | XP_009760619.1 | protein transperant testa 12-like |
| | | | | [Nicotiana sylvesteris] |
| g127664 | 99 | 99 | XP_009768477.1 | protein transperant testa 12-like |
| | | | | [Nicotiana sylvesteris] |
| g195231 | 100 | 100 | XP_009800568.1 | polyol transporter 5-like |
| | | | | [Nicotiana sylvesteris] |

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

What is claimed is:

1. A tobacco hybrid, variety, line, or cultivar comprising plants having a mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90.

2. The tobacco hybrid, variety, line, or cultivar of claim 1, wherein leaf from the plants exhibit a reduced amount of at least one alkaloid relative to leaf from a plant lacking the mutation.

3. The tobacco hybrid, variety, line, or cultivar of claim 2, wherein cured leaf from the plants exhibit a reduced amount of at least one tobacco specific nitrosamine (TSNA) relative to cured leaf from a plant lacking the mutation.

4. Seed produced by the tobacco hybrid, variety, line, or cultivar of claim 1, the seed comprising the mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90.

5. A method of making a tobacco plant, comprising the steps of:
inducing mutagenesis in Nicotiana tabacum cells to produce mutagenized cells;
obtaining one or more plants from the mutagenized cells; and
identifying at least one of the plants that comprises a mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90.

6. The method of claim 5, further comprising identifying at least one of the plants that comprises leaf exhibiting a reduced amount of at least one alkaloid relative to leaf from a plant lacking the mutation.

7. The method of claim 6, further comprising identifying at least one of the plants that comprises leaf that, when cured, exhibit a reduced amount of at least one TSNA relative to cured leaf from a plant lacking the mutation.

8. The method of claim 5, wherein mutagenesis is induced using a chemical mutagen or ionizing radiation.

9. The method of claim 8, wherein the chemical mutagen is selected from the group consisting of nitrous acid, sodium azide, acridine orange, ethidium bromide, and ethyl methane sulfonate (EMS).

10. The method of claim 8, wherein the ionizing radiation is selected from the group consisting of x-rays, gamma rays, fast neutron irradiation, and UV irradiation.

11. The method of claim 5, wherein mutagenesis is induced using TALEN.

12. The method of claim 5, wherein mutagenesis is induced using zinc-finger technology.

13. A method for producing a tobacco plant, said method comprising the steps of:
crossing at least one plant of a first tobacco line with at least one plant of a second tobacco line, the plant of the first tobacco line having a mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90; and
selecting for progeny tobacco plants that have the mutation.

14. The method of claim 13, further comprising selecting for progeny tobacco plants that comprise leaf exhibiting a reduced amount of at least one alkaloid relative to leaf from a plant lacking the mutation.

15. The method of claim 14, further comprising selecting for progeny tobacco plants that comprise leaf that, when cured, exhibit a reduced amount of at least one TSNA relative to cured leaf from a plant lacking the mutation.

16. A tobacco product comprising cured leaf from a tobacco plant having a mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90.

17. The tobacco product of claim 16, wherein the leaf exhibits a reduced amount of at least one alkaloid relative to leaf from a plant lacking the mutation.

18. The tobacco product of claim 17, wherein the cured leaf exhibits a reduced amount of at least one TSNA relative to cured leaf from a plant lacking the mutation.

19. A method of producing a tobacco product, the method comprising:
providing cured leaf from a tobacco plant having a mutation in one or more endogenous nucleic acids having a sequence selected from the group consisting of SEQ ID NOs: 1, 3, 5, 7, 9, 11, 13, 15, 17, 19, 21, 23, 25, 27, 29, 31, 33, 35, 37, 39, 41, 43, 45, 47, 49, 70, 72, 74, 76, 78, 80, 82, 84, 86, 88, and 90; and
manufacturing a tobacco product using the cured leaves.

20. The method of claim 19, wherein the cured leaf exhibits a reduced amount of at least one alkaloid relative to cured leaf from a plant lacking the mutation.

21. The method of claim 19, wherein the cured leaf exhibits a reduced amount of at least one TSNA relative to cured leaf from a plant lacking the mutation.

22. The method of claim 19, wherein the mutation is selected from the group consisting of a point mutation, an insertion, a deletion, and a substitution.

23. A method of screening plants, comprising:
providing plant material from the plant of claim 1; and
determining the amount of one or more alkaloids in plant tissue.

24. The method of claim 23, wherein the plant tissue is leaf.

25. The method of claim 23, wherein the plant tissue is root.
